(19) United States
(12) Patent Application Publication
(10) Pub. No.: US 2009/0175922 A1
(43) Pub. Date: Jul. 9, 2009

(54) THREE DIMENSIONAL PURIFIED COLLAGEN MATRICES

(76) Inventor: Sherry L. Voytik-Harbin, Zionsville, IN (US)

Correspondence Address: BARNES & THORNBURG LLP, 11 SOUTH MERIDIAN, INDIANAPOLIS, IN, 46204, US

(21) Appl. No.: 12/300,951

(22) PCT Filed: May 16, 2007
(86) PCT No.: PCT/US07/11681

§ 371 Date: Nov. 14, 2008

Related U.S. Application Data
(60) Provisional application No. 60/801,130, filed on May 16, 2006.

(51) Int. Cl. A61F 2/00; C12N 5/06; A61K 35/12

(52) U.S. Cl. 424/423; 435/397; 424/484; 424/93.7

(57) ABSTRACT

Cell culture scaffolds presenting a more biologically relevant microenvironment are disclosed. More particularly, these cell culture scaffolds comprise three-dimensional matrices/biomaterials that are created from solubilized collagen compositions using controlled conditions to have the desired microstructure and mechanical properties. The engineered purified collagen based matrix compositions of the present invention can be used alone or in combination with cells as a tissue graft construct to enhance the repair of damaged or diseased tissues.

### CROSS-REFERENCE TO RELATED APPLICATIONS

This application claims the benefit, under 35 U.S.C. § 119(e), of U.S. provisional patent application Ser. No. 60/801,130 filed May 16, 2006 which is hereby incorporated by reference herein in its entirety.

### FIELD OF THE INVENTION

This invention relates to the preparation of collagen based matrices prepared from purified collagen compositions and their use as cell culture substrates and tissue graft constructs.

### BACKGROUND

The interaction of cells with their extracellular matrix (ECM) as it occurs in vivo plays a crucial role in the organization, homeostasis, and function of tissues and organs. Continuous communication between cells and their surrounding ECM environment orchestrates critical processes such as the acquisition and maintenance of differentiated phenotypes during embryogenesis, the development of form (morphogenesis), angiogenesis, wound healing, and even tumor metastasis. Both biochemical and biophysical signals from the ECM modulate fundamental cellular activities including adhesion, migration, proliferation, differential gene expression, and programmed cell death.

In turn, the cell can modify its ECM environment by modulating the synthesis and degradation of specific matrix components. The realization of the significance of cell-ECM interaction has led to a renewed interest in characterizing ECM constituents and the basic mechanisms of cell-ECM interaction.

Tissue culture allows the study in vitro of animal cell behavior in an investigator-controlled physiochemical environment. Presumably cultured cells function best (i.e., proliferate and perform their natural in vivo functions) when cultured on substrates that closely mimic their natural environment. Currently, studies in vitro of cellular function are limited by the availability of cell growth substrates that present the appropriate physiological environment for proliferation and development of the cultured cells. Complex scaffolds representing combinations of ECM components in a natural or processed form are commercially available, such as Human Extracellular Matrix (Becton Dickinson) and MATRIGEL®. However, none of the existing scaffolds have been prepared under conditions that regulate the polymerization of the scaffold in a controlled manner so as to produce a composition having mechanical properties and a predetermined 3D microstructure of collagen fibrils and/or soluble ECM components that optimizes cell substrate interactions to yield predictable and reproducible cellular outcomes. Applicants have discovered that the physical state of an ECM scaffold and not just its molecular composition should be considered in the design of new and improved scaffolds.

As reported herein, modifying the conditions used to form a collagen based matrix from a solubilized collagen solution allows for the controlled alteration of the micro-structural and subsequent mechanical properties of the resulting ECM scaffold. Furthermore, the micro-structural and mechanical properties of the ECM scaffold directly impact fundamental cell behaviors including survival, adhesion, proliferation, migration, and differentiation of cells cultured within the scaffold.

Because the molecular forces that orchestrate the self assembly of soluble, monomeric collagen into higher ordered structures are weak their assembly can easily turn into an unstructured aggregation of misfolded proteins. In the literature, there are known methods for isolating collagen from a variety of tissues, e.g., placenta and animal tails and using the isolated material to reconstitute collagenous matrices. These known methods rely on the protein's intrinsic ability to retain its secondary structure during protein isolation and assume that, for instance, the alpha helix will retain its helical structure throughout. The end result, even with a homogenous biochemical composition, can be a heterogeneous secondary structure. Controlling the assembly of the constituting monomers into tertiary or quaternary multimeric arrangements is very hard to achieve under such conditions. One embodiment of the present invention is directed to controlling the polymerization of a composition comprising solubilized collagen to form collagen fibrils and a collagen based scaffold that has the requisite microstructure and composition to allow for the expansion, differentiation and/or clonal isolation of cells in a highly reproducible and predictable manner.

### SUMMARY

The present invention relates to compositions comprising a three dimensional matrix that is formed to have the requisite composition, microstructure, and mechanical properties to enhance the proliferation and/or differentiation of cells, including stem cells or progenitor cells, cultured within such a matrix either in an in vitro or an in vivo setting.

In one embodiment the three dimensional matrix is prepared from a composition consisting of purified collagen, and in one embodiment the purified collagen is purified type I collagen or a mixture of purified type I and type III collagen. The three dimensional matrices typically have a fibril area fraction (defined as the percent area of the total area occupied by fibrils in a cross-sectional surface of the matrix; providing an estimate of fibril density)) of about 8% to about 26% and a elastic or linear modulus (stiffness; defined by the slope of the linear region of the stress-strain curve) of about 0.5 to about 40 kPa. The three dimensional matrix may further comprise a population of cells entrapped within the matrix. In one embodiment the three dimensional matrix is further provided with an exogenous source of glucose and calcium chloride.

In accordance with one embodiment, engineered purified collagen based matrices are used as novel compositions for inducing the repair of damaged or disease tissues in vivo. In one embodiment a tissue graft construct is provided comprising an engineered purified collagen based matrix, wherein the matrix is formed by contacting purified collagen with hydrochloric acid to produce a solubilized collagen composition and subsequently polymerizing the solubilized collagen composition under controlled conditions. In one embodiment the polymerization is conducted in the presence of a population of cells to produce the engineered purified collagen based matrix containing cells entrapped within the matrix.

In accordance with one embodiment, a composite tissue graft is provided. The composite tissue graft comprises a first three dimensional matrix and a second three dimensional matrix, wherein the first and second three dimensional matrices are physically connected to one another. The first and second three dimensional matrices differ in composition resulting in the two 3D matrices differing in fibril density, fibril dimensions, mechanical strength and stiffness, biochemical composition, including the content of collagenous and non-collagenous components, and the fluid composition of the matrix or a combination thereof. In one embodiment multiple pieces of a first three dimensional matrix are suspended within, and surrounded by, the second three dimensional matrix. In another embodiment at least two types of three dimensional matrices are constructed in a layered format. In a further embodiment one or both of the three dimensional matrices further comprise a population of cells, and in one embodiment the cells are stem cells.

### BRIEF DESCRIPTION OF THE DRAWINGS

FIGS. 1A-1G present data showing the effect of various parameters on the stiffness (elastic or linear modulus) of the formed matrix. FIG. 1A represents the effect of polymerization temperature on a matrix formed from a solubilized collagen composition comprising 1 mg/ml collagen in 1×PBS at pH 7.4; FIG. 1B represents the effect of the buffer type on a matrix formed from a solubilized collagen composition comprising 1 mg/ml collagen, and about 0.15 M NaCl at 37° C.; FIG. 1C represents the effect of pH (using a phosphate buffer) on a matrix formed from a solubilized collagen composition comprising 1 mg/ml collagen, in 1×PBS at pH 7.4; FIG. 1D represents the effect of pH (using a tris buffer) on a matrix formed from a solubilized collagen composition comprising 1 mg/ml collagen, in 50 mM tris, and about 0.15 M NaCl at 37° C.; FIG. 1E represents the effect of ionic strength on a matrix formed from a solubilized collagen composition comprising 1 mg/ml collagen, no buffer, at 37° C.; FIG. 1F represents the effect of phosphate concentration on a matrix formed from a solubilized collagen composition comprising 1 mg/ml collagen, and about 0.15 M NaCl at 37° C.; FIG. 1G represents the effect of SIS component concentration on a matrix formed from a solubilized ECM collagen composition in 1×PBS at 37° C.

FIGS. 2A & 2B represent a series of graphs showing the quantification of fibril area fraction (FIG. 2A) and fibril diameter distribution (FIG. 2B) based upon confocal and SEM images, respectively. All fibril area fraction relationships showing statistically significant differences (p<0.05) are indicated with symbols (*,**,,◯).

FIGS. 3A-3D represent a series of graphs showing cell length (FIG. 3A), width (FIG. 3B), length/width ratio (FIG. 3C), and surface area (FIG. 3D) determined and compared for neonatal human dermal fibroblasts (NHDFs) seeded within 3D ECMs prepared with 1.5 mg/ml type I collagen, and a type III collagen content that varied from 0 to 0.75 mg/ml. Results represent the means and standard deviations for 10≦n≦23 cells analyzed for each ECM formulation at a given time point. All groups showing statistically significant differences (p<0.05) are marked with the same symbol.

FIGS. 4A-4D represent a series of images depicting cell contractility and matrix remodeling by individual NHDFs resident within type I collagen (1.5 mg/ml) ECMs prepared with type III collagen concentrations of 0.25 mg/ml (FIGS. 4A and 4B) and 0.75 mg/ml (FIGS. 4C and 4D). FIGS. 4A and 4C represent 2D projections of confocal reflection image stacks showing changes to NHDF morphology and collagen fibril microstructure observed 5 hours after polymerization. FIGS. 4B and 4D represent quantified levels of local volumetric strain (matrix deformation) within the 3D tissue construct.

FIG. 5 represents a graph depicting contractility and matrix remodeling within engineered ECMs. NHDFs were grown within engineered ECMs in which the type I collagen concentration was kept constant at 1.5 mg/ml and the amount of type III collagen was either 0.25 mg/ml or 0.75 mg/ml. Average local 3D principal strains for a single cell and its surrounding ECM were quantified 5 hours post-polymerization (5≦n≦6). Negative strain values indicate compressive deformations. All relationships showing statistically significant differences (p<0.05) are indicated with symbols (*,**,,◯,□,+).

FIG. 6 represents a graph showing that points of maximum local deformation or strain induced within a 3D tissue construct by low passage neonatal human dermal fibroblasts occurred at distances further from the cell than for engineered ECMS prepared with lower amounts of type III collagen. NHDFs were grown within engineered ECMs in which the type I collagen concentration was kept constant at 1.5 mg/ml and the amount of type III collagen was either 0.25 mg/ml or 0.75 mg/ml.

FIGS. 7A & 7B represent a series of graphs depicting data regarding the proliferation of low passage human dermal fibroblasts when grown within a 3D ECM format consisting of type I collagen ECMs prepared within increasing amounts of type III collagen (see FIG. 7A). NHDFs exposed to 2D ECM surface coatings representing the same biochemical compositions and collagen type I/III ratios showed no significant changes in proliferative response (see FIG. 7B). Selected relationships showing statistically significant differences (p<0.05) are indicated with symbols (*, **,,◯).

FIG. 8 represents a bar chart showing differences in the expression of select tissue-specific genes by multi-potential bone marrow derived mesenchymal cells grown on standard 2D plastic and within 3D ECM microenvironments of increased fibril density and stiffness (elastic or linear modulus). Gene expression patterns for mesenchymal cells cultured within a given 2D or 3D format was also modulated by changing the composition of the culture medium.

FIG. 9 is a schematic representation of general cell behavior of multi-potential bone marrow derived mesenchymal cells when cultured within 3D matrices that differ in collagen concentration to provide an ECM microenvironment characterized by increased fibril density and stiffness (elastic or linear modulus). Points of arrow indicate low frequency events and wide ends of arrows indicate high frequency events.

### DETAILED DESCRIPTION

### Definitions

As used herein, the term “stem cell” refers to an unspecialized cell from an embryo, fetus, or adult that is capable of self-replication or self-renewal and can develop into specialized cell types of a variety of tissues and organs. The term as used herein, unless further specified, encompasses totipotent cells (those cells having the capacity to differentiate into extra-embryonic membranes and tissues, the embryo, and all post-embryonic tissues and organs), pluripotent cells (those cells that can differentiate into cells derived from any of the three germ layers) and multipotent cells (those cells having the capacity to differentiate into a limited range of differentiated cell types).

As used herein the term “progenitor cell” refers to a stem cell with more specialization and less differentiation potential than a totipotent stem cell. For example, progenitor cells include unipotential cells (those cells having the capacity to differentiate along a single cell lineage).

As used herein, the term “lyophilized” relates to the removal of water from a composition, typically by freeze-drying under a vacuum. However, lyophilization can be performed by any method known to the skilled artisan and the method is not limited to freeze-drying under a vacuum. Typically, the lyophilized tissue is lyophilized to dryness, and in one embodiment the water content of the lyophilized tissue is below detectable levels.

As used herein “solubilized collagen composition” refers to a composition that comprises collagen in a predominantly soluble monomeric form (i.e., wherein less than 20% of the collagen is insoluble, denatured, or assembled in higher ordered structures).

As used herein “solubilized extracellular matrix composition” refers to a naturally occurring extracellular matrix that has been treated, for example, with an acid to reduce the molecular weight of at least some of the components of the extracellular matrix and to produce a composition wherein at least some of the components of the extracellular matrix have been solubilized from the extracellular matrix. The “solubilized extracellular matrix composition” may include insoluble components of the extracellular matrix as well as solubilized components.

As used herein the term “collagen-based matrix” refers to an extracellular matrix that comprises collagen. An “engineered purified collagen based matrix” as used herein relates to a composition comprising a collagen fibril scaffold that has been formed under controlled conditions from a solubilized collagen composition, wherein the solubilized collagen composition is prepared from a composition consisting essentially of collagen. The conditions controlled during the polymerization reaction include one or more of the following: pH, phosphate concentration, temperature, buffer composition, ionic strength, and composition and concentration of purified collagen components. Similarly, an “engineered extracellular matrix” relates to a solubilized extracellular matrix composition that is polymerized to form a collagen fibril matrix under controlled conditions, wherein the controlled conditions include pH, phosphate concentration, temperature, buffer composition, ionic strength, and composition and concentration of the extracellular matrix components which includes both collagen and non-collagenous molecules. A “bioactive engineered extracellular matrix” composition refers to an engineered extracellular matrix composition that can be polymerized to form a three dimensional scaffold that is capable of remodeling tissues in vivo.

As used herein the term “naturally occurring extracellular matrix” comprises any noncellular material naturally secreted by cells (such as intestinal submucosa) isolated in their native configuration with or without naturally associated cells.

As used herein the term “submucosal matrices” refers to natural extracellular matrices, known to be effective for tissue remodeling, that have been isolated in their native configuration, including submucosa derived from vertebrate intestinal tissue, stomach tissue, bladder tissue, alimentary tissue, respiratory tissue and genital tissue.

As used herein the term “exogenous” or “exogenously added” designates the addition of a new component to a composition, or the supplementation of an existing component already present in the composition, using material from a source external to the composition.

As used herein “sterilization” or “sterilize” or “sterilized” means removing unwanted contaminants including, but not limited to, endotoxins, nucleic acid contaminants, and infectious agents.

As used herein “stiffness” or elastic or linear modulus” refers to the fundamental material property defined by the slope linear portion of a stress-strain curve that results from conventional mechanical testing protocols.

As used herein, the term “purified” and like terms relate to the isolation of a molecule or compound in a form that is substantially free from other components with which they are naturally associated (e.g., the total amount of nondesignated components present in the composition representing less than 5%, or more typically less than 1%, of total dry weight).

As used herein the term “three dimensional purified collagen matrix (3D matrix)” refers to an engineered purified collagen based matrix, as defined above, and the fluid that surrounds the collagen fibril network. A “3D purified collagen matrix populated/seeded with cells” further comprises a viable population of cells contained within the matrix.

As used herein the term “three dimensional extracellular matrix (3D ECM)” refers to an engineered extracellular matrix, as defined above, and the fluid that surrounds the collagen fibril network. A “3D extracellular matrix populated/seeded with cells” further comprises a viable population of cells contained within the matrix.

As used herein the term “three dimensional matrix (3D matrix)” is a generic term that is intended to include both “three dimensional purified collagen matrices (3D purified collagen matrices)” as well as “three dimensional extracellular matrices (3D ECM)

As used herein the term “collagen fibril” refers to a quasi-crystalline, filamentous structure formed by the self-assembly of soluble trimeric collagen molecules. The collagen molecules in a collagen fibril typically pack in a quarter-staggered pattern giving the fibril a characteristic striated appearance or banding pattern along its axis. Solubilized collagen that is assembled in vitro to form collagen fibrils exhibit similarities to collagen structures found in vivo (Veis and George, 1994 Fundamental of interstitial collagen assembly. In: Yurchenco P D, Birk D E, and Mecham R P (eds.), Extracellular Matrix Assembly and Structure, Academic Press, Inc., San Diego, pp. 15-45.). Within tissues in vivo, collagen fibrils are organized as bundles in a hierarchical manner to form fibers. Collagen fibers are further organized in a tissue-specific fashion to provide tissues with specific structural-functional properties. Collagen fibrils are distinct from the amorphous aggregates or precipitates of insoluble collagen that can be formed by dehydrating (e.g., lyophilization) collagen suspensions to form porous network scaffolds. Collagen networks formed from amorphous aggregates, or precipitates of insoluble collagen, have characteristics that distinct from those formed from collagen fibrils as defined above.

### EMBODIMENTS

Cell culture scaffolds presenting a more biologically relevant microenvironment are disclosed. More particularly, these cell culture scaffolds comprise three-dimensional matrices/biomaterials that are created from solubilized collagen compositions. The solubilized collagen compositions are prepared from biological sources, such as naturally occurring extracellular matrices, including for example submucosal matrices. More particularly, the soluble polymers suitable for use in the present invention can be isolated, to varying degrees of purity, from natural tissues and include, but are not limited to, type I collagen, type III collagen, growth factors and glycosaminoglycans. In one embodiment the solubilized collagen composition comprises purified type I collagen or a mixture of purified type I and type III collagen. When provided with the proper conditions, the solubilized collagen composition undergoes polymerization/self assembly to form a three dimensional scaffold/biomaterial comprised of collagen fibrils. In one embodiment the soluble polymers of the solubilized collagen composition comprise type I collagen monomers, where upon polymerization the resulting scaffolds represent a composite material, comprising insoluble collagen fibrils and an interfibrillar fluid component that is referred to herein as a three dimensional matrix.

An array of scaffolds/biomaterials can be created by varying the composition of ECM molecules as well as the self-assembly/polymerization conditions. Surprisingly, applicants have discovered that upon seeding progenitor cells or stem cells within engineered collagen based matrices (scaffolds) representing different microstructural compositions (e.g., having different dimensioned and organization of the collagen fibrils and filaments), distinct patterns of cell survival, growth, proliferation and differentiation are obtained. In particular, applicants have discovered that seeding of stem cells within engineered collagen based matrices representing different microstructural compositions (e.g., varied fibril dimensions (length, diameter) and densities) impacts the rate of cell proliferation as well as the pattern of cellular condensation, aggregation, fusion and cellular differentiation events and their associated time-line. These results are significant because they indicate that engineered purified collagen based matrices can be specifically designed to foster the proliferation of stem cells while maintaining their precursor or multi-potential status. Furthermore, engineered purified collagen based matrices can be designed to direct differentiation of cells down a specific cell lineage or maintain cells in their differentiated state (such as fat, bone, muscle, or cartilage) to form 3D organotypic tissues (that is reminiscent of in vivo tissue structure and function).

In accordance with one embodiment, the collagen component of the solubilized collagen composition consists essentially of purified collagen, the majority of which are in monomeric form. In one embodiment collagen, and more particularly type I or type III collagen, that has been isolated from tissues is subjected to a final purification step that removes any reagents that were used during the isolation steps. In one embodiment the final purification step comprises dialyzing the isolated collagen in an aqueous solution, and in one embodiment the isolated collagen is dialyzed against a diluteacid solution, including for example, hydrochloric acid. In one embodiment the final purification step comprises dialyzing the isolated collagen against a 0.01 N HCl solution.

In one embodiment the composition is formed from purified collagen (the majority of which are in monomeric form) that is greater than 75% type I collagen, or greater than 90% type I collagen. In accordance with one embodiment the solubilized collagen composition is prepared using purified type I collagen as a starting material. Isolated type I or isolated type m collagen preparations are commercially available, and these commercially available materials are subjected to a further purification step, including for example, dialyzing against a dilute acid (including for example about 0.001 N to about 0.1 N hydrochloric acid solution, to produce purified collagen suitable for use for forming 3D purified collagen matrices. The dialysate can optionally be filtered and/or centrifuged to remove particulate matter to prepare a purified collagen composition for forming the 3D matrices. In one embodiment a composition consisting essentially of purified collagen is dissolved in an acid solution, such as hydrochloric acid to prepare a solubilized collagen composition of the desired concentration. In one embodiment the purified collagen is dissolved in about 0.001 N to about 0.1 N, from about 0.005 N to about 0.1 N, from about 0.005 N to about 0.01 N, from about 0.01 N to about 0.1 N, from about 0.05 N to about 0.1 N, from about 0.001 N to about 0.05 N, about 0.001 N to about 0.01 N, or from about 0.01 N to about 0.05 N hydrochloric acid solution.

In another embodiment, a three dimensional purified collagen matrix is provided, wherein the matrix is formed from a solubilized collagen composition wherein the collagen components of the solubilized collagen composition consist essentially of purified type I and type III collagen. The component fibrils of such matrices have been found to have a greater degree of flexibility relative to the fibrils of engineered purified collagen matrices that are formed using only type I collagen, when equivalent total amounts of collagen are used to form the respective matrices. In one embodiment the matrix comprises type I collagen and type III collagen in a ratio of 200:1. The method of forming matrices with fibrils that exhibit a higher degree of flexibility comprises the steps of combining in vitro at least 100 ug/ml of type I collagen with at least 0.5 ug/ml of type III collagen to obtain a total amount of collagen, and forming in vitro a three dimensional purified collagen matrix wherein the three dimensional matrix has decreased stiffness compared to a 3D matrix formed in vitro with type I collagen when the total amount of collagen in the two matrices is equivalent.

In another embodiment, a method of preparing an extracellular matrix composition is provided. The method comprises the steps of combining in vitro at least 100 ug/ml of type I collagen with at least 0.5 ug/ml of type III collagen to obtain a total amount of collagen, and forming in vitro a three dimensional matrix. In one embodiment the type I and type III collagen is dissolved in about 0.001 N to about 0.1 N, from about 0.005 N to about 0.1 N, from about 0.005 N to about 0.01 N, from about 0.01 N to about 0.1 N, from about 0.05 N to about 0.1 N, from about 0.001 N to about 0.05 N, about 0.001 N to about 0.01 N, or from about 0.01 N to about 0.05 N hydrochloric acid solution either before or after the combining step.

In another embodiment, an extracellular matrix composition for use in repairing diseased or damaged tissues is provided. The extracellular matrix composition comprises at least 100 ug/ml of type I collagen and at least 0.5 ug/ml of type III collagen, wherein the type I collagen to type III collagen ratio is selected from the group consisting of 200:1, 100:1, 50:1, 15:1, 10:1, 8:1, 6:1, 5:1, 3:1, and 2:1, and a population of cells. The matrix is formed by provided a solubilized collagen composition comprising type I and type III collagen, in a ratio selected from the group consisting of 200:1, 100:1, 50:1, 15:1, 10:1, 8:1, 6:1, 5:1, 3:1, and 2:1, polymerizing the solubilized collagen composition to form collagen fibrils. In accordance with one embodiment cells are added to the collagen composition either before or after the polymerization step. Cells can be seeded at a relatively high density of about 1×10⁶ to about 1×10⁸ cells/ml, or at a more typical density of about 1×10³ to about 1×10⁵ cells/ml. Seeding the cells at the relative high density of about 1×10⁶ to about 1×10⁸ cells/ml will promote cell to cell interactions over cell to matrix interactions. Accordingly, stem cells seeded at relatively high densities will develop into fat tissue even when the cells are cultured within 3D matrices of high collagen fibril density. In one embodiment stem cells are seeded at a density of less than 5×10⁴ cells/ml, more typically at a density of about 5×10⁴ cells/ml. In another embodiment stem cells are seeded at a density of less than 1×10⁴ cells/ml, in another embodiment stem cells are seeded at a density selected from a range of about 1×10² to about 5×10³.

In one embodiment a composition comprising solubilized collagen, or a composition comprising solubilized collagen and cells is injected into a host and the polymerization of the solubilized collagen composition occurs in vivo to form a three dimensional matrix. Alternatively, the solubilized collagen composition can be polymerized in vitro and the polymerized matrix, comprising the population of cells, can be subsequently injected or implanted in a host. In another embodiment the population of cells entrapped within the 3D matrix can be cultured in vitro, for a predetermined length of time, to increase cell numbers and/or induce differentiation of the cell population prior to implantation into a host. In a further embodiment, the population of cells can be cultured in vitro, for a predetermined length of time, to increase cell numbers and/or induce differentiation of the cell population and the cells can be separated from the matrix and implanted into the host in the absence of the polymerized matrix.

In one illustrative embodiment, the engineered purified collagen based matrix comprises type III collagen in the range of about 0.5% to about 33% of total collagen in the matrix. In another illustrative embodiment, the engineered purified collagen based matrix comprises type I collagen in the range of about 66% to about 99.5% of total collagen in the matrix. In yet another illustrative embodiment, the type I collagen to type III collagen ratio is in the range of about 2:1 to about 200:1, wherein the type I collagen to type III collagen ratio may be selected from the group consisting of 200:1, 100:1, 50:1, 15:1, 10:1, 8:1, 6:1, 5:1, 3:1, and 2:1.

In another embodiment, a method of enhancing cell proliferation within an extracellular matrix composition is provided. The method comprises the steps of combining in vitro an amount of type I collagen with an amount of type III collagen to obtain a total amount of collagen wherein the ratio of type III collagen to type I collagen is at least 1:6, and forming in vitro a three-dimensional extracellular matrix wherein the extracellular matrix enhances cell proliferation compared to an extracellular matrix formed in vitro with type I collagen wherein the amount of type I collagen is equivalent to the total amount of type I collagen in the combining step. In yet another embodiment, the method comprises the steps of combining in vitro at least 3 ug/ml of type I collagen with at least 0.5 ug/ml of type III collagen to obtain a total amount of collagen wherein the ratio of type III collagen to type I collagen is at least 1:6, and forming in vitro a three-dimensional extracellular matrix wherein the extracellular matrix enhances cell proliferation compared to an extracellular matrix formed in vitro with type I collagen, wherein the amount of type I collagen is equivalent to the total amount of type I collagen in the combining step.

In another illustrative embodiment, the method of preparing an engineered purified collagen based matrix comprises combining type I and type III collagen wherein the type III collagen is added in the range of about 17% to about 33% of total collagen in the matrix. In another illustrative embodiment, the type I collagen is added in the range of about 66% to about 83% of total collagen in the matrix. In yet another illustrative embodiment, the type I collagen to type III collagen ratio is in the range of about 6:1 to about 1:1, wherein the type I collagen to type III collagen ratio may be selected from the group consisting of 6:1, 5:1, 4:1, 3:1, 2:1, and 1:1.

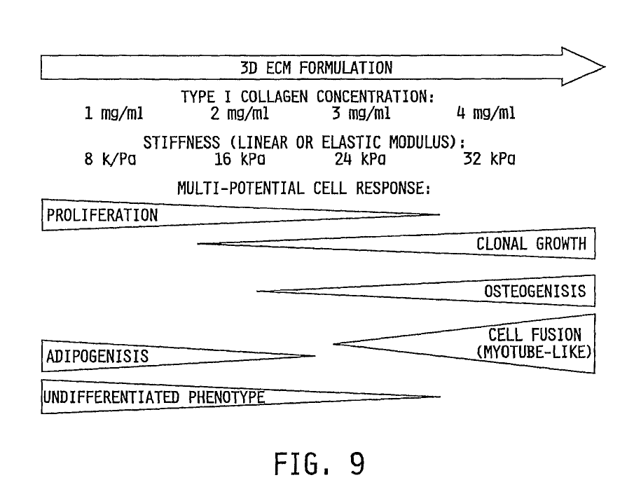

Applicants have also discovered that the concentration of total collagen present in solubilized collagen composition will impact the microstructure of the matrix, and the behavior of stem cells cultured within a matrix polymerized from such a composition (see FIG. 9). 3D matrices can be prepared from solubilized collagen compositions having purified collagen concentrations ranging from as little as 0.05 mg/ml to as much as 40 mg/ml. Typically the 3D matrices are prepared from purified solubilized collagen compositions having a collagen concentration selected from a range of about 0.1 mg/ml to about 5.0 mg/ml, and in one embodiment about 1.5 mg/ml to about 3.0 mg/ml. Table 1 summarizes the effect of total collagen concentration on the fibril structure of the matrix:

TABLE 1
Microstructure and Mechanical Properties of 3D Purified Collagen Matrices

| Collagen | Fibril Area | Stiffness | Fibril Diameter | Fibril Diameter |
|---|---|---|---|---|
| Concentration | Fraction | (Linear | (confocal reflection | (scanning electron |
| (mg/ml) | (Density; %) | Modulus; kPa) | microscopy; nm) | microscopy, nm) |
| 0.3, pH 7.4 | | 1.54 ± 0.507 | 418 ± 121 | |
| 1, pH 7.4 | 11.5 ± 1.9 | 10.7 ± 1.93 | 446 ± 65 | |
| 1.5, pH 7.4 | 12 ± 1.4 | 8.5 ± 1.65 | 412.63 ± 76 | 115.16 ± 23.18 |
| 2, pH 7.4 | 14.8 ± 4.25 | 16.6 ± 2.68 | 435 ± 61 | 80.8 ± 18.3 |
| 3, pH 7.4 | 18.4 ± 1.9 | 24.3 ± 4.16 | 430 ± 71 | |
| 2, pH 6 | | 1.84 ± 0.701 | 490 ± 96 | |
| 2, pH 7 | | 12.7 ± 1.18 | 469 ± 73 | |
| 2, pH 7.4 | | 16.6 ± 2.68 | 435 ± 61 | |
| 2, pH 8 | | 22.5 ± 3.65 | 421 ± 62 | |
| 2, pH 9 | | 33.0 ± 6.93 | 392 ± 65 | |
| 1.5 I + 0.75 III | 21.5 ± 2.6 | 13.3 ± 1.4 | 385 ± 72 | 87 ± 17 |

Using the data of Table 1 and assuming a linear relationship between collagen concentration and the measure properties, predictions of fibril area fraction and matrix stiffness can be determined as a function of collagen concentration using the following equations:

Fibril Area Fraction=3.6514*Collagen Concentration+7.3286

R²=0.9681

Stiffness=8.1145*Collagen Concentration−0.3306

R²=0.9304

Prediction of Stiffness as a function of Fibril Diameter (Assumption: fibril area fraction does not change; relationship based upon pH data):

Stiffness=−0.2916 Fibril Diameter+146.02

R²=0.9581 (based upon pH data)

The 3D matrices formed in accordance with the present disclosure represent a matrix of collagen fibrils. The fibrils of the matrices are formed at a fibril area fraction (density) of about 7.7% to about 25% total volume. In one embodiment the 3D matrices have a fibril area fraction of about 12.8% to about 18.3% total volume. In another embodiment the 3D matrices have a fibril area fraction of about 18.5% to about 25% total volume. Three dimensional matrices having low fibril density and low stiffness enhance stem cell proliferation with decreased differentiation of the cells. Accordingly, 3D matrices formed from solubilized collagen compositions having about 0.1 mg/ml to about 3 mg/ml collagen, and more typically about 0.5 mg/ml to about 2.5 mg/ml collagen are utilized to stimulate stem cell proliferation. The 3D matrices so formed will have a fibril predicted fibril area fraction (density) of about 7.7% to about 18.3% total volume and about 9.2% to about 16.5% total volume, respectively. In one embodiment the 3D matrices are formed from solubilized collagen compositions having about 3 mg/ml to about 1.5 mg/ml collagen and in one embodiment the solubilized collagen compositions have about 2.5, 2.0, 1.5, or 1.0 mg/ml of collagen.

Alternatively, higher concentrations of total collagen present in the three dimensional matrix leads to differentiation of stem cells. Accordingly, 3D matrices (having a fibril area fraction of at least about 18% total volume) formed from solubilized collagen compositions having more than about 3 mg/ml are utilized to stimulate differentiation of stem cells cultured within the matrix. In one embodiment the 3D matrices are formed from solubilized collagen compositions having about 3.2, 3.4, 3.6, 3.8, 4.0, 4.5 or 5.0 mg/ml of collagen, resulting in 3D matrices having a fibril area fraction of about 19%, 19.7%, 20.5%, 21.2%, 22%, 23.8% and 25.6% total volume, respectively.

As reported herein the relative stiffness (tensile strength) of a 3D matrix can be modified by controlling the relative proportion of type I to type III collagen, the fibril area fraction number (density), or the fibril diameter of the collagen fibrils in the 3D matrix. In accordance with one embodiment 3D matrices are prepared having a relatively low stiffness (tensile strength) of about 0.48 to about 24.0 kPa. In one embodiment these matrices are used to propagate stem cells and progenitor cells without further differentiation of the cells and/or their progeny. In another embodiment 3D matrices are prepared having a relatively high stiffness of about 25 to about 40 kPa. In one embodiment these relatively stiffer matrices are used to induce the differentiation of stem cells and progenitor cells and/or their progeny. In one embodiment a 3D matrix is provided having a relatively low stiffness of about 0.48 to about 24.0 kPa and a relatively low fibril area fraction (density) of about 7% to about 18% total volume. In an alternative embodiment a 3D matrix is provided having a relatively high stiffness of about 25 to about 40 kPa and a relatively high fibril area fraction (density) of about 19% to about 26% total volume.

In accordance with one embodiment a composite tissue construct is prepared wherein the composite comprises a first 3D matrix and a second 3D matrix. The first and second 3D matrices are in physical contact or are physically connected with one another, wherein the composition of the first 3D matrix and the second 3D matrix differ from on another by at least one element. The first and second matrices may differ from one another based on any of the components of the 3D matrix (e.g., concentration of fibrils, type of collagen comprising the matrix, the composition and concentration of the components comprising the fluid, etc). In accordance with one embodiment the first and second matrices differ from each other based on the stiffness of the respective matrices. In one embodiment the first matrix has a different fibril content, fibril dimensions, biochemical composition including content of collagenous (e.g., a different type I collagen to type III collagen ratio) and non-collagenous components, interfibrillar fluid properties or combination thereof relative to the second matrix.

In one embodiment the composite tissue graft construct comprises a first 3D purified collagen matrix and a second 3D purified collagen matrix, or alternatively, a composite of a first 3D extracellular matrix and a second 3D extracellular matrix, or combination of a 3D purified collagen matrix with a 3D extracellular matrix. In one embodiment at least one of the first and second 3D matrices further comprises a population of cells, including for example a population of stem cells. Alternatively, both the first and second 3D matrices may comprise a population of cells. When both matrices of the composite tissue graft comprise a population of cells, the cells of the first 3D matrix may be the same or different than the cells contained in the second 3D matrix. Accordingly, in one embodiment the first and second matrices only differ from each other based on the type of cells populating each respective matrix. In one embodiment the three dimensional matrix comprising the population of cells further comprises exogenously added glucose and calcium chloride.

In one embodiment the composite tissue graft comprises a laminate structure that includes at least one first 3D matrix layered onto or polymerized on top of a second 3D matrix. In one embodiment the composite tissue graft comprises multiple sheets of a first 3D matrix layered and second 3D matrix layered on top of one another in an alternating pattern. In one embodiment the laminate structure comprises three layers, with the first 3D matrix layer sandwiched between two layers of the second 3D matrix. The layered composite tissue graft can be further provided with a population of cells. More particularly, one of the first or second three dimensional matrices may further comprise a population of cells entrapped within the first or second three dimensional matrix, and in one embodiment the first 3D matrix comprises a population of stem cells. The sheets of the laminate composite tissue graft can be held together using standard techniques known to those skilled in the art, including but not limited to the use of sutures, clips, staples, fasteners, screws or by fusing the layers together by applying pressure under dehydrating conditions. Procedures for preparing multilayered collagen constructs have been described in U.S. Pat. Nos. 6,666,892; 5,992,028; 5,955,110; and 5,885,619, the disclosures of which are incorporated herein. The sutures, clips, staples, fasteners, screws or other fastening devices in one embodiment can be prepared from biodegradable materials as is known in the art.

In one embodiment the first and second 3D matrices are physically connected to one another by contacting a first 3D matrix with a solubilized collagen composition and polymerizing the solubilized collagen composition to form the second 3D matrix. Polymerization of the second 3D matrix while maintaining contact with the first 3D matrix will result in intertwining of the fibrils from the first 3D matrix with the fibrils of the second 3D matrix, and thus physically connect the first and second 3D matrices. In this embodiment no further fastening devices are required to form the composite structure. In a further embodiment a step-wise polymerization procedure can be used to form a multi-layered 3D matrix construct, wherein layer 1 is polymerized and then layer 2 is polymerized on top of that, the steps being repeated until the number of desired layers has been achieved.

In an alternative embodiment the first matrix may be embedded within the second matrix. In accordance with one embodiment, the first 3D matrix is provided in particulate form. The individual particles of the first 3D matrix can be of any shape, and in one embodiment the first 3D matrix is provided as a plurality of spherical shaped pieces of 3D matrix. The three dimensional matrix can be provided in particulate form either by fragmenting a larger piece of 3D matrix or forming the original 3D matrix as particles of the desired size and shape. In one embodiment the particulates have an average length of about 50 um to about 300 um, and in one embodiment about 200 um in their longest dimension. In one embodiment the particulates are approximately spherical in shape and have an average diameter of about 50 um to about 200 um, and in one embodiment about 100 um.

In one embodiment, the multiple pieces of the first three dimensional matrix are suspended in a solubilized collagen composition, wherein polymerization of the solubilized collagen composition forms the second three dimensional matrix, entrapping and surrounding the first three dimensional matrix pieces. In one embodiment a solubilized collagen composition comprising pieces of a first 3D matrix suspended therein is injected into a host and the polymerization of the solubilized collagen composition occurs in vivo to form the composite tissue graft. In an alternative embodiment the polymerization of the solubilized collagen composition comprising suspended pieces of the 3D matrix is conducted in vitro. The composite tissue graft formed in vitro can then be used as an in vitro cell culture substrate or the tissue graft construct can be implanted into a host, either directly after formation of the construct, or after culturing the matrix in vitro with cells for a predetermined length of time. In one embodiment the embedded first 3D matrix has decreased fibril density and stiffness relative to the second matrix. In one embodiment the first matrix has a lower ratio of type I to type III collagen, or has a lower concentration of total collagen fibrils, or both, relative to the collagen present in the second matrix.

In one embodiment one of the first or second three dimensional matrices of the composite tissue graft further comprises a population of cells entrapped within the respective three dimensional matrix. In another embodiment the first three dimensional matrix, and optionally the second three dimensional matrix as well, further comprises a population of cells entrapped within their respective matrices. In one embodiment the cells entrapped within the first three dimensional matrix are stem cells. When cells are included in the composite tissue graft, the tissue graft may further comprises exogenously added glucose and calcium chloride.

In accordance with one embodiment a composite tissue graft is provided comprising a first 3D matrix embedded within a second 3D matrix, wherein the first 3D matrix has an elastic or linear modulus (stiffness) of 0.48 to about 24.0 kPa, and the second 3D matrix has an elastic or linear modulus (stiffness) of about 25 to about 40 kPa. In accordance with one embodiment a composite tissue graft is provided comprising a first 3D matrix and a second 3D matrix wherein the first 3D matrix has a fibril area fraction of about 7% to about 18% and a tensile strength of 0.48 to about 24.0 kPa, and the second 3D matrix has a fibril area fraction of about 19% to about 26% and a tensile strength of about 25 to about 40 kPa. In a further embodiment, this composite tissue graft further comprises a population of stem cells or progenitor cells embedded within the matrix of the first 3D matrix.

In accordance with one embodiment a composite graft is provided comprising a first 3D matrix embedded within a second 3D matrix, wherein the first and second 3D matrices each represent a 3D purified collagen matrix. Alternatively, in one embodiment one of said first and second 3D matrices represents a 3D purified collagen matrix and the other represents a 3D extracellular matrix. In another embodiment the first and second 3D matrices each represent a 3D extracellular matrix.

In accordance with one embodiment the 3D matrices can be further processed by lyophilizing or crosslinking the formed 3D matrix. In accordance with one embodiment a composite construct can be formed comprising a first 3D matrix that is embedded or enclosed by a second 3D matrix wherein the first 3D matrix has been lyophilized or crosslinked. Crosslinking of the 3D matrices can be accomplished using standard reagents (such as gluteraldehyde) known to those skilled in the art. In one embodiment the composite construct comprises a first inner 3D matrix that has been crosslinked and is embedded within a second 3D matrix wherein the second 3D matrix further comprises a cell population suspended within the second 3D matrix.

In one embodiment a composite tissue graft composition is provided having a first 3D matrix comprising purified type I collagen and purified type III collagen wherein the type III collagen is present at a concentration about 17% to about 33% of total collagen in the first 3D matrix. The second 3D matrix may comprise only purified type I collagen as the collagen component of the matrix, or alternatively the matrix may comprise purified type I collagen and purified type III collagen, wherein the amount of type III collagen when present in the second 3D matrix is less than about 17% of the total collagen in the second 3D matrix. In one embodiment the purified collagen used to form the 3D matrices of the composite tissue constructs is first treated/suspended in about 0.001 N to about 0.1 N, from about 0.005 N to about 0.1 N, from about 0.005 N to about 0.01 N, from about 0.01 N to about 0.1 N, from about 0.05 N to about 0.1 N, from about 0.001 N to about 0.05 N, about 0.001 N to about 0.01 N, or from about 0.01 N to about 0.05 N hydrochloric acid solution. In one embodiment the cells contained within the composite tissue graft are stem cells or progenitor cells.

In another embodiment the solubilized collagen composition used to form the three dimensional matrices comprises collagen monomers isolated from natural tissues, and includes additional components that are naturally associated with the native tissues and/or exogenously added components. In one embodiment various exogenous materials, such as growth factors are added to the collagen based matrices of the present invention. In one embodiment the solubilized collagen composition represents a solubilized fraction of a naturally occurring extracellular matrix, and in one embodiment the naturally occurring extracellular matrix is a vertebrate submucosal matrix. In one embodiment the solubilized collagen composition represents a solubilized fraction of vertebrate intestinal submucosa.

In other embodiments, acetic acid, formic acid, lactic acid, citric acid, sulfuric acid, ethanoic acid, carbonic acid, nitric acid, or phosphoric acid can be used to solubilize the naturally occurring extracellular matrix to produce a solubilized collagen composition. The solubilized collagen composition derived from a naturally occurring extracellular matrix, such as vertebrate intestinal submucosa, can then be polymerized to form an engineered extracellular matrix.

The invention also relates to methods of preparation and compositions comprising solubilized extracellular matrix components polymerized in vitro where the extracellular matrix components are solubilized by other methods known in the art. The polymerizing step can be performed under conditions that are systematically varied where the conditions are selected from the group consisting of pH, phosphate concentration, temperature, buffer composition, ionic strength, the extracellular matrix components in the solubilized extracellular matrix composition, and the concentration of the extracellular matrix components in the solubilized extracellular matrix composition.

In accordance with one embodiment a method of forming a 3D matrix comprising cells is provided. The method comprises the steps of providing an acid solubilized purified type I collagen composition. In one embodiment the collagen composition further comprises type III collagen. In one embodiment the purified collagen represents a commercially available isolated preparation of collagen that is further subjected to purification, including for example dialyzing against a solution of about 0.005 N to about 0.1 N HCl, more typically about 0.01 N HCl. Typically the solubilized collagen composition comprises purified collagen that is suspended in about 0.05 N to about 0.1 N HCl solution, and in one embodiment suspended in 0.01N HCl. The solubilized collagen composition is also typically sterilized using standard techniques including for example contact with chloroform or peracetic acid. Cells are then added to the solubilized collagen composition at a specific density. In one embodiment stem cells are added to the solubilized collagen composition at a concentration of about 10 to about 10⁸ cells/ml. In accordance with one embodiment the cells are stem cells, and in one embodiment, stem cells are added at a concentration of less than 5×10⁴ cells per milliliter, and more particularly in one embodiment stem cells are added at a density of about 10 to about 10³ per milliliter. In accordance with one embodiment the collagen/cell suspension is then pipetted into a well plate and allowed to polymerize in a humidified environment at 37° C. for approximately 30 minutes. In an alternative embodiment the collagen/cell suspension is injected into a host and the composition is polymerized in vivo.

As noted above solubilized collagen compositions can be prepared from purified collagen preparations or from vertebrate submucosal matrices, wherein in the later case, the collagen compositions comprise additional components besides collagen. Vertebrate submucosal matrices can be obtained from various sources, including intestinal tissue harvested from animals raised for meat production, including, for example, pigs, cattle and sheep or other warm-blooded vertebrates. According to one embodiment the solubilized collagen composition is derived from one or more sources selected from the group consisting of intestinal submucosa, stomach submucosa, urinary bladder submucosa, uterine submucosa, and any other submucosal material that can be used to remodel endogenous tissue.

In one embodiment the submucosa comprises the tunica submucosa delaminated from both the tunica muscularis and at least the luminal portion of the tunica mucosa of a warm-blooded vertebrate. Such constructs can be prepared by mechanically removing the luminal portion of the mucosa and the external muscle layers and lysing resident cells with hypotonic washes.

It is known that compositions comprising the tunica submucosa delaminated from both the tunica muscularis and at least the luminal portion of the tunica mucosa of the submucosal tissue of warm-blooded vertebrates can be used as tissue graft materials (see, for example, U.S. Pat. Nos. 4,902,508 and 5,281,422, the disclosures of which are incorporated herein by reference). Such submucosal tissue preparations are characterized by excellent mechanical properties, including high compliance, high tensile strength, a high burst pressure point, and tear-resistance.

Submucosa-derived matrices are collagen based biodegradable matrices comprising highly conserved collagens, glycoproteins, proteoglycans, and glycosaminoglycans in their natural configuration and natural concentration. Such submucosal material serves as a matrix for the regrowth of endogenous tissues at the implantation site (e.g., biological remodeling). The submucosal material serves as a rapidly vascularized matrix for support and growth of new endogenous connective tissue. Thus, submucosa matrices have been found to be trophic for host cells of tissues to which it is attached or otherwise associated in its implanted environment. In multiple experiments submucosal tissue has been found to be remodeled (resorbed and replaced with autogenous differentiated tissue) to assume the characterizing features of the tissue(s) with which it is associated at the site of implantation or insertion.

Small intestinal submucosa tissue is an illustrative source of submucosal tissue for use in this invention. Submucosal tissue can be obtained from various sources, for example, intestinal tissue can be harvested from animals raised for meat production, including, pigs, cattle and sheep or other warm-blooded vertebrates. Small intestinal submucosal tissue is a plentiful by-product of commercial meat production operations and is, thus, a low cost material.

The preparation of submucosal tissue is described in U.S. Pat. No. 4,902,508, the disclosure of which is expressly incorporated herein by reference. A segment of vertebrate intestine, for example, preferably harvested from porcine, ovine or bovine species, but not excluding other species, is subjected to abrasion using a longitudinal wiping motion to remove the outer layers, comprising smooth muscle tissues, and the innermost layer, i.e., the luminal portion of the tunica mucosa. The submucosal tissue is rinsed under hypotonic conditions, such as with water or with saline under hypotonic conditions, and is optionally sterilized.

The submucosal tissue can be sterilized using conventional sterilization techniques including glutaraldehyde tanning, formaldehyde tanning at acidic pH, propylene oxide or ethylene oxide treatment, gas plasma sterilization, gamma radiation, electron beam, and/or peracetic acid sterilization. Sterilization techniques which do not adversely affect the structure and biotropic properties of the submucosal tissue can be used. An illustrative sterilization technique is exposing the submucosal tissue to peracetic acid, 1-4 Mrads gamma irradiation (or 1-2.5 Mrads of gamma irradiation), ethylene oxide treatment, exposure to chloroform, or gas plasma sterilization. The submucosal tissue can be subjected to one or more sterilization processes. In illustrative embodiments, the intact extracellular matrix material can be sterilized with peracetic acid or the solubilized collagen composition can be sterilized. The submucosal tissue can be subjected to one or more sterilization processes, The submucosal tissue can be stored in a hydrated or dehydrated state prior to solubilization in accordance with the invention.

Extracellular matrix-derived tissues other than intestinal submucosa tissue may be used in accordance with the methods described herein and used as a source for preparing solubilized collagen compositions. Methods of preparing and treating other extracellular matrix-derived tissues are known to those skilled in the art and may be similar to the methods described above. For example, see WO 01/45765 and U.S. Pat. Nos. 5,163,955 (pericardial tissue); 5,554,389 (urinary bladder submucosa tissue); 6,099,567 (stomach submucosa tissues); 6,576,265 (extracellular matrix tissues generally); and 6,793,939 (liver basement membrane tissues); U.S. patent application publication no. US 2005/0019419-A1 (liver basement membrane tissues); and WO 2001/045765 (extracellular matrix tissues generally, each incorporated herein by reference. The preparation and use of submucosa tissues as graft compositions is also described in U.S. Pat. Nos. 4,902,508; 5,281,422; and 5,275,826, each incorporated herein by reference.

In one illustrative embodiment, the extracellular matrix material is solubilized with an acid and the solubilized fraction is recovered for polymerization to form the collagen based matrices of the present invention. Typically, prior to solubilization, the source extracellular matrix material is comminuted by tearing, cutting, grinding, or shearing the harvested extracellular matrix material. In one illustrative embodiment, the extracellular matrix material can be comminuted by shearing in a high-speed blender, or by grinding the extracellular matrix material in a frozen or freeze-dried state, and then lyophilizing the material to produce a powder having particles ranging in size from about 0.1 mm² to about 1.0 mm². The extracellular matrix material powder can thereafter be hydrated with, for example, water or buffered saline to form a fluid or liquid or paste-like consistency. In one illustrative embodiment, the extracellular matrix tissue is comminuted by freezing and pulverizing under liquid nitrogen in an industrial blender. The preparation of fluidized forms of the source extracellular matrix material, such as submucosa tissue, is described in U.S. Pat. No. 5,275,826, the disclosure of which is expressly incorporated herein by reference.

In one illustrative embodiment, an acid, such as hydrochloric acid, acetic acid, formic acid, sulfuric acid, ethanoic acid, carbonic acid, nitric acid, or phosphoric acid, is used to solubilize the source extracellular matrix material. In various illustrative embodiments, the acidic conditions for solubilization can include solubilization at about 0° C. to about 60° C., and incubation periods of about 5 minutes to about 96 hours. In other illustrative embodiments, the concentration of the acid, such as hydrochloric acid, can be from about 0.001 N to about 0.1 N, from about 0.005 N to about 0.1 N, from about 0.01 N to about 0.1 N, from about 0.05 N to about 0.1 N, from about 0.001 N to about 0.05 N, about 0.001 N to about 0.01 N, or from about 0.01 N to about 0.05 M. However, the solubilization can be conducted at any temperature, for any length of time, and at any concentration of acid.

Any of the source extracellular matrix materials described above can be used and the solubilization step can be performed in the presence of an acid or in the presence of an acid and an enzyme. The acid solubilization step results in a solubilized extracellular matrix composition that remains bioactive (i.e., is capable of polymerizing and remodeling tissues in vivo) after lyophilization.

In one illustrative embodiment, the extracellular matrix material is treated with one or more enzymes before, during, or after the acid solubilization step. For enzymes that are inactive at acidic pH, for example, the extracellular matrix material is treated with the enzyme before the acid solubilization step or after the acid solubilization step, but under conditions that are not acidic. Enzymatic digestion of the extracellular matrix material is conducted under conditions that are optimal for the specific enzyme used and under conditions that retain the ability of the solubilized components of the extracellular matrix material to polymerize. The concentration of the enzyme depends on the specific enzyme used, the amount of extracellular matrix material to be digested, the desired time of digestion, and the desired temperature of the reaction. In various illustrative embodiments, about 0.01% to about 0.5% (weight per volume such that 0.01% is equivalent to 0.01 g/100 ml) of enzyme is used. Exemplary enzymes include pepsin, bromelain, cathepsins, chymotrypsin, elastase, papain, plasmin, subtilisin, thrombin, trypsin, matrix metalloproteinases (e.g., stromelysin, elastase), glycosaminoglycan-specific enzymes (e.g., chondroitinase, hyaluronidase, heparinase) and the like, or combinations thereof. The source extracellular matrix material can be treated with one or more enzymes. In illustrative embodiments, the enzyme digestion can be performed at about 2° C. to about 37° C. However, the digestion can be conducted at any temperature, for any length of time (e.g., about 5 minutes to about 96 hours), and at any enzyme concentration.

In illustrative embodiments, the ratio of the extracellular matrix material (hydrated) to total enzyme (weight/weight) ranges from about 25 to about 2500. If an enzyme is used, it should be removed (e.g., by fractionation) or inactivated after the desired incubation period for the digestion so as to not compromise stability of the components in the solubilized extracellular matrix composition. Enzymes, such as pepsin for example, can be inactivated with protease inhibitors, a shift to neutral pH, a drop in temperature below 0° C., or heat inactivation, or a combination of these methods.

In another illustrative embodiment, the source extracellular matrix material can be extracted in addition to being solubilized with hydrochloric acid. Extraction methods for extracellular matrices are known to those skilled in the art and are described in detail in U.S. Pat. No. 6,375,989, incorporated herein by reference. Illustrative extraction excipients include, for example, chaotropic agents such as urea, guanidine, sodium chloride, magnesium chloride, and non-ionic or ionic surfactants.

In one embodiment, the solubilized collagen composition comprises soluble and insoluble components, and at least a portion of the insoluble components of the solubilized collagen composition can be separated from the soluble components. For example, the insoluble components can be separated from the soluble components by centrifugation (e.g., at 12,000 rpm for 20 minutes at 4° C.). In alternative embodiments, other separation techniques known to those skilled in the art, such as filtration, can be used.

In accordance with one illustrative embodiment, the solubilized extracellular matrix composition, prepared with or without the above-described separation step, is fractionated prior to polymerization. In one illustrative aspect, the solubilized extracellular matrix composition can be fractionated by dialysis.

Exemplary molecular weight cut-offs for the dialysis tubing or membrane are from about 3,500 to about 12,000 daltons or about 3,500 to about 5,000 daltons. In one embodiment, the solubilized extracellular matrix composition is dialyzed against an acidic solution having a low ionic strength. For example, the solubilized extracellular matrix composition can be dialyzed against a hydrochloric acid solution, however any other acids can be used, including acetic acid, formic acid, citric acid, lactic acid, sulfuric acid, ethanoic acid, carbonic acid, nitric acid, or phosphoric acid. In another example, the extracellular matrix composition can be dialyzed against water as long as the pH is approximately 6 or below.

In various illustrative embodiments, the fractionation, for example by dialysis, can be performed at about 2° C. to about 37° C. for about 1 hour to about 96 hours. In another illustrative embodiment, the concentration of the acid, such as acetic acid, hydrochloric acid, formic acid, citric acid, lactic acid, sulfuric acid, ethanoic acid, carbonic acid, nitric acid, or phosphoric acid, against which the solubilized extracellular matrix composition is dialyzed, can be from about 0.001 N to about 0.1 N, from about 0.005 N to about 0.1 N, from about 0.01 N to about 0.1 N, from about 0.05 N to about 0.1 N, from about 0.001 N to about 0.05 N, about 0.001 N to about 0.01 N, or from about 0.01 N to about 0.05 N. In one illustrative embodiment, the solubilized extracellular matrix composition is dialyzed against 0.01 N HCl. However, the fractionation can be performed at any temperature, for any length of time, and against any concentration of acid.

In accordance with one embodiment the 3D matrix used for culturing stem cells comprises a lyophilized, solubilized collagen composition that is rehydrated prior to contact with the cells. As discussed above, the term “lyophilized” means that water is removed from the composition, typically by freeze-drying under a vacuum (typically to dryness). In one illustrative aspect, a solubilized extracellular matrix composition is lyophilized after solubilization. In another illustrative aspect, the solubilized extracellular matrix composition is lyophilized after the solubilized portions have been separated from the insoluble portions. In yet another illustrative aspect, the solubilized extracellular matrix composition is lyophilized after a fractionation step but prior to polymerization. In another illustrative embodiment, the polymerized matrix is lyophilized. In one illustrative lyophilization embodiment, the solubilized extracellular matrix composition is first frozen, and then placed under a vacuum. In another lyophilization embodiment, the solubilized extracellular matrix composition is freeze-dried under a vacuum. Any method of lyophilization known to the skilled artisan can be used.

In accordance with one embodiment, the solubilized collagen composition is sterilized before polymerization. In one embodiment the source of the solubilized collagen (e.g., a naturally occurring extracellular matrix, or a lyophilized purified collagen composition) is sterilized prior to the solubilization step. Sterilization of the extracellular matrix material can be performed, for example, as described in U.S. Pat. Nos. 4,902,508 and 6,206,931, incorporated herein by reference. In another embodiment, the solubilized collagen composition is directly sterilized, for example, with peracetic acid. In one embodiment wherein an extracellular matrix is solubilized with an acid and the resulting material is fractionated to isolate a fraction comprising solubilized collagen, sterilization can be carried out either before or after the fractionation step. In another illustrative embodiment, the lyophilized composition itself is sterilized before rehydration, for example using an e-beam sterilization technique. In yet another illustrative embodiment, the polymerized matrix formed from the components of the solubilized collagen matrix composition is sterilized.

In one illustrative embodiment, the solubilized extracellular matrix composition is directly sterilized before the fractionation/separation step, for example, with peracetic acid or with peracetic acid and ethanol (e.g., by the addition of 0.18% peracetic acid and 4.8% ethanol to the solubilized extracellular matrix composition before the separation step). In another embodiment, sterilization can be carried out during the fractionation step. For example, the solubilized extracellular matrix composition can be dialyzed against chloroform, peracetic acid, or a solution of peracetic acid and ethanol to disinfect or sterilize the solubilized extracellular matrix composition. For example, the solubilized extracellular matrix composition can be sterilized by dialysis against a solution of peracetic acid and ethanol (e.g., 0.18% peracetic acid and 4.8% ethanol). The chloroform, peracetic acid, or peracetic acid/ethanol can be removed prior to polymerization of the solubilized collagen composition, for example by dialysis against an acid, such as 0.01 N HCl.

If the solubilized collagen composition is lyophilized, the lyophilized collagen matrix composition can be stored frozen or at room temperature (for example, at about −80° C. to about 25° C.). Storage temperatures are selected to stabilize the components of the solubilized collagen matrix composition. The compositions can be stored for about 1-26 weeks, or longer. In one illustrative embodiment, the storage solvent is hydrochloric acid. As described herein, “storage solvent” means the solvent that the solubilized collagen matrix composition is in prior to and during lyophilization. For example, hydrochloric acid, or other acids, at concentrations of from about 0.001 N to about 0.1 N, from about 0.005 N to about 0.1 N, from about 0.01 N to about 0.1 N, from about 0.05 N to about 0.1 N, from about 0.001 N to about 0.05 N, from about 0.001 N to about 0.01 N, or from about 0.01 N to about 0.05 N can be used as the storage solvent for the lyophilized, solubilized collagen matrix composition. Other acids can be used as the storage solvent including acetic acid, formic acid, citric acid, lactic acid, sulfuric acid, ethanoic acid, carbonic acid, nitric acid, or phosphoric acid, and these acids can be used at any of the above-described concentrations. In one illustrative embodiment, the lyophilizate can be stored (i.e., lyophilized in) an acid, such as acetic acid, at a concentration of from about 0.001 M to about 0.5 M or from about 0.01 M to about 0.5 M. In another embodiment, the lyophilizate can be stored in water with a pH of about 6 or below. In other illustrative embodiments, lyoprotectants, cryoprotectants, lyophilization accelerators, or crystallizing excipients (e.g., ethanol, isopropanol, mannitol, trehalose, maltose, sucrose, tert-butanol, and Tween 20), or combinations thereof, and the like can be present during lyophilization.

In one embodiment, the sterilized, solubilized collagen composition can be dialyzed against 0.01 N HCl, for example, prior to lyophilization to remove the sterilization solution and so that the solubilized extracellular matrix composition is in a 0.01 N HCl solution as a storage solvent. Alternatively, the solubilized extracellular matrix composition can be dialyzed against acetic acid as the storage solvent, for example, prior to lyophilization and can be lyophilized in acetic acid and redissolved in HCl or water.

If the solubilized collagen composition is lyophilized, the resulting lyophilizate can be redissolved in any solution, but may be redissolved in an acidic solution or water. The lyophilizate can be redissolved in, for example, acetic acid, hydrochloric acid, formic acid, citric acid, lactic acid, sulfuric acid, ethanoic acid, carbonic acid, nitric acid, or phosphoric acid, at any of the above-described concentrations, or can be redissolved in water. In one illustrative embodiment the lyophilizate is redissolved in 0.01 N HCl. For use in producing engineered matrices that can be injected in vivo or used for other purposes in vitro, the redissolved lyophilizate can be subjected to varying conditions (e.g., pH, phosphate concentration, temperature, buffer composition, ionic strength, and composition and concentration of solubilized extracellular matrix composition components (dry weight/ml)) that result in polymerization to form 3D matrices for specific tissue graft applications.

Accordingly, in one illustrative embodiment of the method described herein, a solubilized collagen composition is prepared by enzymatically treating the source extracellular matrix material with 0.1% (w/v) pepsin in 0.01 N HCl to initially solubilized the extracellular matrix material, centrifuging the enzymatically treated composition at 12,000 rpm for 20 minutes at 4° C. to remove insoluble components, fractionating the soluble fraction by dialysis against a 0.01 N HCl solution, and then polymerizing the dialyzed fraction.

In another illustrative embodiment, the method does not involve a fractionation step. In this embodiment, the source extracellular matrix material is enzymatically treated with 0.1% (w/v) pepsin in a 0.01 N hydrochloric acid solution to produce a solubilized collagen composition, the solubilized composition is then centrifuged to remove insoluble components, and then the solubilized fraction is polymerized.

In another illustrative embodiment, a solubilized collagen composition is prepared by grinding source vertebrate submucosa into a powder and enzymatically digesting the powderized submucosa with 0.1% (w/v) pepsin and solubilizing in 0.01 N HCl for one to three days at 4° C. Following digestion and solubilization, the solubilized components of the solubilized submucosa composition are separated from the insoluble components by centrifugation at 12,000 rpm at 4° C. for 20 minutes. The supernatant, comprising the soluble components, is recovered and the pellet containing insoluble components is discarded. The supernatant is then fractionated by dialyzing the solubilized submucosa composition against 0.01 N HCl. In one embodiment, the solubilized submucosa composition is dialyzed against several changes of 0.01 N hydrochloric acid at 4° C. using dialysis membranes having a molecular weight cut-off of 3500. Thus, the solubilized submucosa composition is fractionated to remove components having a molecular weight of less than about 3500. Alternatively, dialysis tubing or membranes having a different molecular weight cut-off can be used. The fractionated solubilized submucosa composition is then polymerized to produce the collagen based matrices of the present invention.

In accordance with another illustrative embodiment, a solubilized collagen composition is prepared by grinding vertebrate submucosa into a powder and digesting the powderized submucosa composition with 0.1% (w/v) pepsin and solubilizing in 0.01 N hydrochloric acid for one to three days at 4° C. The solubilized components are then separated from the insoluble components, for example, by centrifugation at 12,000 rpm at 4° C. for 20 minutes. The supernatant, comprising the soluble components, is recovered and the pellet containing insoluble components is discarded. The non-fractionated solubilized submucosa composition is then polymerized.

The present invention encompasses the formation of a solubilized collagen composition from a complex extracellular matrix material without purification of the matrix components. However, the components of the naturally occurring extracellular matrices can be partially purified and the partially purified composition can be used in accordance with the methods described herein to prepare a solubilized collagen composition. Purification methods for extracellular matrix components are known to those skilled in the art and are described in detail in U.S. Pat. No. 6,375,989, incorporated herein by reference. In accordance with one embodiment the solubilized collagen composition includes purified type I collagen or type I and type III collagen as the only protein constituents of the composition.

The solubilized collagen composition can be polymerized under different conditions to produce a collagen based matrix having the desired microstructures and mechanical properties. Polymerization of purified type I collagen solutions at different concentrations of collagen affected fibril density while maintaining a relatively constant fibril diameter. In addition, both fibril length and diameter are affected by altering the pH of the polymerization reaction.

Additional conditions can be varied during the polymerization reaction to provide engineered purified collagen matrices that have the desired properties. In illustrative embodiments, the conditions that can be varied include pH, phosphate concentration, temperature, buffer composition, ionic strength, the extracellular matrix components in the solubilized extracellular matrix composition, and the concentration of solubilized extracellular matrix composition components (dry weight/ml). These conditions result in polymerization of the extracellular matrix components to form engineered extracellular matrices with desired compositional, microstructural, and mechanical characteristics. Illustratively, these compositional, microstructural, and mechanical characteristics can include fibril length, fibril diameter, number of fibril-fibril connections, fibril density, fibril organization, matrix composition, three-dimensional shape or form, viscoelastic, tensile, or compressive behavior, shear (e.g., failure stress, failure strain, and modulus), permeability, swelling, hydration properties (e.g., rate and swelling), and in vivo tissue remodeling and bulking properties, and desired in vitro cell responses. The matrices described herein have desirable biocompatibility, vascularization, remodeling, and bulking properties, among other desirable properties.

In various illustrative embodiments, qualitative and quantitative microstructural characteristics of the engineered matrices can be determined by environmental or cryostage scanning electron microscopy, transmission electron microscopy, confocal microscopy, second harmonic generation multi-photon microscopy. In another embodiment, polymerization kinetics may be determined by spectrophotometry or time-lapse confocal reflection microscopy. In another embodiment, tensile, compressive and viscoelastic properties can be determined by rheometry or uniaxial tensile testing. In another embodiment, a rat subcutaneous injection model can be used to determine remodeling and bulking properties. All of these methods are known in the art or are further described in Examples 5-7 or are described in Roeder et al., J. Biomech. Eng. vol. 124, pp. 214-222 (2002) and in Pizzo et al., J. Appl. Physiol., vol. 98, pp. 1-13 (2004), incorporated herein by reference.

In accordance with one embodiment, the solubilized collagen composition is polymerized at a final total collagen concentration of about 1 to about 40 mg/ml, and in one embodiment about 1 to about 30 mg/ml, in another embodiment about 2 to about 25 mg/ml and in another embodiment about 5 to about 15 mg/ml. In one embodiment the final total collagen is selected from a range of about 0.25 to about 5.0 mg/ml, or in another embodiment the final total collagen concentration is selected from the range of about 0.5 to about 4.0 mg/ml, and in another embodiment the final total collagen concentration is selected from the range of about 1.0 to about 3.0 mg/ml, and in another embodiment the final total collagen concentration is about 0.3, 0.5, 1.0, 2.0 or 3.0 mg/ml. In other embodiments, the components of the solubilized extracellular matrix composition are polymerized at final concentrations (dry weight/ml) of about 0.25 to about 10 mg/ml, about 0.25 to about 20 mg/ml, about 0.25 to about 30 mg/ml, about 0.25 to about 40 mg/ml, about 0.25 to about 50 mg/ml, about 0.25 to about 60 mg/ml, or about 0.25 to about 80 mg/ml.

In various illustrative embodiments, the total collagen comprising the solubilized collagen composition comprises type I and type III collagen, wherein the percent range of the type III collagen and type I collagen is selected from about 17-33% and about 66-83%, respectively, to achieve various collagen type I/III ratios. Examples of percentage ranges of type III collagen and type I collagen, respectively that may be used in the matrices include 17% and 83%; 20% and 80%; 25% and 75%; 30% and 70%; and 33% and 66%, respectively. In various illustrative embodiments, the type I collagen to type III collagen ratio may be in the range of about 6:1 to about 1:1. Examples of the type I collagen to type III collagen ratios that may be used in the matrices include 6:1, 5:1, 4:1, 3:1, 2:1, 1.5:1, and 1:1.

In various illustrative embodiments, at least 3 ug/ml of type I collagen is combined with at least 0.5 ug/ml of type III collagen to obtain a total amount of collagen. Examples of the amount of type I collagen combined with type III collagen, respectively, that may be used in the matrices include 3 ug/ml and 0.5 ug/ml; 1500 ug/ml and 250 ug/ml; 1500 ug/ml and 500 ug/ml; 1500 ug/ml and 750 ug/ml; and 1500 ug/ml and 1500 ug/ml.

In various illustrative embodiments, the conditions for combining type I collagen and type III collagen can be the same as those described above for the method of decreasing stiffness of an extracellular matrix composition.

Illustratively, the matrix compositions produced by the methods described herein can be combined, prior to, during, or after polymerization, with cells, including stem cells or progenitor cells, to further enhance the repair or replacement of diseased or damaged tissues. Examples of progenitor cells include those that give rise to blood, fibroblasts, endothelial cells, epithelial cells, smooth muscle cells, skeletal muscle cells, cardiac muscle cells, multi-potential progenitor cells, pericytes, and osteogenic cells. The population of progenitor cells can be selected based on the cell type of the intended tissue to be repaired. For example, if skin is to be repaired, the population of progenitor cells will give rise to non-keratinized epithelial cells or if cardiac tissue is to be repaired, the progenitor cells can produce cardiac muscle cells. The matrix composition can also be seeded with autogenous cells isolated from the patient to be treated. In an alternative embodiment the cells may be xenogeneic or allogeneic in nature.

In any of the embodiments described above using purified collagen, the purified collagen can be sterilized after purification. In yet other embodiments, the collagen that is purified can be sterilized before or during the purification process. In other embodiments, purified collagen can be sterilized before polymerization or the matrix can be sterilized after polymerization.

It has been reported that the use of progenitor or stem cells to treat damaged tissues (including for example treating myocardial infarction followed by heart failure) has demonstrated early evidence of potential utility. However, recent data, has revealed three key issues that significantly limit successful delivery of reparative cells to tissues. These are 1.) inefficient and inconsistent local retention of cells acutely following injection into tissues [Hou et al., 2005, Circulation, 112:1150-6]; 2.) limited survival of cells over time following injection into tissues [Rehman et al., 2004, Circulation 109: 1292-8]; and 3.) lack of a suitable cellular microenvironment to modulate differentiation into the desired tissue types (e.g., either vascular structures or myocytes in the context of tissue remodeling in response to ischemic insult) [Reinlib and Field, 2000, Circulation 101: E182-E187].

In accordance with one embodiment a novel cell delivery strategy is provided that involves the suspension of cells in a liquid-phase, injectable solubilized collagen composition that polymerizes in situ to form a three-dimensional (3D) matrix. The 3D matrix is designed to both entrap cells and provide them with an “instructive” microenvironment which promotes cell survival and modulates their fate. It is anticipated that the introduction of cells in the presence of a comparatively viscous medium (i.e., the solubilized collagen composition, which will subsequently assemble in situ shortly after post-injection) will enhance the cells local retention. Furthermore, as noted in Examples 12-15, the components of the 3D matrix and their microstructural organization play an important role in determining cell fate with respect to survival, proliferation, and differentiation. Interestingly, recent data shows that a nanofiber microenvironment formed intramyocardially following injection of a peptide (8-16 amino acids long) hydrogel (of which the biological signaling capacity and degradation properties have yet to be elucidated) resulted in formation of a nanofiber microenvironment that promoted endogenous cell recruitment [Davis et al., 2005 Circulation 111:442-50]. Furthermore, co-culture of endothelial cells with cardiomyocytes within the peptide hydrogel in vitro dramatically decreased apoptosis and necrosis of cardiomyocytes [Narmaneva et al., 2004 Circulation 110:962-968].

As reported herein, the biophysical signals provided by a 3D self-assembled collagen microenvironment can be used to direct the proliferation and differentiation capacity of multi-potential, bone marrow-derived stem cells. For example, 3D purified collagen matrices characterized by a relatively high fibril density and stiffness supported an increase in clonal growth and enhanced osteogenesis (bone formation). Collectively, these results demonstrate the ability to engineer injectable, self-assembling 3D purified collagen matrices in which the composition, microstructure, and mechanical properties are defined and systematically varied with discrete outcomes. In general, the biophysical features of the 3D matrix, in addition to cellular signaling modalities consisting of soluble factors and cell-cell interactions, are determinants of cell fate and represent a new target for therapeutic manipulation.

In accordance with one embodiment a method of enhancing the repair of damaged, diseased or congenitally defective tissues is provided. The method comprises the steps of suspending a population of cells within a solubilized collagen composition, inducing the polymerization of the solubilized collagen composition, and injecting the composition into warm blooded species. In one embodiment the method comprises the steps of suspending a population of cells within a first solubilized collagen composition, inducing the polymerization of the first solubilized collagen composition to form a first three dimensional matrix comprising cells, suspending the first three dimensional matrix, or fragments thereof within a second solubilized collagen composition (optionally in the presence of a second population of cells), inducing the polymerization of the second solubilized collagen composition to form a composite tissue graft comprising a first three dimensional matrix entrapped within a second three dimensional matrix. This composite tissue graft can be surgically implanted in the patient or the composition can be injected into the host if the composition is injected prior to the polymerization of the second solubilized collagen composition.

In accordance with one embodiment, the collagen compositions are injected into a mammalian species, including a human for example, and in one embodiment the cells of the matrices represent autologous cells. In an alternative embodiment the cells may be xenogeneic or allogeneic cells. The injected solubilized collagen composition polymerizes in vivo to form a 3D matrix with the population of cells embedded within the collagen matrix. In one embodiment the population of cells comprise stem cells. In one embodiment the soluble collagen composition comprises purified type I collagen, glucose, and calcium chloride. In one embodiment a 3D purified collagen matrix is provided comprising collagen fibrils at a fibril area fraction of about 12% to about 25% (area of fibril to total area) wherein the matrix further comprises exogenously added glucose and CaCl₂. In one embodiment the solubilized collagen composition comprises about 0.05 mg/ml to about 5 mg/ml total purified collagen (either type I alone or a combination of type I and type III collagen) about 1.11 mM to about 277.5 mM glucose and about 0.2 mM to about 4.0 mM CaCl₂. Applicants have discovered that the inclusion of glucose and CaCl₂ within the interstitial fluid of the 3D matrices enhances the survival and functioning of cells seeded within the 3D matrix.

In one embodiment the solubilized collagen composition comprises about 0.1 mg/ml to about 3 mg/ml total purified collagen (either type I alone or a combination of type I and type III collagen) in about 0.05 to about 0.005N HCl (and in one embodiment about 0.01 N HCl), about 0.07M to about 0.28M NaCl (and in one embodiment about 0.137M NaCl), about 1.3 to about 4.5 mM KCl (and in one embodiment about 2.7 mM KCl), about 4.0 to about 16 mM Na₂HPO₄ (and in one embodiment about 8.1 mM Na₂HPO₄), about 0.7 to about 3.0 mM KH₂PO₄ (and in one embodiment about 1.5 mM KH₂PO₄), about 0.25 to about 11.0 mM MgCl₂ (and in one embodiment about 0.5 mM MgCl₂), about 2.8 mM to about 166 mM glucose (and in one embodiment about 5 mM glucose). Polymerization of the solubilized collagen composition is induced by the addition of a neutralizing solution such as NaOH. For example a NaOH solution can be added to a final concentration of 0.01N NaOH. The cells are then added to the composition after the addition of neutralizing solution. In accordance with one embodiment a calcium chloride solution is also added to the solubilized collagen composition. In this embodiment, calcium chloride is added to bring the final concentration of CaCl₂ in the solubilized collagen composition to about 0.4 mM to about 2.0 mM CaCl₂ (and in one embodiment about 0.9 mM CaCl₂). The composition is then allowed to polymerize either in vitro or in vivo to form a 3D matrix comprised of collagen fibrils wherein the cells are embedded within the 3D matrix.

In illustrative embodiments the polymerization reaction is conducted in a buffered solution using any biologically compatible buffer system known to those skilled in the art. For example the buffer may be selected from the group consisting of phosphate buffer saline (PBS), Tris(hydroxymethyl) aminomethane Hydrochloride (Tris-HCl), 3-(N-Morpholino) Propanesulfonic Acid (MOPS), piperazine-n,n′-bis(2-ethanesulfonic acid) (PIPES), [n-(2-Acetamido)]-2-Aminoethanesulfonic Acid (ACES), N-[2-hydroxyethyl]piperazine-N′-[2-ethanesulfonic acid] (HEPES) and 1,3-bis[tris(Hydroxymethyl)methylamino]propane (Bis Tris Propane). In one embodiment the buffer is PBS, Tris or MOPS and in one embodiment the buffer system is PBS, and more particularly 10×PBS. In accordance with one embodiment the 10×PBS buffer at pH 7.4 comprises the following ingredients:

1.37M NaCl

0.027M KCl

0.081M Na₂HPO₄

0.015M KH₂PO₄

5 mM MgCl₂

55.5 mM glucose

To create 10×PBS buffers of different pH, the ratio of Na₂HPO₄ and KH₂PO₄ is varied. Ionic strength may be adjusted as an independent variable by varying the molarity of NaCl only

The polymerization of the solubilized collagen composition is conducted at a pH selected from the range of about 6.0 to about 9.0, and in one embodiment polymerization is conducted at a pH selected from the range of about 5.0 to about 11.0 and in one embodiment about 6.0 to about 9.0, and in one embodiment polymerization is conducted at a pH selected from the range of about 6.5 to about 8.5, in another embodiment polymerization of the solubilized collagen composition is conducted at a pH selected from the range of about 7.0 to about 8.0, and in another embodiment polymerization of the solubilized collagen composition is conducted at a pH selected from the range of about 7.3 to about 7.4.

The ionic strength of the buffered solution is also regulated. In accordance with one embodiment the ionic strength of the solubilized collagen composition is selected from a range of about 0.05 to about 1.5 M, in another embodiment the ionic strength is selected from a range of about 0.10 to about 0.90 M, in another embodiment the ionic strength is selected from a range of about 0.14 to about 0.30 M and in another embodiment the ionic strength is selected from a range of about 0.14 to about 0.17 M.

In still other illustrative embodiments, the polymerization is conducted at temperatures selected from the range of about 0° C. to about 60° C. In other embodiments, polymerization is conducted at temperatures above 20° C., and typically the polymerization is conducted at a temperature selected from the range of about 20° C. to about 40° C., and more typically the temperature is selected from the range of about 30° C. to about 40° C. In one embodiment the polymerization is conducted at about 37° C.

In yet other embodiments, the phosphate concentration is varied. For example, in one embodiment, the phosphate concentration is selected from a range of about 0.005 M to about 0.5 M. In another illustrative embodiment, the phosphate concentration is selected from a range of about 0.01 M to about 0.2 M. In another embodiment, the phosphate concentration is selected from a range of about 0.01 M to about 0.1 M. In another illustrative embodiment, the phosphate concentration is selected from a range of about 0.01 M to about 0.03 M. In other illustrative embodiments, the solubilized collagen composition can be polymerized by, for example, dialysis against a solution under any of the above-described conditions (e.g., dialysis against PBS at pH 7.4), extrusion or co-extrusion of submucosa formulations into a desired buffer, including the buffers described above, or wet-spinning to form strands of extracellular matrix material. In one embodiment the strands can be formed by extrusion of a solubilized collagen composition through a needle and can be air-dried to form threads.

In one embodiment the strands can be formed by extrusion through a needle and can be air-dried to form fibers or threads of various dimensions. The syringe can be adapted with needles or tubing to control the dimensions (e.g., diameter) of the fibers or threads. In one embodiment, the extrusion process involves polymerization of the solubilized extracellular matrix composition followed by extrusion into a bath containing water, a buffer, or an organic solvent (e.g., ethanol).

In another embodiment, the extrusion process involves coextrusion of the solubilized extracellular matrix composition with a polymerization buffer (e.g., the buffer such as Tris or phosphate buffers at various concentrations can be varied to control pH and ionic strength). In yet another embodiment, the extrusion process involves extrusion of the solubilized extracellular matrix composition into a polymerization bath (e.g., the buffer such as Tris or phosphate buffers at various concentrations can be varied to control pH and ionic strength). The bath conditions affect polymerization time and properties of the fibers or threads, such as mechanical integrity of the fibers or threads, fiber dimensions, and the like. In one embodiment the extrusion of a solubilized collagen composition through a needle is used a method to control orientation of polymerized fibrils within the fibers. In one embodiment, the fibers can be air-dried to, create materials that can be crosslinked or woven into three dimensional meshes or mats that can serve as a substrate, or a component of a substrate, for culturing cells. In various illustrative embodiments, engineered extracellular matrices can be polymerized from the solubilized extracellular matrix composition at any step in the above-described methods. For example, the engineered matrices can be polymerized from the solubilized extracellular matrix composition after the solubilization step or after the separation step, the filtration step, or the lyophilization and rehydration steps, if the separation step, the filtration step, and/or the lyophilization and rehydration steps are performed.

In accordance with one embodiment a first solubilized collagen composition is polymerized (optionally in the presence of cells) during an extrusion process to form multiple pieces of a first three dimensional matrix. The pieces of 3D matrix are formed having sufficiently small dimensions that they can be suspended in a second solubilized collagen composition and injected into a host, such as a warm blooded vertebrate species. Alternatively, a first solubilized collagen composition is polymerized (in the presence of cells) to form a monolith structure that is subsequently fractionated into smaller pieces, wherein the fractionated pieces are of sufficiently small dimensions that they can be suspended in a second solubilized collagen composition and injected into a host. These extruded or fragmented pieces of 3D matrix/cells are typically suspended in a second solubilized collagen composition that has a different composition than the first solubilized collagen composition (e.g., has a different concentration of total collagen or differs in collagen type). The second solubilized collagen composition is then polymerized (either in vitro or in vivo) to entrap the first extruded or fragmented pieces of 3D matrix/cells within a second 3D matrix formed from the second solubilized collagen composition. The second solubilized collagen composition may optionally include cells. In one embodiment the cells present in either the first solubilized collagen composition, or the second solubilized collagen composition, or both are stem cells. In this manner a composite tissue graft is formed comprising a first 3D matrix (optionally containing a population of cell therein) suspended within a second 3D matrix, wherein the second 3D matrix optionally contains a population of cells entrapped within the second 3D matrix.

The engineered matrices can be combined, prior to, during, or after polymerization, with nutrients, including minerals, amino acids, pharmaceutical agents, sugars, peptides, proteins, vitamins (such as ascorbic acid), or glycoproteins that facilitate cellular proliferation, such as laminin and fibronectin, or growth factors such as epidermal growth factor, platelet-derived growth factor, transforming growth factor beta, or fibroblast growth factor, and glucocorticoids such as dexamethasone. In other illustrative embodiments, fibrillogenesis modulators, such as alcohols, glycerol, glucose, or polyhydroxylated compounds can be added prior to or during polymerization. In accordance with one embodiment, cells can be added to the solubilized extracellular matrix composition as the last step prior to the polymerization or after polymerization of the matrix. In another illustrative embodiment, particulate extracellular matrix compositions can be added to the solubilized extracellular matrix composition and can enhance in vivo bulking capacity. In other illustrative embodiments, cross-linking agents, such as carbodiimides, aldehydes, lysl-oxidase, N-hydroxysuccinimide esters, imidoesters, hydrazides, and maleimides, and the like can be added before, during, or after polymerization.

Hyaluronic acid (HA) is a glycosaminoglycan found naturally within the extracellular matrix. This mucopolysaccharide is made up of a repetitive sequence of two modified simple sugars, glucuronic acid and N-acetyl glucosamine. HA molecules are negatively charged and typically high in molecular weight (long in size). The size and charged nature of this molecule allow it to bind water to produce a high viscosity gel. When hyaluronic acid is added to soluble collagen compositions and the solubilized collagen compositions are allowed to polymerize, it appears that only subtle changes occur to the fibrillar microstructure of the resultant 3D matrix. On the other hand, increasing the hyaluronic acid content significantly affects the viscous fluid phase of the extracellular matrix, providing it with distinct mechanical behavior. Furthermore, the addition of hyaluronic acid to engineered matrices was found to modulate the manner by which cells remodel and contract the matrix. Accordingly, HA content represents a further variable of the present engineered 3D matrices that can be manipulated to provide an optimal microenvironment for cells cultured within the matrices.

In any of the embodiments described in this application, the solubilized collagen composition (i.e., purified collagen or extracellular matrix components) can be polymerized at final concentrations of collagen (dry weight/ml) of about 5 to about 10 mg/ml, about 5 to about 30 mg/ml, about 5 to about 50 mg/ml, about 5 to about 100 mg/ml, about 20 to about 50 mg/ml, about 20 to about 60 mg/ml, or about 20 to about 100 mg/ml. Illustratively, the three-dimensional matrices may contain fibrils with specific characteristics, including, but not limited to, a fibril area fraction (defined as the percent area of the total area occupied by fibrils in a cross-sectional surface of the matrix; i.e., fibril density) of about 7% to about 26%, about 20% to about 30%, about 20% to about 50%, about 20% to about 70%, about 20% to about 100%, about 30% to about 50%, about 30% to about 70%, or about 30% to about 100%. In further illustrative embodiments, the three-dimensional matrices have an elastic or linear modulus (defined by the slope of the linear region of the stress-strain curve obtained using conventional mechanical testing protocols; i.e., stiffness) of about 0.5 kPa to about 40 kPa, about 30 kPa to 100 kPa, about 30 kPa to about 1000 kPa, about 30 kPa to about 10000 kPa, about 30 kPa to about 70000 kPa, about 100 kPa to 1000 kPa, about 100 kPa to about 10000 kPa, or about 100 kPa to about 70000 kPa.

In accordance with one embodiment the 3D matrices of the present invention can be used as cell culture substrates that more accurately mimic the substrates that various cells contact in vivo. Accordingly, collagenous based matrices can be designed for specific cell types to mimic their native environment. In this manner cell, including stem cells or progenitor cells, can be cultured in vitro without altering the fundamental cell behavior (e.g., cell proliferation, growth, maturation, differentiation, migration, adhesion, gene expression, apoptosis and other cell behaviors) of the cells. In another embodiment, the engineered purified collagen based matrices of the present invention can be used to expand or differentiate a cell population, such a stem cell population (including pluripotent or unipotent cells), primary cells, progenitor cells or other eukaryotic cells by seeding the cells on, or within, the collagen based matrix and culturing the cells in vitro for a predetermined length of time under conditions conducive for that cell type's proliferation (i.e., appropriate nutrients, temperature, pH, etc.). In accordance with one embodiment cells are added to the solubilized collagen composition as the last step prior to the polymerization of the solubilized collagen composition. The engineered purified collagen based matrices of the present invention can be combined with nutrients, including minerals, pharmaceutical agents, amino acids, sugars, peptides, proteins, vitamins (such as ascorbic acid), or glycoproteins that facilitate cellular proliferation, such as laminin and fibronectin and growth factors such as epidermal growth factor, platelet-derived growth factor, transforming growth factor beta, or fibroblast growth factor, and glucocorticoids such as dexamethasone.

In one example of an embodiment comprising a collagen based matrix seeded with living cells, a sterilized engineered purified collagen based matrix may be seeded with living cells and packaged in an appropriate medium for the cell type used. For example, a cell culture medium comprising Dulbecco's Modified Eagles Medium (DMEM) can be used with standard additives such as non-essential amino acids, glucose, ascorbic acid, sodium pyruvate, fungicides, antibiotics, etc., in concentrations deemed appropriate for cell type, shipping conditions, etc.

The cell seeded engineered purified collagen based matrices of the present invention can be used simply for culturing cells in vitro, or the composition can be implanted or injected as a tissue graft construct to enhance the repair of damaged or diseased tissue. In one embodiment an improved tissue graft construct is provided wherein the construct comprises an 3D purified collagen based matrix and a population of cells. The 3D purified collagen based matrix is formed from a solubilized collagen composition wherein the solubilized composition is formed by contacting a source of purified collagen with an acid selected from the group consisting of hydrochloric acid, acetic acid, formic acid, sulfuric acid, ethanoic acid, carbonic acid, nitric acid, or phosphoric acid. The solubilized collagen composition is then polymerized as described above to form the 3D purified collagen based matrix.

Cells, and in one embodiment stem cells, are combined with the collagen based matrix at a low density and can be either added to the solubilized collagen composition prior to polymerization, or after formation of the collagen based matrix. In accordance with one embodiment cells are seeded within the 3D matrix at a final concentration selected from the range of about 10 to about 10⁸ cells/milliliter, and in one embodiment at a final concentration of 10³ to about 10⁵ cells/milliliter. This initial seeded population of cells can be expanded by incubating the composition under conditions suitable for replication of the seeded cells. In accordance with one embodiment cells are initially seeded on or within the 3D matrices at a minimal cell density that will allow for cell viability and replication (i.e., the minimal functionality density). This minimal functionality density can be easily established for the particular cell type to be cultured and for the specific culture conditions utilized.

In accordance with one embodiment the stem cells or progenitor cells are seeded within the collagen based matrix at a cell density substantially higher than the minimal functionality density but at a relative low density compared to standard cell culture techniques. In one embodiment the cells comprise stem cells, wherein the cells are seeded at a density within 3 orders of magnitude of the minimal functionality density, in another embodiment stem cells are seeded at a density within 2 orders of magnitude of the minimal functionality density, and in another embodiment the stem cells are seeded at a density within an order of magnitude of the minimal functionality density. In one embodiment stem cells are seeded at a density of less than 5×10⁴ cells/ml, in another embodiment stem cells are seeded at a density of less than 1×10⁴ cells/ml, in another embodiment stem cells are seeded at a density selected from a range of about 1×10² to about 5×10³

Accordingly, cell seeded 3D purified collagen based matrices of the present invention comprise a population of cells that consists of, or are the progeny of, eukaryotic stem cells initially added to the composition at a low density. In one embodiment a tissue graft construct is prepared comprising the 3D purified collagen based matrices of the present invention that have been seeded with a low density of cells, wherein the cells are cultured within the matrix to expand and/or differentiate the seeded population of cells prior to implantation of the graft construct in a host. In one embodiment the cells, and more particularly stem cells, are initially seeded within the 3D purified collagen matrix at a final concentration of less than 10⁵ cells per milliliter.

For most cells, cell survival during in vitro culture is known to decrease as the concentration/density at which the cells are initially seeded onto a substrate. Applicants have discovered that using an engineered purified collagen based matrix and seeding stem cells at very low densities, clonal populations of stem cells can be isolated in a substantially pure form. Typically the isolation of non-embryoic stem cells results in the isolation of cells that may differentiate along different cell lineage pathways. In accordance with one embodiment of the present invention culturing conditions can be selected wherein a decreased seeding density of viable pluripotent or multipotent stem cells within an engineered purified collagen based matrix leads to clonal growth of cells representing a single cell lineage. Such cells can be isolated and transferred to a second engineered purified collagen based matrix and conditions can be altered to enhance the proliferation of the isolated clonal population of cells. Estimates of optimal cell densities for clonal growth range from about 10 cells/ml to about 10³ cells/ml and depend upon the specific seeding efficiencies.

In accordance with one embodiment a kit is provided for preparing 3D matrices that have been optimized for a particular cell that is to be seeded within the formed 3D matrix. The kit is provided with purified individual components that can be combined to form a solubilized collagen composition that upon polymerization forms a 3D matrix comprised of collagen fibrils that presents an optimal microenvironment for a population of cells. Typically the population of cells represent cells provided separately from the kit, but in one embodiment the cells may also constitute a component of the kit. In one embodiment the cells are mammalian cells, including human cells, and in a further embodiment the cells are stem or progenitor cells. In accordance with one embodiment a kit is provided comprising a solubilized collagen composition and a polymerization agent. In a further embodiment the solubilized collagen composition comprises purified type I collagen as the sole collagen component. In another embodiment the solubilized collagen composition comprises purified type I collagen and type III collagen as the sole collagen components.

In one embodiment the kit comprises separate vessels, each containing one of the following components: purified type I collagen, a phosphate buffer solution, a glucose solution, a calcium chloride solution and a basic neutralizing solution. In one embodiment the purified type I collagen of the kit is provided in a lyophilized form and the kit is further provided with a solution of HCl (or other dilute acid including for example, acetic acid, formic acid, lactic acid, citric acid, sulfuric acid, ethanoic acid, carbonic acid, nitric acid, or phosphoric acid) for resuspending the lyophilized collagen. In one embodiment the kit is provided with a solution comprising a solubilized collagen composition, and in a further embodiment the solubilized collagen composition comprises a solubilized extracellular matrix composition. In one embodiment the kit comprises a phosphate buffer solution, a glucose solution, a calcium chloride solution, and acid solution, a basic neutralizing solution, a vessel comprising purified type I collagen, and a vessel comprising purified type III collagen. In one embodiment the polymerization composition comprises a phosphate buffer that has a pH of about 7.2 to about 7.6 and the acid solution is an HCl solution comprising about 0.05N to about 0.005N HCl, and in one embodiment the acid solution is about 0.01N HCl. In one embodiment the glucose solution has a concentration selected from the range of about 0.2% to about 5% w/v glucose, or about 0.5% to about 3% w/v glucose, and in one embodiment the glucose solution is about 1% w/v glucose. In one embodiment the CaCl₂ solution has a concentration selected from the range of about 2 mM to about 40.0 mM CaCl₂ or about 0.2 mM to about 4.0 mM CaCl₂, or about 0.2 to about 2 mM CaCl₂. In one embodiment the kit is provided with a 10×PBS buffer having a pH of about pH 7.4, and comprising about 1.37M NaCl, about 0.027M KCl, about 0.081M Na₂HPO₄, about 0.015M KH₂PO₄, about 5 mM MgCl₂ and about 1% w/v glucose.

The kit can further be provided with instructional materials describing methods for mixing the kit reagents to prepare 3D matrices. In particular, the instructions materials provide information regarding the final concentrations and relative proportions of the matrix components that give optimal microenvironmental conditions including fibril microstructure and mechanical properties for a particular cell type or for a particular desired result (i.e., clonal expansion of cells, differentiation, etc.).

The following examples illustrate specific embodiments in further detail. These examples are provided for illustrative purposes only and should not be construed as limiting the invention or the inventive concept in any way.

### Example 1

### Preparation of Lyophilized, Bioactive ECM Compositions from Fractionated Submucosa Hydrolysates

Small intestinal submucosa is harvested and prepared from freshly euthanized pigs as previously disclosed in U.S. Pat. Nos. 4,956,178. Intestinal submucosa is powderized under liquid nitrogen and stored at −80° C. prior to use. Digestion and solubilization of the material is performed by adding 5 grams of powdered tissue to each 100 ml of solution containing 0.1% (w/v) pepsin in 0.01 N hydrochloric acid and incubating for 72 hours at 4° C. Following the incubation period, the resulting solubilized composition is centrifuged at 12,000 rpm for 20 minutes at 4° C. and the insoluble pellet is discarded. The supernatant is dialyzed against at least ten changes of 0.01 N hydrochloric acid at 4° C. (MWCO 3500) over a period of at least four days. The solubilized fractionated composition is then sterilized by dialyzing against 0.18% peracetic acid/4.8% ethyl alcohol for about two hours. Dialysis of the composition is continued for at least two more hours, with additional changes of sterile 0.01 N hydrochloric acid per day, to eliminate the peracetic acid. The contents of the dialysis bags are then lyophilized to dryness and stored.

### Example 2

### Preparation of Lyophilized, Bioactive ECM Compositions from Non-Fractionated Submucosa Hydrolysates

Small intestinal submucosa was harvested and prepared from freshly euthanized pigs as previously disclosed in U.S. Pat. Nos. 4,956,178. Intestinal submucosa was powderized under liquid nitrogen and stored at −80° C. prior to use. Partial digestion of the material was performed by adding 5 g powdered tissue to each 100 ml solution containing 0.1% (w/v) pepsin in 0.01M hydrochloric acid and digesting for 72 hours at 4° C. Following partial digestion, the suspension was centrifuged at 12,000 rpm for 20 minutes at 4° C. and the insoluble pellet discarded.

The supernatant was lyophilized to dryness.

### Example 3

### Preparation of Reconstituted, Bioactive ECM Compositions

Immediately prior to use, lyophilized material from Example 2, consisting of a mixture of extracellular matrix components, was reconstituted in 0.01 N HCl. To polymerize the soluble extracellular matrix components into a

Three-dimensional matrix, reconstituted extracellular matrix solutions were diluted and brought to a particular pH, ionic strength, and phosphate concentration by the addition of a phosphate buffer and concentrated HCl and NaOH solutions. Polymerization of neutralized solutions was then induced by raising the temperature from 4° C. to 37° C. Various polymerization buffers (including, e.g., phosphate buffers) were used and the pH of the polymerization reaction was controlled by varying the ratios of mono- and dibasic phosphate salts. Ionic strength was varied based on sodium chloride concentration.

Type I collagen prepared from calf skin was obtained from Sigma-Aldrich Corporation, St. Louis, Mo., and dissolved in and dialyzed extensively against 0.01 M hydrochloric acid (HCl) to achieve desired concentrations. Interstitial ECM was prepared from porcine small intestinal submucosa (SIS). SIS was powdered under liquid nitrogen and the powder stirred (5% w/v) into 0.01 N hydrochloric acid containing 0.1% (w/v) pepsin for 72 h at 4° C. The suspension was centrifuged at 12,000×g for 20 min at 4° C. to remove undissolved tissue particulate and lyophilized to dryness. Immediately prior to experimental use, the lyophilized material was redissolved in 0.01 N HCl to achieve desired collagen concentrations. To polymerize the soluble collagen or interstitial ECM components into a 3D matrix, each solution was diluted and brought to the specified pH, ionic strength, and phosphate concentration by the addition of an polymerization buffer and concentrated HCl and NaOH solutions. Polymerization of neutralized solutions was induced by raising the temperature from 4° C. to 37° C. Various polymerization compositions were used to make final solutions with the properties shown in Table 2.

TABLE 2
Table 2: Engineered ECMs representing purified type I collagen
or a complex mixture of interstitial ECM components (SIS)
were prepared at varied pH (series 1), ionic strength (series
2), and phosphate concentration (series 3).

| Collagen formulations | | | | SIS formulations | | | |
|---|---|---|---|---|---|---|---|
| pH | I | [Pᵢ] | [C] | pH | I | [Pᵢ] | [C] |

Series 1

| | | | | | | | |
|---|---|---|---|---|---|---|---|
| 6.5 | 0.16 | 0.01 | 1 mg/ml | 6.5 | 0.16 | 0.01 | 1 mg/ml |
| 7.0 | 0.16 | 0.01 | 1 mg/ml | 7.0 | 0.16 | 0.01 | 1 mg/ml |
| 7.4 | 0.17 | 0.01 | 1 mg/ml | 7.4 | 0.17 | 0.01 | 1 mg/ml |
| 8.0 | 0.17 | 0.01 | 1 mg/ml | 8.0 | 0.17 | 0.01 | 1 mg/ml |
| 8.5 | 0.17 | 0.01 | 1 mg/ml | 8.5 | 0.17 | 0.01 | 1 mg/ml |
| 9.0 | 0.17 | 0.01 | 1 mg/ml | 9.0 | 0.17 | 0.01 | 1 mg/ml |

Series 2

| | | | | | | | |
|---|---|---|---|---|---|---|---|
| 7.4 | 0.06 | 0.02 | 1 mg/ml | 7.4 | 0.06 | 0.02 | 1 mg/ml |
| 7.4 | 0.10 | 0.02 | 1 mg/ml | 7.4 | 0.30 | 0.02 | 1 mg/ml |
| 7.4 | 0.15 | 0.02 | 1 mg/ml | 7.4 | 0.60 | 0.02 | 1 mg/ml |
| 7.4 | 0.20 | 0.02 | 1 mg/ml | 7.4 | 0.90 | 0.02 | 1 mg/ml |
| 7.4 | 0.25 | 0.02 | 1 mg/ml | 7.4 | 1.20 | 0.02 | 1 mg/ml |
| | | | | 7.4 | 1.50 | 0.02 | 1 mg/ml |

Series 3

| | | | | | | | |
|---|---|---|---|---|---|---|---|
| 7.4 | 0.15 | 0.00 | 1 mg/ml | 7.4 | 0.3 | 0.00 | 1 mg/ml |
| 7.4 | 0.15 | 0.01 | 1 mg/ml | 7.4 | 0.3 | 0.02 | 1 mg/ml |
| 7.4 | 0.15 | 0.02 | 1 mg/ml | 7.4 | 0.3 | 0.04 | 1 mg/ml |
| 7.4 | 0.15 | 0.03 | 1 mg/ml | 7.4 | 0.3 | 0.06 | 1 mg/ml |
| 7.4 | 0.15 | 0.04 | 1 mg/ml | 7.4 | 0.3 | 0.08 | 1 mg/ml |
| 7.4 | 0.15 | 0.05 | 1 mg/ml | 7.4 | 0.3 | 0.11 | 1 mg/ml |

[C] represents collagen concentration in mg/ml, [Pᵢ] represents phosphate concentration in M, and I represents ionic strength in M.

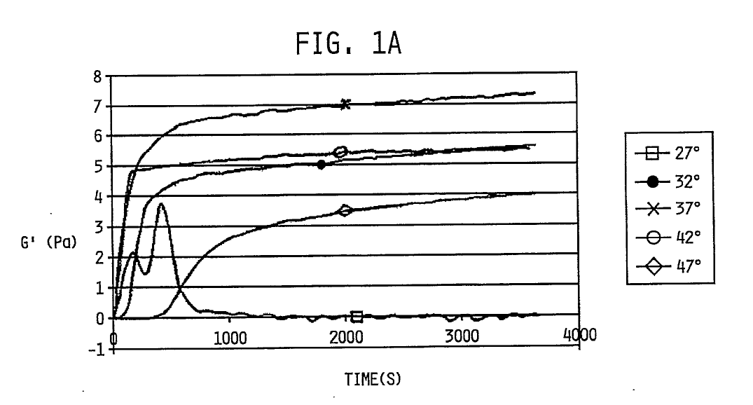

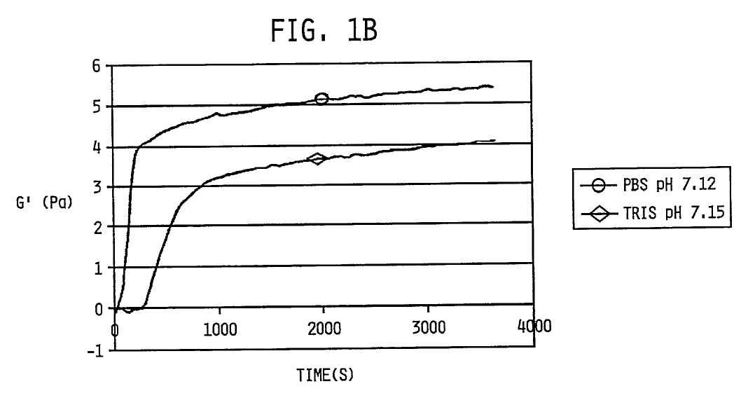

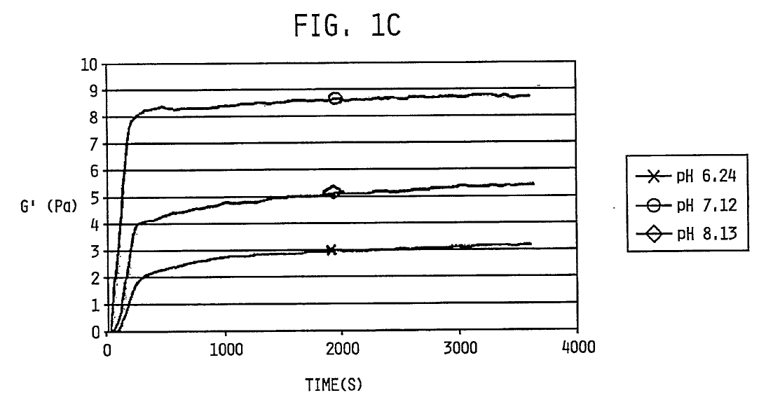

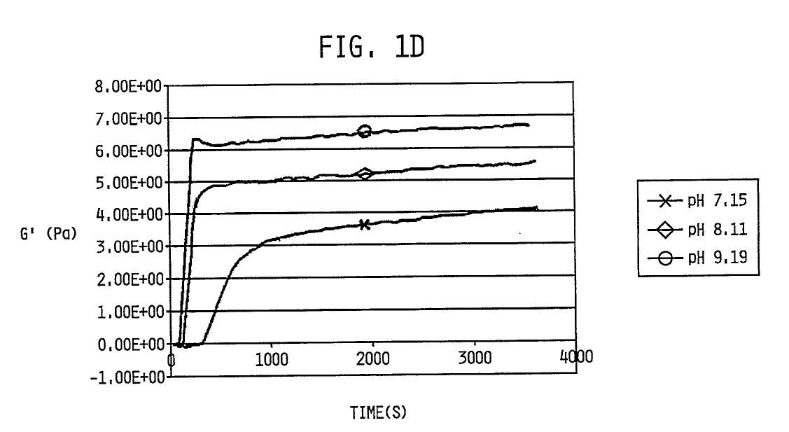

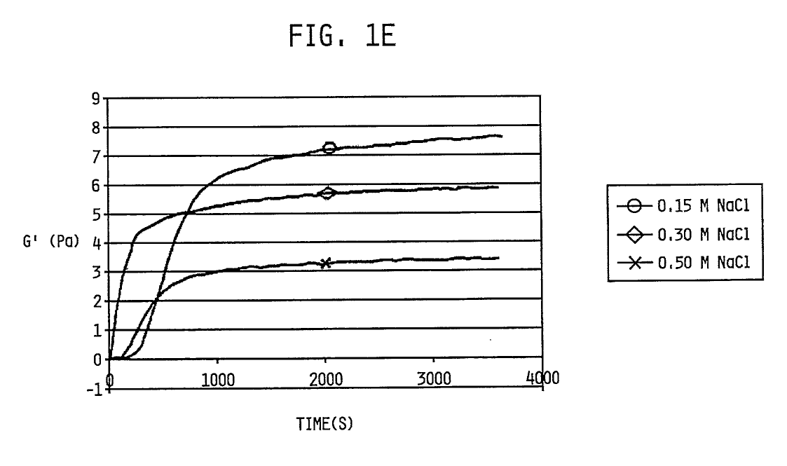

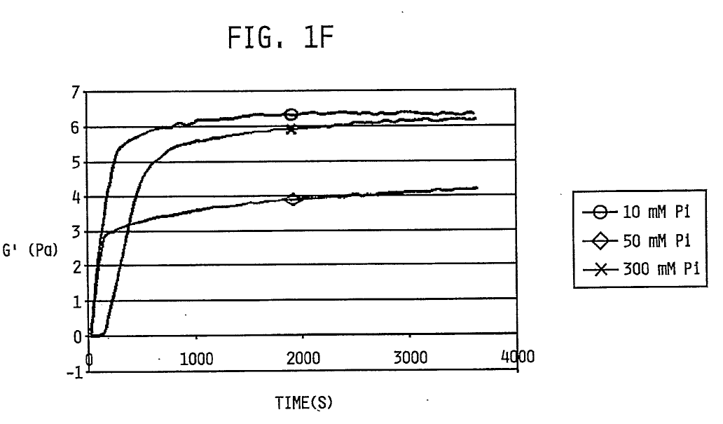

Representative data showing the results of varying the polymerization temperature, buffer system, pH (using either a phosphate or tris buffer), ionic strength, phosphate concentration or concentration of ECM material, on stiffness (elastic modulus) of the formed 3D matrix is presented in FIGS. 1A-1G. In summary, as the polymerization temperature is increased from 4° C. up to 37° C., the polymerization rate and the stiffness of the formed 3D matrix increases. The effect of a temperature gradient profile on the microstructural composition of the 3D matrix was also investigated. Polymerizing the matrix using a temperature ramp from about 4° C. to 37° C. over 30 minutes was compared to matrices formed using a step increase in temperature to 37° C. and incubated at that temperature for 30 minutes. The data revealed that fibrils formed using a temperature ramp are longer in length and have decreased fibril density compared to matrices formed using a single step increase in temperature. As the pH of the polymerizing composition is increased, from about 7.0 up to about pH 9.2, the polymerization rate and the stiffness of the formed 3D matrix increases. Buffer selection was found to play a role in determining the mechanical properties of the 3D matrix, and more particularly tris based buffers reduced stiffness more than phosphate based buffers. Regarding ionic strength, peak stiffness coincides with maximum polymerization time at an ionic strength of about 0.3 M. As phosphate concentration is increased, stiffness decreases, however the concentration of phosphate in a 1×PBS solution does not have a substantial effect on stiffness. As collagen content is increased the stiffness of the matrix is increased.

### Example 4

### Three-Dimensional Imaging of Engineered ECM's by Confocal Reflection Microscopy

Solutions of type I collagen or interstitial ECM components were polymerized in a Lab-Tek chambered coverglass and imaged using a BioRad Radiance 2100 MP Rainbow confocal/multiphoton microscope using a 60×1.4 NA oil immersion lens. Optical settings were established and optimized for matrices after polymerization was complete. Samples were illuminated with 498 nm laser light and the reflected light detected with a photomultiplier tube (PMT) using a blue reflection filter. A z step of 0.2 μm was used to optically section the samples. Because the resolution of the z axis is less than that of the x-y plane, the sampling along the z axis may be different from that of the x-y. Images were collected in the range of 10-25 μm from the upper surface of the coverglass.

### Example 5

### Quantification of Fibril Properties from Three Dimensional Images

Quantification of the fibril diameter distribution within engineered extracellular matrices was conducted based on two- and three-dimensional image sets obtained via electron and confocal microscopy techniques using methods described within Brightman et al., Biopolymers 54:222-234, 2000. More recently, a Matlab program with a graphical user interface was written for measurement of fibril diameters from these images. For three-dimensional confocal images, depth attenuation was corrected by normalizing against a fitted logarithmic curve, after which images were binarized into white and black pixels using a threshold value. Three rectangles were outlined in the x-y plane across each fibril, with one axis aligned with the fibril. Average fibril diameter in each rectangle was calculated as the total white area divided by the rectangle's length. The average diameter of each fibril was taken to be the average of the three measurements, and the average diameter in a given matrix was calculated as an average of all measurements.

Length of fibril per volume was estimated by dividing the total white volume of an image by the average cross-sectional area of fibrils in that image. Due to distortion in the z-plane, the fibril cross-sections in the image could not be assumed circular and calculated from diameter. Rather, the average cross-sectional area was found by expanding the rectangles described above into three-dimensional boxes. The cross-sectional area of a fibril in was found by dividing the total white volume contained in the box by the length of the box's axis aligned with the fibril.

A Matlab program has also been developed to determine fibril density from two- and three-dimensional images. This method involves thresholding and binarizing the image data to discriminate fibrils from the background. The surface area or volume representing fibrils is then quantified and normalized to the surface area or volume of the image.

### Example 6

### Spectrophotometry of Extracellular Matrix Polymerization

The time-course of polymerization was monitored in a Lambda 35 UV-VIS spectrophotometer (Perkin-Elmer) equipped with a temperature-controlled, 8-position cell changer as described previously by Brightman et al., 2000.

### Example 7

### Rheometric Measurements of Extracellular Matrices

Mechanical properties of the matrices were measured using a TA Instruments AR-2000 rheometer. Neutralized collagen or SIS was placed on the peltier temperature-controlled lower plate at 6° C., and the 40-mm parallel-plate geometry was lowered to a 1-mm gap. The temperature was then raised to 37° C. as oscillation measurements were made every 30 seconds at 1 Hz and 5% strain. After polymerization was complete, an oscillation frequency sweep was made at 5% strain, from 0.1 to 3 Hz. A shear creep test was then conducted with a shear stress of 1 Pa for 1000 seconds.

### Example 8

### Preparation of Reconstituted Bioactive Extracellular Matrices

Small intestinal submucosa was harvested and prepared from freshly euthanized pigs as previously disclosed in U.S. Pat. No. 4,956,178. Intestinal submucosa was powderized under liquid nitrogen and stored at −80° C. prior to use. Digestion and solubilization of the material was performed by adding 5 grams of powdered tissue to each 100 ml of solution containing 0.1% pepsin in 0.01 N hydrochloric acid and incubating with stirring for 72 hours at 4° C. Following the incubation period, the solubilized composition was centrifuged at 12,000 rpm for 20 minutes at 4° C. and the insoluble pellet was discarded. The supernatant was dialyzed extensively against 0.01 N HCl at 4° C. in dialysis tubing with a 3500 MWCO (Spectrum Medical Industries). Polymerization of the solubilized extracellular matrix composition was achieved by dialysis against PBS, pH 7.4, at 4° C. for about 48 hours. The polymerized construct was then dialyzed against several changes of water at room temperature and was then lyophilized to dryness.

The polymerized construct had significant mechanical integrity and, upon rehydration, had tissue-like consistency and properties. In one assay, glycerol was added prior to polymerization by dialysis and matrices with increased mechanical integrity and increased fibril length resulted.

### Example 9

### Preparation of Extracellular Matrix Threads

Small intestinal submucosa was harvested and prepared from freshly euthanized pigs as previously disclosed in U.S. Pat. No. 4,956,178. Intestinal submucosa was powderized under liquid nitrogen and stored at −80° C. prior to use. Digestion and solubilization of the material was performed by adding 5 grams of powdered tissue to each 100 ml of solution containing 0.1% (w/v) pepsin in 0.01 N hydrochloric acid and incubating for 72 hours at 4° C. Following the incubation period, the solubilized composition was centrifuged at 12,000 rpm for 20 minutes at 4° C. and the insoluble pellet was discarded.

The solubilized extracellular matrix composition (at 4° C.) was placed in a syringe with a needle and was slowly injected into a PBS solution at 40° C. The solubilized extracellular matrix composition immediately formed a filament with the diameter of the needle. If a blunt-tipped needle is used, straight filaments can be formed while coiled filaments can be formed with a bevel-tipped needle. Such filaments can be used as resorbable sutures.

### Example 10

### Lyophilization and Reconstitution of Solubilized Extracellular Matrix Compositions

Frozen small intestinal submucosa powder that had been prepared by cryogenic milling was centrifuged at 3000×g for 15 minutes and the excess fluid was decanted. The powder (5% weight/volume) was digested and solubilized in 0.01 N HCl containing 0.1% weight/volume pepsin for approximately 72 hours at 4° C. The solubilized extracellular matrix composition was then centrifuged at 16,000×g for 30 minutes at 4° C. to remove the insoluble material. Aliquots of the solubilized extracellular matrix composition were created and hydrochloric acid (12.1 N) was added to create a range of concentrations from 0.01 to 0.5 N HCl.

Portions of the solubilized extracellular matrix composition were dialyzed (MWCO 3500) extensively against water and 0.01 M acetic acid to determine the effects of these media on the lyophilization product. Aliquots of the solubilized extracellular matrix composition in 0.01 M acetic acid were created and glacial acetic acid (17.4 M) was added to create a range of concentrations from 0.01 to 0.5 M acetic acid. The solubilized extracellular matrix compositions were frozen using a dry ice/ethanol bath and lyophilized to dryness. The lyophilized preparations were observed, weighed, and dissolved at 5 mg/ml in either 0.01 N HCl or water. The dissolution and polymerization properties were then evaluated. The results are shown in Tables 2-6.

TABLE 3
Gross appearance of solubilized extracellular
matrix compositions following lyophilization
at various hydrochloric acid concentrations.

| [HCl] (N) | Appearance |
|---|---|
| 0.01 | Light, fluffy, homogenous, foam-like sheet; white to off-white |
| | in color; pliable |
| 0.05 | Slightly wrinkled and contracted, some inhomogeneities in |
| | appearance noted, slight brown tint, pliable to slightly |
| | friable in consistency |
| 0.10 | Wrinkled, collapsed in appearance; inhomogeneities noted, |
| | some regional “melting” noted; significant brown |
| | tint; friable |
| 0.25 | Wrinkled, collapsed in appearance; increased inhomogeneities |
| | noted, increased areas of regional “melting” noted; significant |
| | brown tint; friable |
| 0.50 | Significant collapse and shrinkage of specimen, dark brown |
| | coloration throughout; dark brown in color; friable |

TABLE 4
Dissolution properties of solubilized extracellular
matrix compositions following lyophilization at
various hydrochloric acid concentrations.

| [HCl] (N) | | |
|---|---|---|
| Reconstitution | Reconstitution Properties | |
| Medium | H₂O | 0.01 N HCl |
| 0.01 | Completely dissolved in | Completely dissolved in |
| | 20-30 minutes, pH 4 | 20-30 minutes, pH 2 |
| 0.05 | Majority dissolved in 2 | Majority dissolved in |
| | hours; slight particulate | 40 minutes; very slight |
| | noted, pH 3-4 | particulate noted, pH 2 |
| 0.1 | Incomplete dissolution | Incomplete dissolution |
| 0.25 | Incomplete dissolution | Incomplete dissolution |
| 0.50 | Incomplete dissolution | Incomplete dissolution |

TABLE 5
Polymerization properties of solubilized extracellular
matrix compositions following lyophilization at
various hydrochloric acid concentrations.

| [HCl] (N) | | |
|---|---|---|
| Reconstitution | Polymerization Properties | |
| Medium | H₂O | 0.01 N HCl |
| 0.01 | Polymerized within 20-30 | Polymerized within |
| | minutes | 10-20 minutes |
| 0.05 | Weak polymerization noted | Polymerized within |
| | at 45 minutes; significant | 20-30 minutes |
| | lag time in polymerization | |
| 0.1 | *No Polymerization | *No Polymerization |
| 0.25 | *No Polymerization | *No Polymerization |
| 0.50 | *No Polymerization | *No Polymerization |

TABLE 6
Dissolution properties of solubilized extracellular matrix compositions
following lyophilization at various acetic acid concentrations.

| [Acetic | | |
|---|---|---|
| Acid] | Reconstitution Properties | |
| (M) | Reconstitution in H₂O | Reconstitution in 0.01 N HCl |
| 0.01 | Completely dissolved | Completely dissolved |
| | in 90 minutes, pH 5 | in 90 minutes, pH 1-2 |
| 0.05 | Near complete dissolution | Completely dissolved |
| | after 90 minutes; small | in 90 minutes, pH 1-2 |
| | particulate remained, | |
| | pH 5 | |
| 0.1 | Completely dissolved | Near complete dissolution |
| | in 90 minutes, pH 5 | in 90 minutes; small |
| | | particulate, pH 1-2 |
| 0.25 | Completely dissolved | Completely dissolved |
| | in 90 minutes, pH 5 | in 90 minutes, pH 1-2 |
| 0.50 | Near complete dissolution | Completely dissolved |
| | after 90 minutes; small | in 90 minutes, pH 1-2 |
| | particulate remained, | |
| | pH 5 | |

TABLE 7
Polymerization properties of solubilized extracellular
matrix compositions following lyophilization at
various acetic acid concentrations.

| [Acetic Acid] | | |
|---|---|---|
| (M) | | |
| Reconstitution | Polymerization Properties | |
| Medium | H₂O | 0.01 N HCl |
| 0.01 | Polymerized within 5-10 | Polymerized within 5-10 |
| | minutes | minutes |
| 0.05 | Polymerized within 5-10 | Polymerized within 5-10 |
| | minutes | minutes |
| 0.1 | Polymerized within 5-10 | Polymerized within 5-10 |
| | minutes | minutes |
| 0.25 | Polymerized within 5-10 | Polymerized within 5-10 |
| | minutes | minutes |
| 0.50 | Polymerized within 5-10 | Polymerized within 5-10 |
| | minutes | minutes |

These results show that lyophilization in HCl and reconstitution of solubilized extracellular matrix compositions in 0.01 N HCl to 0.05 N HCl or in water maintains the capacity of the components of the compositions to polymerize. The results also show that lyophilization in acetic acid maintains the capacity of the components of the compositions to polymerize when the composition is polymerized in water or HCl. The solubility rate is lyophilization from 0.01 N HCl>lyophilization from 0.01 M acetic acid ≧lyophilization from water.

### Example 11

### Preparation of Solubilized SIS Composition

This procedure outlines a standard technique for the preparation of SIS solution.

1. Dissolution: of SIS powder in acetic acid with Pepsin
1.1. Preparation of acetic acid with pepsin
   1.1.1. Prepare the desired volume of 0.5 M acetic acid (typically IL; this requires 28.7 mL of 17.4 M glacial acetic acid).
   1.1.2. Add the desired mass of pepsin to achieve a 0.1% w/v solution (typically 1 g, if 1 L of acetic acid is used).
   1.1.3. Place the jar containing acetic acid and pepsin on a stir plate and begin mixing.
1.2. Preparation of centrifuged SIS powder
   1.2.1. Place SIS powder in 50 mL centrifuge tubes.
   1.2.2. Centrifuge SIS powder at 3000×g for 15 minutes.
   1.2.3. Open centrifuge tubes, pour off and dispose of supernate.
   1.2.4. Remove pellets from tubes. Measure out the desired mass to achieve a 5% w/v solution (typically 50 g, if 1 L of acetic acid was used). Previously prepared and frozen material may be used, and excess centrifuged material may be frozen for later use.
1.3. Add centrifuged SIS pellet material to acetic acid/pepsin solution.
1.4. Cover and allow it to stir for 72 hours at 4° C.

2. Centrifugation of dissolved SIS
2.1. When removed from stirring, the SIS/pepsin solution should appear viscous and somewhat uniform. Pour SIS/pepsin solution into centrifuge jars. Balance jars as necessary.
2.2. This mixture should be centrifuged at 16,000×g for 30 minutes at 4° C. Refer to the operators manual or SOP for instructions on using the centrifuge. If using the Beckman model J2-21, use the JIO head at a speed of 9500 rpm.
2.3. Remove jars of SIS from centrifuge. Pour the supernate into a clean jar. Be careful not to disturb the pellet, and stop pouring if the SIS begins to appear more white and creamy (this is pellet material).

3. Dialysis of SIS in water and hydrochloric acid
3.1. Prepare dialysis tubing as follows:
   3.1.1. Use dialysis tubing with MWCO 3500, diameter 291 mll. Handle dialysis tubing with gloves, and take care not to allow it to contact foreign surfaces, as it may easily be damaged.
   3.1.2. Cut dialysis tubing to the necessary length. (typically, 3 sections of about 45 cm).
   3.1.3. Wet tubing in millipore water, and leave tubing in the water until each piece is needed.
   3.1.4. Do the following with each length of tubing:
      3.1.4.1. Place a clip near one end of the tubing.
      3.1.4.2. Holding the tubing to avoid contact with foreign surfaces, use a pipette to fill the tubing with SIS solution. Each piece of tubing should receive roughly the same volume of SIS (for example, if three lengths of tubing are used, measure one third of the total volume into each).
      3.1.4.3. Place a clip on the open end of the dialysis tubing. Avoid leaving slack. The tube should be full and taut.
      3.1.4.4. Place the filled dialysis tubing in a container of 0.01 M HCL with a stir bar.
      3.1.4.5. Repeat the above steps to fill all lengths of tubing.
   3.1.5. Leave containers to stir at 4° C.
3.2. Details regarding changing the dialysis in 0.01 M HCl are given below.
   3.2.1. The 0.01 M HCl in the dialysis containers must be changed several times. This should be done as follows:
   3.2.2. After changing the 0.01 M HCl, another change should not be done for at least two hours.
   3.2.3. Change the 0.01 M HCl at least 10 times, over a period of at least four days. This assumes a ratio of 200 mL SIS to 6 L of 0.01 M HCl. If a higher ratio is used, more changes may be necessary.
   3.2.4. When changing 0.01 M HCl, do not leave dialysis bags exposed in the air or on the counter. Use tongs or forceps to move a dialysis bag directly from one container to another. (It is okay to have multiple dialysis bags in one container.) Dump the first container in the sink, then refill it with millipore water. The dialysis bags can now be placed in the newly filled container while the other container or containers are changed.

4. Sterilization of SIS
4.1. Place dialysis bags of SIS in a solution of 0.18% Peracetic acid/4.8% Ethanol. Leave to stir for two hours (more time may be necessary).
4.2. Translocate dialysis bags to 0.01 M HCl and continue dialysis as before.
   Continue for at least 2 days, changing HCl at least 3 times daily.
4.3. When dialysis is complete, dialysis tubing filled with SIS should be removed from the HCl.
4.4. Remove the clips. Cut open one and of the dialysis tubing and pour SIS into a clean jar.
4.5. SIS should be refrigerated until use.

5. Lyophilization of SIS
5.1. Operating the Vertis Freezemobile
   5.1.1. Make sure the condenser is fee of any water. (The condenser is the metal cylinder which opens on the front of the lyophilizer.) Ensure that the black rubber collection tubing attached to the bottom of the condenser is plugged. This can be accessed by opening the grate on the front of the lyophilizer.
   5.1.2. Close the door of the condenser, the top of the manifold and all sample ports. If the door of the condenser or the top of the manifold are not forming a good seal apply a small amount of vacuum grease to the rubber contact surfaces.
   5.1.3. Turn on the “Refrigerate” switch. The indicator on the front of the lyophilizer will show a light beside “Condenser” and beneath “On.” The light beneath “OK” will not illuminate until the condenser is cooled. The condenser temperature is indicated when the digital readout displays “C1.”
   5.1.4. When the “condenser” indicator light under “OK” is illuminated, on the “Vacuum” switch. The indicator will show a light beside “Condenser” and beneath “On.” The light beneath “OK” will not turn on until the chamber is sufficiently evacuated. The chamber pressure is indicated when the digital readout displays “V 1.”
   5.1.5. The rollers can be used for freezing a coat of material on the inside surface of a jar. To use the rollers, first ensure that the drain tube is plugged. (This can be accessed through the door on the right side of the front of the lyophilizer.) Using 100% Ethanol, fill the roller tank to a level several millimeters above the top of the rollers. Under-filling will cause ineffective cooling while over-filling will allow ethanol to leak into the jars. The temperature of ethanol bath is indicated when the digital readout displays “T1.” This bath is cooled when the “Refrigerate” switch is turned on. The “Rollers” switch controls the turning of the rollers, and may be switched off when no jar is on the rollers.
5.2. Lyophilizing SIS
   5.2.1. Lyophilization jars, glass lids, and rubber gaskets should be cleaned with ethanol. Allow ethanol to evaporate completely before use. Mid-size jars, lids, and gaskets (3-inch (7.62 cm) diameter) should be used to fit into the roller if using the Virtis Freezemobile Jar lyophilization.
   5.2.2. Pipette 75 mL of SIS solution into the lyophilization jar. Place gasket and lid on jar.
   5.2.3. Seal the jar by covering the openings with parafilm. Note the small hole on the neck of the lid, which must be covered.
   5.2.4. Place the jar of SIS on the lyophilizer rollers for a minimum of 2 hours.
   5.2.5. Alternatively, the jar may be placed in a freezer until all material is solid. In a −80° C. freezer, this takes about 30 minutes.
   5.2.6. Prepare a spigot on the lyophilizer by inserting a glass cock with the tapered end out. The tapered end of the cock should be coated with vacuum grease.
   5.2.7. Remove the jar of SIS from the rollers (or freezer). Place springs on the hooks to hold the jar and lid together. Remove the parafilm and place the neck of the lid of the jar over the cock. Rotate the jar so that the holes in the lid and the cock do not align. The spigot can be rotated so that the jar rests on the top surface of the lyophilizer.
   5.2.8. Turn the valve switch so that it points toward the jar of SIS.
   5.2.9. More jars may be added to freeze-dry simultaneously, but add jars one or two at a time. Wait until the vacuum pressure falls to a reasonable range (e.g., 200 millitorr) to ensure that the last jar is sealed before adding subsequent jars.
   5.2.10. Leave the jars under vacuum for at least 24 hours.
   5.2.11. After lyophilization is complete, turn the switch On the spigot to point away from the jar. This will allow air into the jar.
   5.2.12. Remove the jar from the cock.
   5.2.13. Lyophilized material is not immediately used, it should be stored in a dry environment. Use a large, sealable container with Dri-Rite or another desiccant, and place containers of lyophilized material therein.

6. Rehydration of lyophilized SIS
6.1. Place lyophilized SIS into a tube or jar.
6.2. Add the desired quantity of liquid (typically 0.01 N HCl) to the container of SIS.
6.3. Mixing may be accelerated by shaking, stirring, etc. Store container under refrigeration until dissolution of SIS is complete.
   Sterilization of Solubilized SIS by Dialysis against Peracetic Acid Containing Solution

1. Dialyze solubilized SIS against a large reservoir containing 0.18% peracetic acid/4.8% ethanol in water. Dialysis time may vary depending upon peracetic acid concentration, dialysis membrane molecular weight cut off, temperature, etc.

2. Transfer dialysis bags aseptically to reservoirs containing 0.01 N HCl. Dialyze extensively to reduce concentration of residual peracetic acid.

3. When dialysis is complete, dialysis tubing filled with solubilized SIS should be removed from the dialysis tank aseptically.

4. Remove dialysis clips and pour or pipette solubilized SIS into a sterile jar.

5. The disinfected solubilized SIS should be stored at 4° C. until use.

### Sterilization of SIS by Direct Addition of Peracetic Acid to SIS Solution

1. Add 100% Ethanol and 32 wt % peracetic acid to solubilized SIS to create a solution with final concentration of 0.18% peracetic acid/4.8% ethanol. Stir well and leave for two hours.

2. Place solubilized SIS in aseptic dialysis bags. Dialyze against sterile solution of 0.01 N HCl.

3. When dialysis is complete, dialysis tubing filled with solubilized SIS should be removed from the dialysis tank aseptically.

4. Remove dialysis clips and pour or pipette solubilized SIS into a sterile jar.

5. The disinfected solubilized SIS should be stored at 4° C. until use.

### Example 12

### Engineered ECM Compositions Regulate Cell Behavior

The three-dimensional (3D) extracellular matrix (ECM) of tissues in vivo represents a complex array of macromolecules that serves to provide biochemical and biophysical microenvironmental cues to resident cells. However, the exact role of any one biophysical feature or molecular component within the ECM in regulating cellular behavior has been difficult to elucidate due to the inherent interdependence of ECM compositional, structural, and mechanical properties. Recently, applicants have established that the 3D microstructural composition of fibrils within engineered ECMs created from purified type I collagen regulates cell-matrix adhesion, matrix remodeling, and proliferation properties of fibroblasts. It is further anticipated that altering the ratios of collagen types I and III within engineered ECMs would affect the hierarchical assembly of fibrils, and therefore the ECM signaling capacity.

Engineered ECMs were created with altered ratios of collagen types III and I ranging from 1:6 to 1:2. Application of confocal and scanning electron microscopy showed that ECMs prepared with increasing amounts of type III collagen possessed an increasing number of small diameter fibrils. Furthermore, these microstructural changes translated into alteration of matrix mechanical properties. Finally, results showed a biphasic response for fibroblast proliferation, morphology, and matrix remodeling.

### Example 13

### Engineered ECM Compositions Regulate Stem Cell Differentiation

A multipotential mesenchymal stem cell line (D1) derived from mouse bone-marrow stroma was obtained from American Type Culture Collection (ATCC). D1 cells were propagated in Dulbecco's modified Eagle medium containing 4.5 g/L glucose, 110 mg/L sodium pyruvate, 100 U/ml penicillin, 100 μg/ml streptomycin, and 10% fetal bovine serum (FBS) within a humidified atmosphere of 5% carbon dioxide at 37° C. Three-dimensional collagen ECMs were prepared by dissolving native, acid-solubilized type I collagen from calf skin (Sigma Chemical Co, St. Louis, Mo.) in 0.01 N hydrochloric acid to achieve desired concentrations. As a final purification step the isolated collagen obtained from Sigma Chemical was dialyzed against an acidic solution having a low ionic strength (0.01 N HCl) for 1-2 days, using dialysis tubing or a membrane having a molecular weight cut-off selected from a range of about 3,500 to about 12,000 daltons. For sterile preparations of collagen, the purified collagen solution was layered onto a volume of chloroform. After incubation for 18 hours at 4° C., the collagen solution layer was carefully removed so as not to include the collagen-chloroform interface layer.

To produce 3D purified collagen matrices with microstructures of varied collagen fibril dimensions (e.g., length, diameter, density), collagen solutions were polymerized under different conditions. Specifically, to create collagen matrices consisting of collagen fibrils at increasing densities, collagen solutions were polymerized at final collagen concentrations of 1.0 to 3.0 mg/ml. The polymerization composition comprised a 10× phosphate buffered saline (PBS) with an ionic strength of 0.14 N and a pH of 7.4. The specific formulation of the 10× phosphate buffer is as follows:

10×PBS, pH 7.4

1.37M NaCl

0.027M KCl

0.081M Na₂HPO₄

0.015M KH₂PO₄

5 mM MgCl₂

1% W/V glucose

To create 10×PBS buffers of different pH, the ratio of Na₂HPO₄ and KH₂PO₄ is varied. Ionic strength can be adjusted as an independent variable by varying the molarity of NaCl only. To create the 3D matrix comprising cells suspended within 3D matrix microenvironment the following components were mixed together:

1 ml solubilized collagen (e.g., type I collagen) in 0.01N HCl

150 ul 10×PBS, pH 7.4

150 ul 0.1N NaOH

100 ul 13.57 mM CaCl₂

100 ul 0.01 N HCl

Final Volume 1.5 ml.

The composition is mixed well after each additional component is added. The composition is then combined with a cell pellet of known cell number to create desired cell density; mixed well; and allowed to polymerize. The resulting polymerized 3D matrix has a final concentration of glucose and CaCl₂ of about 5.55 mM glucose and about 0.9046 mM CaCl₂.

To create collagen matrices consisting of collagen fibrils that varied in length and width, collagen solutions were polymerized at a pH selected from the range of 6.5-8.5. D1 cells were harvested in complete medium, collected by centrifugation, and added as the last component before polymerization. Tissue constructs were prepared at a relatively low cell density of 5×10⁴ cells/ml. Previous studies by applicants have shown that this cell density is suitable for maintaining cell viability, minimizing cell-cell interaction, and allowing the study of the dynamic relationship between an individual cell and its surrounding ECM.

Polymerization of tissue constructs was conducted in 24-well culture plates maintained in a humidified environment at 37° C. Immediately after polymerization (20 minutes or less), complete medium was added and the tissue constructs were cultured for 48 hours at 37° C. in a humidified environment consisting of 5% CO₂ in air. After 48 hours, each of the constructs comprising D1 cells seeded within a specific ECM microstructure were cultured under 3 different conditions:

1. complete medium no supplements

2. complete medium plus 10⁻⁷ M dexamethasone

3. complete medium plus 50 μg/ml ascorbic acid

For comparison purposes, parallel experiments were conducted on D1 cells grown in a standard 2D format on tissue-culture plastic. Cell behavior and morphology were monitored throughout the duration of the experiment using standard brightfield microscopy. After 24 days in culture, tissue constructs were histochemically stained with alcian blue, oil red 0, and alizirin red as indicators of chondrogenesis, adipogenesis, and osteogenesis.

Results:

The results of this experiment revealed the following:

1. multipotential stem cells seeded within engineered ECMs proliferated at rates that were dependent upon microstructural composition of the engineered ECM and the media composition;

2. time-dependent patterns of cellular condensation and aggregation exhibited by multipotential stem cells were dependent upon microstructural composition of the engineered ECM and the media composition;

3. time-dependent differentiation of multipotential stem cells seeded within engineered ECMs was dependent upon microstructural composition of the engineered ECM and the media composition;

4. maintenance of precursor or multipotential cells in an undifferentiated state in vitro was dependent upon microstructural composition of the engineered ECMs and the media composition;

5. patterns of cellular proliferation/differentiation for cells grown within 3D were different from those observed for cells grown in 2D on tissue culture plastic; and

6. decreasing the cell density of viable multipotential stem cells within engineered ECMs led to clonal growth of a large population of cells representing a single cell lineage. Estimates of optimal cell densities for clonal growth range from about 10 cells/ml to about 10³ cells/ml and depend upon the specific seeding efficiencies. For most cells, cell survival is known to decrease with seeding density.

### Example 14

### Engineered ECM Compositions Regulate Unipotential Stem Cell Differentiation

A unipotential stem (precursor) cell line (L1) derived from mouse and representing pre-adipocytes was obtained from American Type Culture Collection (ATCC). L1 cells were propagated in Dulbecco's modified Eagle medium containing 4.5 g/L glucose, 110 mg/L sodium pyruvate, 100 U/ml penicillin, 100 μg/ml streptomycin, and 10% fetal bovine serum (FBS) within a humidified atmosphere of 5% carbon dioxide at 37° C. To enhance cell viability, cells representing passage numbers greater than 5 were maintained in complete media in which the penicillin and streptomycin were reduced to 25 U/ml and 25 μg/ml, respectively.

Preparation of tissue constructs representing L1 cells seeded within 3D engineered ECMs of different microstructural compositions was carried out as described in Example 13. Immediately after polymerization (20 minutes or less), complete medium was added and the tissue constructs were cultured for 48 hours at 37° C. in a humidified environment consisting of 5% CO₂ in air. After 48 hours, each of the constructs comprising L1 cells seeded within a specific ECM microstructure were cultured under 3 different conditions:

1. complete medium no supplements; medium changed every 2 days thereafter;

2. complete medium no supplements and post differentiation medium treatment every 2 days thereafter; and

3. differentiation medium and post differentiation medium treatment every 2 days thereafter.

The differentiation medium consists of DMEM supplemented with 10% FBS, 25 U/ml penicillin, 25 μg/ml streptomycin, 115 μg/ml methyl-isobutyl xanthine, 10 μg/ml insulin, and 5×10⁻⁷M dexamethasone. The post differentiation medium consisted of DMEM supplemented with 10% FBS, 25 U/ml penicillin, 25 g/ml streptomycin, and 10 μg/ml insulin. For comparison purposes, parallel experiments were conducted on L1 cells grown in a standard 2D format on tissue-culture plastic. Cell behavior and morphology were monitored throughout the duration of the experiment using standard brightfield microscopy.

Results:

The results of this experiment revealed the following:

1. unipotential stem (precursor) cells seeded within engineered ECMs proliferated at rates that were dependent upon microstructural composition of the engineered ECM and the media composition;

2. time-dependent patterns of cellular condensation and aggregation exhibited by unipotential stem cells were dependent upon microstructural composition of the engineered ECM and the media composition;

3. time-dependent differentiation of unipotential stem cells into mature adipocytes seeded within engineered ECMs was dependent upon microstructural composition of the engineered ECM and the media composition;

4. maintenance of precursor cells in an undifferentiated state in vitro was dependent upon microstructural composition of the engineered ECMs and the media composition; and

5. patterns of cellular proliferation/differentiation for cells grown within 3D were different from those observed for cells grown in 2D on tissue culture plastic.

### Example 15

### Effect of Fibril Microstructure and Mechanical Properties of 3D ECM on Cultured Stem Cells

Multi-potential stem cells derived from the bone marrow of mice (D1s; ATCC) were suspended at 5×10⁴ cells/ml within purified type I collagen solutions (Sigma Chemical Co.) at varying collagen concentrations ranging from 1.5-3.6 mg/ml using the procedures described in Example 13. Tissue constructs consisting of D1 cells entrapped within a 3D ECM were formed by inducing self-assembly (polymerization) at pH 7.4, 137 mM NaCl, and 37° C. For this specific example, an increase in collagen concentration as a self-assembly parameter, was used to generate a 3D ECM microenvironment in which the density of the resultant fibrils and stiffness (linear or elastic modulus) of the matrix were systematically increased. The 3D constructs and resident cells were maintained in one of three different media formulations (Table 8) at 37° C. in a humidified environment consisting of 5% CO₂ in oxygen for periods of time up to 4 weeks. Basal medium consisted of Dulbecco's modified Eagle's medium supplemented with 4 mM L-glutamine, 4.5 g/L glucose, 15 g/L sodium bicarbonate, 1 mM sodium pyruvate, 10% fetal bovine serum, 100 U/ml penicillin, and 100 μg/ml streptomycin. For comparison purposes, D1 cells also were cultured in a parallel fashion in the standard 2D format on the surface of tissue culture plastic.

TABLE 8
Medium formulations used to culture D1 cells

| Medium | |
|---|---|
| Designation | Medium Formulation |
| A | Basal medium supplemented with 1 μM dexamethasone, |
| | 0.5 mM isobutylmethylxanthine, 1 μg/ml insulin |
| B | Basal medium supplemented with 0.1 μM dexamethasone, |
| | 8 μg/ml ascorbic acid, 5 mM β-glycerophosphate |
| C | Basal medium with no additives |

After various periods of time, the proliferative and differentiation status of the cells were determined qualitatively or quantitatively. Qualitative evaluation of cell number and morphology was conducted several times a week using light microscopy. Real-time RT-PCR was used to quantify and compare the expression levels of CFBA1 (runx2), LPL (lipoprotein lipase), and procollagen II as indicators of osteogenesis (bone formation), adipogenesis (fat formation), and chondrogenesis (cartilage formation), respectively. Histochemical stains, including alkaline phosphatase and oil red 0, were applied to whole mount or cryosectioned samples to detect osteogenic and adipogenic activity, respectively. In some cases immunohistochemical staining was used to corroborate results.

Cells grown in basal culture medium with no additives (medium formulation C) on standard tissue culture plastic (A) and within 3D ECM microenvironments of controlled fibril density and stiffness showed distinct growth patterns and morphologies. Results showed as the fibril density and stiffness of the 3D ECM microenvironment increased, the proliferative capacity of the cells decreased. The dependence of D1 proliferation on the stiffness of the 3D ECM microenvironment was noted for all media formulations studied. More specifically, D1 cells grown on plastic or within the low stiffness 3D ECM microenvironment showed an increased number of spindle-shaped cells. Within 2 weeks the cells on plastic reached confluence and formed a sheet of cuboidal shaped cells. On the other hand, spindle-shaped cells were evident within the low stiffness ECM even after 4 weeks of culture. These cells appeared to remain undifferentiated and populated the ECM uniformly. Growth patterns indicative of isolated clonogenic events were higher in frequency within ECMs of increased stiffness.

The observed differences in the growth patterns and morphologies adopted by cells grown in the 2D and 3D microenvironments suggested that the multi-potential cells were being directed down distinct differentiation patterns. Limited directed differentiation appeared to occur for cells grown on plastic or within the low stiffness ECMs (1.5 mg/ml). Interestingly, D1 cells grown within 3D ECMs of high stiffness (3.4 mg/ml) formed regional aggregates of cells indicative of osteogenesis and/or skeletal myogenesis. Osteogenesis but not myogenesis events were also observed with engineered ECMs of moderate stiffness (3 mg/ml). The biochemical composition of the media also could be varied to enhance the differentiation of cells down a specific pathway or to maintain cells in a relatively undifferentiated state. Specifically, cells grown in medium formulation A demonstrated a high frequency of adipogenesis on plastic and within 3D ECMs of low fibril density and stiffness (1.5 mg/ml). As the fibril density and stiffness of the 3D ECM microenvironment increased, adipogenesis events decreased and osteogenesis increased. Medium formulation B appeared to support differentiation of D1 cells into fat (adipogenesis) and (bone) osteogenesis on plastic. Limited areas of osteogenesis and adipogenesis were noted amongst a large number of spindle-shaped cells for D1 cells grown within ECMs of low stiffness under these same medium conditions. As the stiffness of the 3D ECM increased, cells more uniformly developed regional areas of osteogenesis and myogenesis-like events. A 2D projection of one confocal image revealed cells organized or fused to form a multi-cellular structure reminiscent of a myotube. These events were limited to 3D ECM microenvironments of high stiffness (3.4 mg/ml and greater). While these myotube-like events were noted in all three medium formulations, they appeared to occur more frequently in medium formulations B and C. The cells of the myotube-like structure were stained immunohistochemically for F-actin to demonstrate the fusion of and connectivity of the actin cytoskeleton between individual cells.

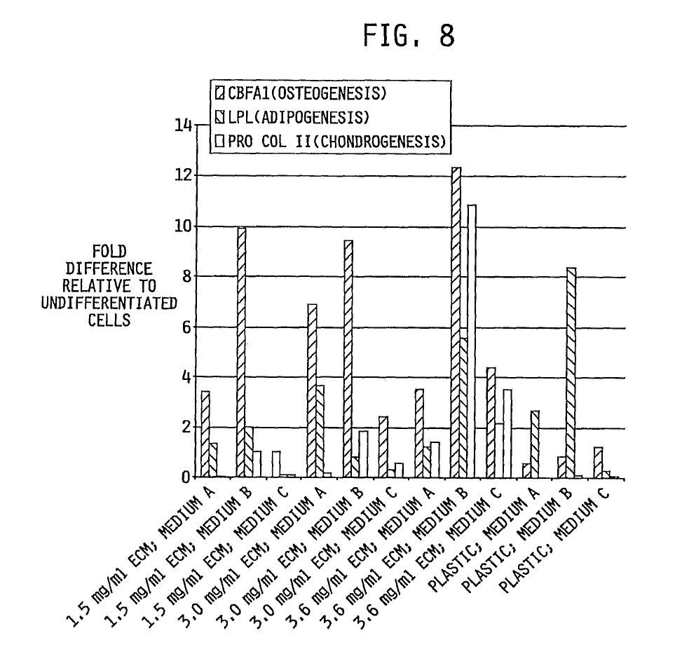

Real-time RT-PCR confirmed that biophysical features of the 3D ECM microenvironment (e.g., fibril density and ECM stiffness) could be modulated to regulate stem cell growth and differentiation. FIG. 8 shows the differences in gene expression patterns for D1 cells grown for two weeks on tissue culture plastic (Plastic) and within 3D engineered ECMs prepared at low (1.5 mg/ml), moderate (3.0 mg/ml), and high fibril density and stiffness (3.6 mg/ml). Again, cells subjected to each of these 2D and 3D culture formats were maintained in one of three different media formulations (Table 8).

The tissue specific genes CBFA1 (runx2), LPL (lipoprotein lipase), and Pro Col II (procollagen II) were selected as indicators of osteogenesis, adipogenesis, and chondrogenesis, respectively. Results showed that cells grown for 2 weeks on 2D plastic in the basal medium (no additives) remain relatively undifferentiated, more specifically, limited expression of the osteogenic, adipogenic, and chondrogenic indicators. On the other hand, D1 cells show an increase in LPL (adipogenesis) when cultured on plastic in the presence of Medium A or Medium B. The expression of LPL correlates well with the observed fat cell morphology developed within the cultures. Interestingly, the gene expression patterns developed by cells grown within a 3D ECM microenvironment were dramatically different from those observed for cells grown on plastic. Specifically, the expression of CBFA1 indicative of osteogenesis could be enhanced by growing the cells within 3D ECMs of increased stiffness or Medium B. Again, the increased expression of CFBA1 correlated well with cell morphologies and histochemical staining. Interestingly, chondrogenesis events as indicated by high procollagen II expression appeared to be enhanced within D1 cells cultured in 3D ECMs of high stiffness.

The starting cell density was also a critical determinant of the stem cell fate within the 3D culture formats studied. Clonal growth and cell differentiation events were favored by increasing the ECM stiffness and/or by decreasing the starting cell density within a given 3D ECM format. Adipogenesis was favored by decreasing the ECM stiffness and/or by increasing the cell density. Interestingly, adipogenesis was observed within high stiffness 3D ECMs only when the cell seeding density approached 1×10⁶ cells/ml and above.

### Example 16

### Cell Culture

Low passage neonatal human dermal fibroblasts (NHDFs) were obtained from Cambrex Bioproducts (Walkersville, Md.). NHDFs were propagated in fibroblast basal medium supplemented with human recombinant fibroblast growth factor, insulin, gentamicin, amphotericin-B, and fetal bovine serum (FBS) according to manufacturer's recommendations. Cells were grown and maintained in a humidified atmosphere of 5% CO₂ at 37° C. Cells representing a limited passage number of 20 or less were used for all experiments.

### Example 17

### Preparation of 3D Engineered ECMs and 3D Tissue Constructs

Purified type I and type III collagens, that were solubilized from bovine dermis and human placenta, respectively, were obtained from Sigma Chemical Company (St. Louis, Mo.). Three-dimensional engineered ECMs were prepared at a constant collagen type I concentration of 1.5 mg/ml and type III collagen concentrations of either 0, 0.25, 0.50, and 0.75 mg/ml (Table 9) using the general procedures described in Example 13. The polymerization buffer consisted of 10× phosphate buffered saline (PBS) with an ionic strength of 0.14 M and a pH of 7.4. All 3D engineered ECMs and tissue constructs were polymerized in vitro within a humidified environment at 37° C. To determine the cellular signaling capacity of each 3D ECM microenvironment, 3D tissue constructs were formed by first harvesting NHDFs in complete media and then adding the cells as the last component to the collagen solutions prior to polymerization. Tissue constructs were prepared at a relatively low cell density of 5×10⁴ cells/ml in order to minimize cell-cell interactions. Immediately after polymerization (20 minutes or less), complete medium was added and the tissue constructs were maintained at 37° C. in a humidified environment consisting of 5% CO₂ in air.

TABLE 9
Summary of formulations for 3D engineered ECMs prepared
with varied ratios of collagen types I and III.

| Type I | Type III | Total | | Type III |
|---|---|---|---|---|
| Collagen | Collagen | Collagen | Collagen Type | Collagen |
| (mg/ml) | (mg/ml) | (mg/ml) | I/III Ratio | (% of Total) |
| 1.5 | 0 | 1.50 | 0 | 0 |
| 1.5 | 0.25 | 1.75 | 6:1 | 14.3% |
| 1.5 | 0.50 | 2.00 | 3:1 | 25.0% |
| 1.5 | 0.75 | 2.25 | 2:1 | 33.3% |

A summary of the results of the data generated by the experiment of Example 13-17 is provided in FIG. 9.

### Example 18

### Preparation of Two-Dimensional (2D) ECM Surface Coatings

To prepare 2D surfaces coated with the different ECM compositions, solutions containing collagen type I (1.5 mg/ml) and varying concentrations of collagen type II (0, 0.25, 0.5, and 0.75 mg/ml) were aliquoted (300 μl/well) into tissue culture plates (24-well) and air-dried within a laminar flow hood for approximately 18 hours. Well plates containing 2D ECM surface coatings were equilibrated with PBS, pH 7.4, prior to seeding the NHDFs at a density of 2.5×10⁴ cells/well. Complete medium was added and the NHDFs on the surface of the 2D ECM coatings were maintained at 37° C. in a humidified environment consisting of 5% CO₂ in air.

### Example 17

### Qualitative and Quantitative Analysis of 3D ECM Microstructural Composition

Two quantitative parameters describing the 3D ECM microstructural composition, fibril area fraction (a 2D approximation of 3D fibril density) and fibril diameter, were determined based upon confocal reflection and scanning electron microscopy (SEM) images. Prior to microstructural analysis, engineered 3D ECM constructs were polymerized within four-well Lab-Tek coverglass chambers (Nalge Nunc International, Rochester, N.Y.) and placed within a humidified environment at 37° C. where they were maintained for approximately 15 hours. For measurements of fibril area fraction, the confocal microscope was used to obtain high resolution, 3D, reflection images of the component collagen fibrils within each ECM (Brightman at al., Biopolymers 54: 222-234, 2000; Voytik-Harbin et al., Methods Cell Biol 63: 583-597, 2001). Three images (at least 10 μm in thickness) were taken at random locations within each of 2 specimens representing a given 3D ECM composition. The confocal image stacks were then read into Matlab (The Mathworks, Natick, Mass.), and 2D projections, representing 21 z-sections, of each image were created and a threshold chosen for binarization. Using a built-in function in Matlab, the area occupied by collagen fibrils (white pixels) was calculated, converted to μm² based upon the pixel sizes, and normalized to the total image area.

Fibril diameter measurements were made by applying Imaris 4.0 (Bitplane Inc., Saint Paul, Minn.) to both confocal reflection and SEM images of engineered ECM constructs. For SEM imaging, engineered ECM constructs were fixed in 3% glutaraldehyde in 0.1M cacodylate at pH 7.4, dehydrated with ethanol, and critical point dried. Samples were sputter-coated with gold/palladium prior to imaging. Duplicate samples were imaged in a JEOL (Peabody, Mass.) JSM-840 SEM using 5 kV accelerating voltage and a magnification of 3,000×. Digital images were captured using 1280×960 resolution and 160 second dwell time. From each image obtained from duplicate samples, forty fibrils were chosen at random (10 fibrils per quadrant). Five lines were drawn perpendicular to the long axis of each fibril using the measurement tool in Imaris (Brightman at al., Biopolymers 54: 222-234, 2000). The average number of pixels representing the fibril diameter was then converted into □m based upon the known pixel size.

### Example 18

### Measurement of Tensile Mechanical Properties of 3D Engineered ECMs

Specimens for mechanical testing were prepared by polymerizing each soluble ECM formulation in a “dog bone” shaped mold as described previously (Roeder et al., J Biomech Eng, 124: 214-222, 2002). In brief, the mold consisted of a glass slide and a piece of flexible silicone gasket. The gauge section of the mold measured 10 mm long, 4 mm wide, and approximately 1.5 mm thick. Neutralized ECM solution was added to each mold and allowed to polymerize at 37° C. in a humidified environment where they were maintained for 18-20 hours prior to tensile loading. Polypropylene mesh was embedded in the ends of each 3D ECM construct to facilitate clamping for mechanical loading. Low-magnification, 4D images (x, y, z, and time) of each ECM construct during uniaxial tensile loading were acquired using an integrated mechanical loading-stereomicroscope set-up. This set-up involved interfacing a modified (Roeder et al., J Biomech Eng, 124: 214-222, 2002.) Minimat 2000 miniature materials tester (Rheometric Scientific, Inc., Piscataway, N.J.) with a Stemi 2000-C Stereomicroscope (Carl Zeiss MicroImaging; Thornwood, N.Y.) mounted with a DFC480 high-resolution color digital camera (Leica Microsystems, Cambridge, UK). Strategically placed right-angle prisms (Edmund Industrial Optics, Barrington, N.H.) were used to monitor changes in specimen thickness (z-direction) throughout the loading process. The image field was positioned to include the clamp that was attached to the load cell in order to provide a “fixed” frame of reference throughout the loading process. Each ECM construct was loaded uniaxially at an extension rate of 1 mm/min (corresponding to a strain rate of \dot{ε}≈0.04/min) until failure. Images were collected at a rate of 0.1 frames/sec to provide sequential images at 0.64% strain intervals. Changes in the width (x-direction) and thickness (z-direction) dimensions of the specimen's gauge section were measured directly from low-magnification digital camera images representing the width and thickness of the specimen and used to calculate cross-sectional area. The mechanical behavior of each specimen, including engineering stress (σₑ), true stress (σₜ), and applied strain (εₐₚ) were calculated from load-displacement recordings provided by the Mini-mat. Applied strain was calculated by simplifying the Lagrangian strain definition (Malvern, Introduction to the Mechanics of a Continuous Medium. Upper Saddle River, N.J.: Prentice-Hall, 1969) for a simple stretch λ (new length divided by original length) as indicated below

$${ɛ}_{ap} = \frac{1}{2} ( {λ}^{2} - 1 ) \qquad ( 1 )$$

Engineering stress was calculated as

$${σ}_{e} = \frac{F}{{A}_{o}} \qquad ( 2 )$$

where, F was the force recorded by the Minimat and Aₒ was the initial cross-sectional area (width×thickness) within the center of the specimen (Callister et al., Materials Science and Engineering: An Introduction. 3rd edition. New York, N.Y.: John Wiley & Sons, 1994). For calculation of true stress, the actual cross-sectional area of each specimen at a specific load was imaged, quantified, and substituted for Aₒ in the engineering stress equation above. From the resulting stress-strain relationships ultimate strength (maximum stress achieved during tensile loading), failure strain (strain at which specimen fails), and linear or elastic modulus (stiffness; slope of linear region of stress-strain curve) were determined.

### Example 19

### Multi-Dimensional Confocal Imaging of Cell-ECM Interactions

All multi-dimensional imaging was performed on a Bio-Rad Radiance 2100 MP Rainbow (Bio-Rad, Hemel Hempstead, England) multi-photon/confocal system adapted to a TE2000 (Nikon, Tokyo, Japan) inverted microscope with a heated stage set at 37° C. (ALA Scientific Instruments, Westbury, N.Y.). A custom-designed environmental chamber was adapted to the microscope to provide tissue constructs with a sterile environment of 5% CO₂ in humidified air (Pizzo et al., J Appl Physiol 98: 1909-1921, 2005). For each of the engineered ECMs studied, at least 5 individual cells were repeatedly monitored during the first 5 hours following construct polymerization. During the collection of time-lapse images, the confocal microscope was used in a reflection (back-scattered light) mode to obtain image stacks of an individual cell and the component collagen fibrils of its surrounding ECM as described previously (Brightman et al., Biopolymers 54: 222-234, 2000; Voytik-Harbin et al., Methods Cell Biol 63: 583-597, 2001). Images were collected at 30-minute intervals and a z-step of 0.5 μm to minimize exposure of the tissue constructs to radiation from the confocal microscope laser.

### Example 20

3D Cell Morphometric Analysis

Three-dimensional confocal images used for qualitative and quantitative analyses of NHDF morphology were collected using the confocal microscope in a combination reflection-epifluorescence mode (Voytik-Harbin et al., Methods Cell Biol 63: 583-597, 2001; Voytik-Harbin et al., Microsc Microanal 9: 74-85, 2003). Immediately following the 6-hr time-lapse imaging, tissue constructs were stained with the vital dye, Cell Tracker Green (Molecular Probes, Eugene, Oreg.), to facilitate discrimination of the cell from the surrounding collagen ECM. The processed image stack was used to determine fundamental morphological parameters including number of cytoplasmic projections, cell volume, 3D cell surface area, length, width, and height as described previously (Pizzo et al., J Appl Physiol 98: 1909-1921, 2005). Since each cell had a relatively unique orientation within the 3D matrix, these morphological parameters were defined based on a cellular coordinate system. Morphological evaluation was conducted on a total of 10 to 23 cells for each of the 3D ECM compositions studied.

### Example 21

### Determination of 3D Average Local Principal Strains and Points of Maximum Local Principal Strain

Consecutive time-lapse confocal reflection images representing the time-dependent deformation to the collagen fibril microstructure induced by an individual resident cell provided the basis for quantification of local ECM remodeling in terms of 3D displacements and strains. Strains were quantified using an incremental digital volume correlation (IDVC) algorithm developed previously by our laboratory (Roeder et al., J Biomech Eng 126: 699-708, 2004). To determine 3D average local principal strains within the surrounding ECM induced by an individual cell, first the 15×15×3 grid of displacements (174.2×174.2×24 μm³ total volume) from the IDVC algorithm were converted into 3D strains with x, y, and z directions based on the confocal coordinate system. The strains in the entire volume were then averaged in each of the three confocal directions to give a 3×3 symmetric matrix of average strains, ε_{avg}, such that

$${ɛ}_{avg} = [ \begin{matrix}{ɛ}_{xx} & {ɛ}_{xy} & {ɛ}_{xz} \\ {ɛ}_{xy} & {ɛ}_{yy} & {ɛ}_{yz} \\ {ɛ}_{xz} & {ɛ}_{yz} & {ɛ}_{zz}\end{matrix} ] \qquad ( 3 )$$

where εᵢⱼ are average strains in the confocal coordinate system directions. This average strain matrix in Equation (3) was then solved using eigenvector analysis (Strang et al., Linear Algebra and Its Applications. 3rd edition. San Diego, Calif.: Academic Press, 1988) to determine 3 average principal strains (E₁, E₂, E₃) and associated directions such that,

$${ɛ}_{avg} \cdot [ V ] = [ V ] \cdot [ \begin{matrix}{E}_{1} & 0 & 0 \\ 0 & {E}_{2} & 0 \\ 0 & 0 & {E}_{3}\end{matrix} ] \qquad ( 4 )$$

where [V] is a 3×3 matrix such that the column vectors (V₁, V₂, V₃) are the directions of the principal strains given by

[V]=[V₁ V₂ V₃] (5)

Therefore, the deformation induced by each cell had a unique set of average principal strains and directions in 3D.

Another analysis was performed to determine on a finer scale where the maximum local principal strains within the 3D ECM occurred in relationship to the cell. This analysis involved determination of local principal strains E₁, E₂, and E₃, each with unique principal direction, at each of the 15×15×3 grid points. The maximum compressive E₁, E₂, and E₃ were then identified within the image volume. The location of each maximum compressive principal strain was known in terms of its IDVC grid location and also in μm. The distance from these three-maximum principal strain locations to the center of the cell body in 3D could then be determined using simple vector relationships. The locations of the maximum compressive principal strains did not necessarily occur at the same grid locations for each cell.

### Example 22

### Labeling and Visualization of Actin Cytoskeleton within 3D Engineered Tissue Constructs

Tissue constructs formed by seeding NHDFs within specific 3D ECM formulations during polymerization were prepared in four-well Lab-Tek coverglass chambers (Nalge Nunc International, Rochester, N.Y.) for visualization of the F-actin cytoskeleton. At specified timepoints, constructs were fixed and permeabilized with a solution containing 0.1% Triton 100× and 3% paraformaldehyde, post-fixed in 3% paraformaldehyde, and treated with 1% bovine serum albumin to minimize non-specific binding. The constructs were then stained overnight at 4° C. with Alexa Fluor 488 Phalloidin (Molecular Probes, Eugene, Oreg.) and rinsed. Three-dimensional images of the F-actin distribution within an individual cell as well as its surrounding ECM were collected simultaneously using confocal microscopy in a combined epifluorescence and reflection mode. When necessary, images were deconvolved using AutoDeblur (Autoquant Imaging, Inc., Watervliet, N.Y.).

### Example 23

### Qualitative and Quantitative Determination of Cell Proliferation

Quantification of NHDF proliferation and its dependency on the 3D ECM microenvironment involved preparing 3D tissue constructs within 24-well tissue-culture plates using an alamarBlue-based proliferation assay as described previously (Pizzo et al., J Appl Physiol 98: 1909-1921, 2005; Voytik-Harbin et al., In Vitro Cell Dev Biol Anim 34: 239-246, 1998). For comparison purposes, the proliferative capacity of NHDF was also determined for an equivalent number of cells seeded directly onto the plastic surface of a well-plate as well as 2D plastic surfaces coated with different ECM compositions consisting of type I collagen in the presence of varying amounts of type III collagen. At time points representing 24 and 48 hours after construct polymerization and/or cell seeding, each well and tissue construct was examined microscopically to observe the viability, number, and morphology of the cells. The medium from each well then was replaced with fresh medium containing the metabolic indicator dye alamarBlue (10% v/v; BioSource International, Inc., Camarillo, Calif.). Twenty-four hours later, dye reduction was monitored spectrofluorometrically using a FluoroCount Microplate Fluorometer (Packard Instruments, Meriden, Conn.) with excitation and emission wavelengths of 560 nm and 590 nm, respectively. Background fluorescence measurements were determined from wells containing only dye reagent in culture medium. Maximum levels of relative fluorescence were determined from alamarBlue solutions that were autoclaved to induce complete dye reduction. The mean and the standard deviation values for all fluorescence measurements were calculated and subsequently normalized with respect to the background and maximum fluorescence readings. All experiments were performed in triplicate and repeated at least three times. When relevant, statistical analyses were performed using Matlab and included an analysis of variance (ANOVA). The Tukey-Kramer method for multiple comparisons (p<0.05) was then applied. The two-tailed t-test (α=0.05) was applied for pairwise comparisons.

### Example 24

### Three-Dimensional Microstructural Composition of Engineered ECMs Depends upon Collagen Type I/III Ratio

This study utilized the application of confocal microscopy in a reflection mode and SEM facilitated microstructural analysis from both 2D and 3D perspectives as well as at two different limits of resolution. SEM provided high-resolution (approximately 10 nm) 2D images of ECM microarchitecture after specimens had been critical point dried. On the other hand, confocal reflection microscopy allowed visualization of the 3D microstructural organization of component collagen fibrils within engineered ECMs in their fully hydrated or native state; however the resolution obtained with confocal imaging is approximately 200 nm, twenty times less than that obtained with SEM.

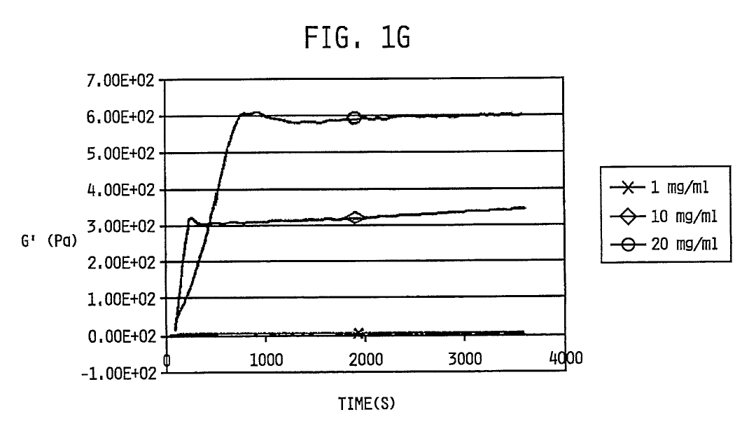

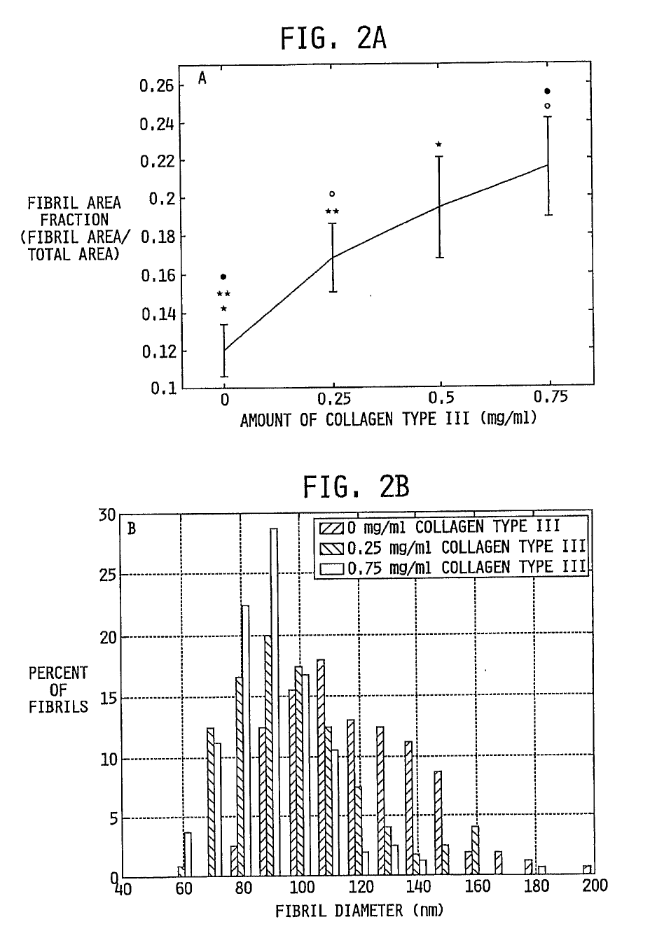

Both confocal and SEM images showed that ECMs prepared with increased amounts of type III collagen possessed an increased number or density of collagen fibrils. The fibril area fraction (FIG. 2A) was quantified from confocal reflection images and showed a nearly linear increase with type III collagen over the range studied. Engineered ECMs prepared from type I collagen alone had a fibril area fraction of 12.0±1.4% compared to 21.5±2.6% for those formed in the presence of the highest concentration (0.75 mg/ml) of type III collagen. In addition to this effect on fibril density, increased levels of type III collagen resulted in a downward shift in the fibril diameter distribution (Table 10) and (FIG. 2B). The mean fibril diameter as determined from SEM images for ECMs prepared with type I collagen alone was 115.2±23.2 μm. The mean fibril diameter showed a significant (p<0.05) decrease to 94.8±23.0 μm and 87.0±17.0 μm for ECMs in which collagen III was added at levels of 0.25 mg/ml and 0.75 mg/ml, respectively. In general, fibril diameter measurements made from confocal reflection images corroborated SEM results; however, fibril diameter values obtained from confocal images were greater than those obtained using SEM (Table 10) since confocal imaging was conducted on unprocessed, fully hydrated specimens. It should be noted that fibril diameter measurements made using confocal reflection imaging were considered somewhat less accurate and less precise since fibril diameters were near the limit of resolution for this imaging technique.

TABLE 10
Collagen fibril diameter measurement data for 3D engineered ECMs prepared from
type I collagen in the absence and presence of type III collagen as determined
from scanning electron (SEM) and confocal reflection (CRM) images.

| Fibril | | | Type I Collagen (1.5 mg/ml) + | |
|---|---|---|---|---|
| Diameter | Type I Collagen (1.5 mg/ml) | | Type III Collagen (0.75 mg/ml) | |
| (nm) | SEM | CRM | SEM | CRM |
| Mean ± SD | 115.16 ± 23.18 | 412.63 ± 76.35 | 87.04 ± 17.00 | 384.60 ± 71.96 |
| Median | 112 | 408 | 86 | 378 |
| Range | 78-194 | 200-664 | 56-176 | 236-628 |

### Example 25

### Mechanical Properties of Engineered ECMs Depend upon Collagen Type I/III Ratio

Previously, we showed that engineered ECMs prepared at increasing concentrations of type I collagen featured an increase in fibril density but no significant change in fibril diameter (Roeder et al., J Biomech Eng, 124: 214-222, 2002). Furthermore, this change in ECM microstructure, specifically an increase in collagen fibril density, was found to be positively correlated with ECM tensile strength and stiffness (linear or elastic modulus (Roeder et al., J Biomech Eng, 124: 214-222, 2002)). Traditionally, mechanical properties for collagen-based matrices have been calculated based upon engineering stress (Osborne et al., Med Biol Eng Comput 36: 129-134, 1998; Ozerdem et al., J Biomech Eng 117: 397-401, 1995; Roeder et al., J Biomech Eng, 124: 214-222, 2002), which assumes no change in specimen cross-sectional area during mechanical loading. However, since it is known that our engineered ECMs exhibit Poisson's ratios on the order of 2 to 4 (Roeder et al., J Biomech Eng, 124: 214-222, 2002; Voytik-Harbin et al., Microsc Microanal 9: 74-85, 2003), true stress was calculated to account for the significant reduction in cross-sectional area experienced by the scaffolds during testing. Since our experimental set-up facilitated the continuous monitoring of changes in specimen cross-sectional area during tensile loading, true stress calculated parameters were considered to most accurately reflect mechanical behavior of the ECMs.

ECMs engineered from type I collagen in the presence of type III collagen over the range of 0 to 0.75 mg/ml (type III collagen content of 0 to 33.3%) showed biphasic responses in terms of true stress calculated parameters ultimate strength and stiffness. The mean ultimate strength obtained for ECMs prepared from 1.5 mg/ml type I collagen alone was 136.7±49.9 kPa. Addition of collagen III resulted in significant reductions in ultimate strength, with 66.7±4.2 kPa (p=0.0016) and 75.1±22.7 kPa (p=0.0085) values being measured for ECMs prepared at collagen III levels of 0.25 mg/ml and 0.75 mg/ml, respectively. The linear or elastic modulus (stiffness), as determined from the linear region of the stress-strain curve, also showed reductions of 32% (p=0.0002) at the 0.25 mg/ml collagen III level and 18% (p=0.189) at the 0.75 mg/ml collagen III level compared to those where no collagen III was added. A decline in failure strain with increasing type III collagen content was noted. Specifically, failure strain values decreased significantly from 62.2±12.2% when no collagen III was added to 53.3±1.4% (p=0.048) and 43.0±5.9% (p=0.002) when the type III collagen content was 0.25 mg/ml and 0.75 mg/ml, respectively. Finally, increasing the type I collagen content from 1.5 to 3 mg/ml increased ECM ultimate strength and stiffness, confirming previous findings (Roeder et al., J Biomech Eng, 124: 214-222, 2002). ECMs prepared at 3 mg/ml type I collagen had ultimate strength and stiffness values that were 2.2 and 3.5 times, respectively, those obtained for ECM prepared at 1.5 mg/ml type I collagen.

### Example 26

### 3D Cell Morphology Depends upon Collagen Type I/III Ratio

The ability of cells to sense and respond to changes in the 3D ECM microenvironment that resulted from the addition of type III collagen initially was assessed by determining and comparing 3D cell morphology and cell-induced ECM remodeling (deformation and reorganization of component collagen fibrils). Three-dimensional morphometric analyses for cells seeded within the different ECM microenvironments were conducted at 6 and 12 hours following tissue construct formation. ECM remodeling by individual cells was repeatedly monitored during a 5 to 6 hour time window shortly after construct formation.

Notable differences in 3D cell morphometric parameters were detected at both 6 and 12 hours as the cells probed and adapted to their extracellular microenvironment.

One of the more prominent differences noted at 6 hours was that cells seeded within engineered ECMs prepared at the lowest type III collagen content (0.25 mg/ml) took on a rounded morphology with multiple short projections. This cell morphology contrasted that observed within ECMs prepared with 0.5 mg/ml and 0.75 mg/ml type III collagen. At these higher collagen III levels a large percentage of cells took on a more spindle-shaped cell body with fewer but prominent lengthy projections. Cells seeded within ECMs prepared from type I collagen alone took on a more spindle, bipolar shape and possessed the fewest (on average 2 to 4) but longest projections at the 6-hour time point.

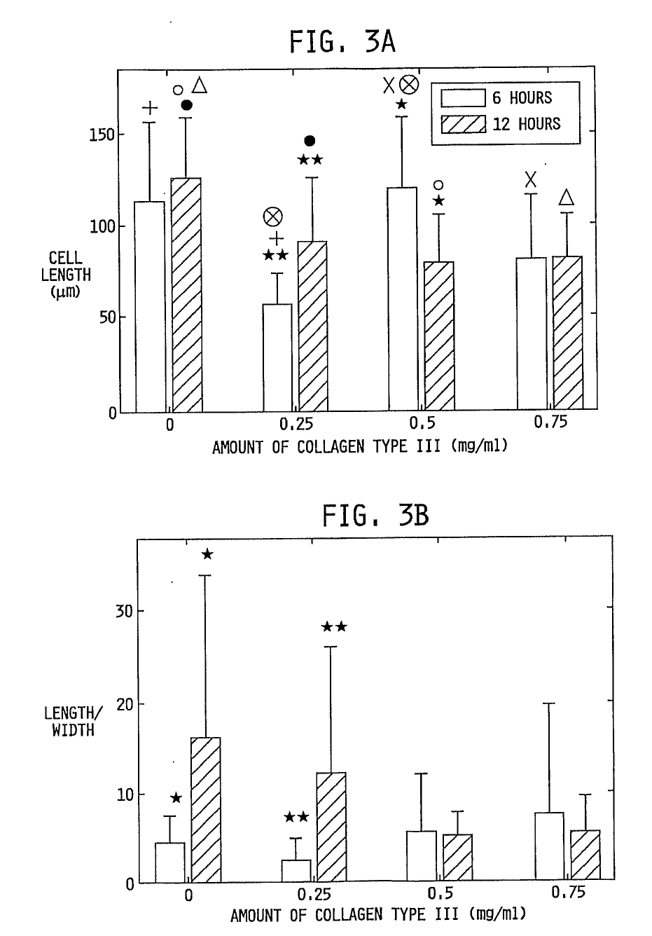

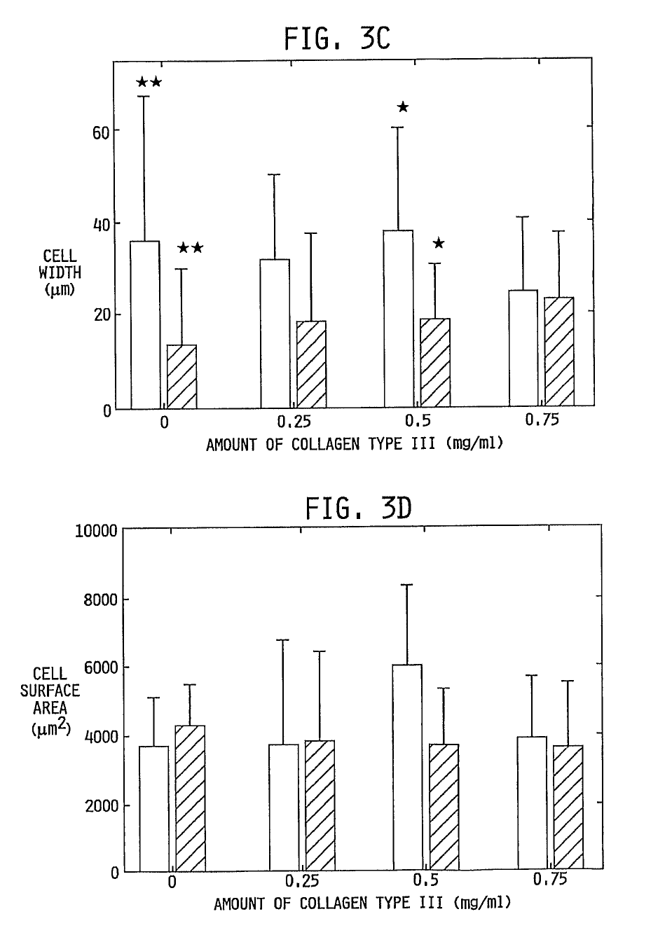

By 12 hours, the morphological differences that resulted from the various ECM microenvironments were subtler, largely owing to varying levels of ECM remodeling induced by cells at this time. At 12 hours, cells seeded within all ECM formulations appeared relatively spindle, bipolar-shaped. However, qualitative and morphometric analyses indicated that as the type III collagen content increased within the ECM, cells showed a statistically significant decrease in length (p<0.05), a subtle increase in width, and a decline in the length-to-width ratio as indicated (FIG. 3A-C). Based upon both qualitative and quantitative 3D morphology data, it appeared that cells grown in ECMs containing type III collagen took on a more contracted cell state. Despite the observed changes in cell shape, no significant differences were noted in 3D cell surface area (FIG. 3D) or cell volume at either time point. Consistent with previous studies (Pizzo et al., J Appl Physiol 98: 1909-1921, 2005), a larger proportion of cells grown within ECMs of higher total collagen content possessed an increased number of cytoplasmic projections at both 6- and 12-hour time points; however, this effect on projection number was less obvious when the collagen content was altered by adding type III collagen rather than type I collagen. Collectively these results demonstrate that cells adapt their shape, including the number and length of their projections, in response to ECMs that vary in collagen type I/III ratio. Furthermore, the morphological differences between cells appeared to be related to stiffness properties of the ECM.

### Example 27

### Collagen Type I/III Ratio of 3D Microenvironment Affects Contractile State of the Cell and ECM Remodeling

The collagen type I/III ratio also affected the ability of individual cells to deform and reorganize the component collagen fibrils of their surrounding ECM. Repeated monitoring of interactions between a cell and its surrounding collagen fibrils within a live tissue construct by confocal reflection microscopy provided a means of visualizing and quantifying this response over a 5 to 6 hour time window. An IDVC algorithm (Roeder et al., J Biomech Eng 126: 699-708, 2004) was applied to consecutive confocal image stacks and used to determine 3D displacements and strains as they occurred locally to a given cell and adjacent collagen fibrils. Data generated from this algorithm provided the basis for 1) quantification of volumetric strain induced by a single cell within a tissue construct; 2) a detailed analysis of average local principal strains for each imaged volume; and 3) determination of magnitudes and locations for points within the image volume where maximum principal strains, E₁, E₂, and E₃, occurred. This data was then compiled and used to compare the mechanical status of a large number of individual cells grown within the different ECM formulations.

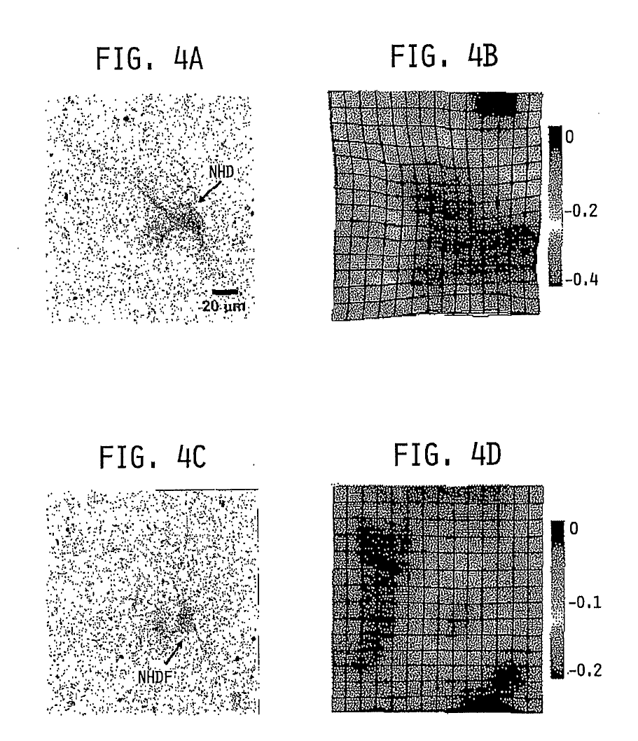

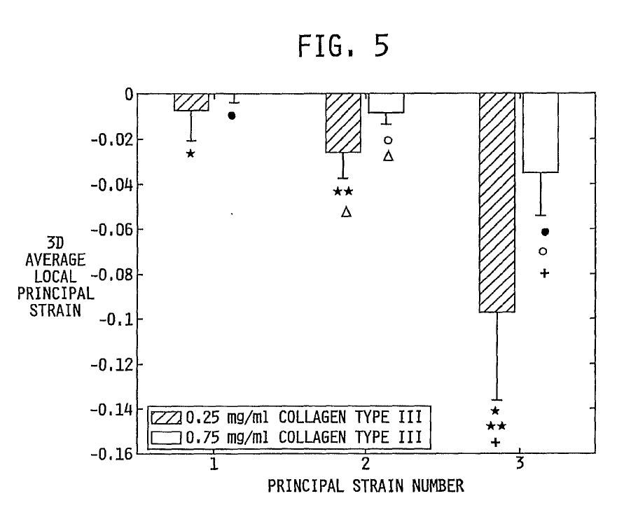

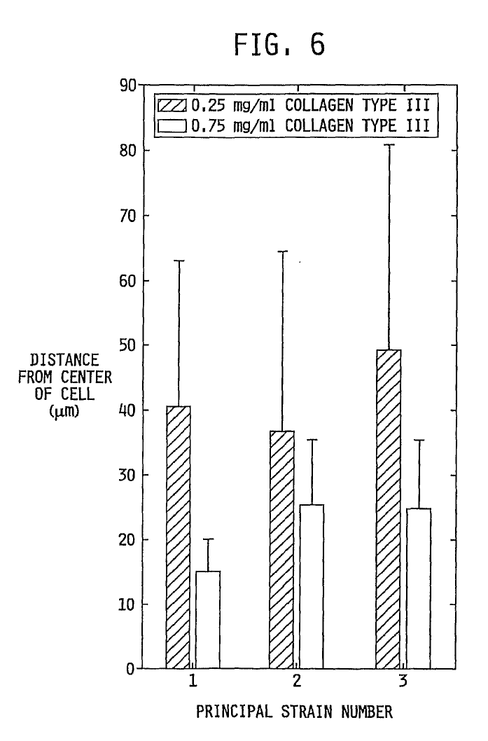

Results showed that cells grown in ECMs containing type III collagen were less able to contract and remodel the surrounding matrix as the type III collagen content increased or type I/III ratio decreased. Qualitative perspectives and corresponding volumetric strain data obtained for representative cells grown within type I collagen ECMs prepared with low (0.25 mg/ml) and high (0.75 mg/ml) type III collagen concentrations are shown (FIG. 4). Comparison of average local principal strains induced by cells grown within the different ECM formulations indicated that cells grown at low type III collagen levels (0.25 mg/ml) induced higher strain (approximately 3 to 3.5 greater) in each of the three principal directions compared to those grown at high type III collagen levels (0.75 mg/ml) and these differences were significant for E2 and E3 (p<0.05; FIG. 4). However, it is important to note that type III collagen containing ECMs with total collagen contents of 1.75 mg/ml to 2.25 mg/ml were characterized by 3D average local principal strain levels that were about 2 to 3 times greater than ECMs prepared of type I collagen alone and a total collagen content of 1 mg/ml. Analysis of the locations and magnitudes for points of maximum principal strain in the 1-, 2-, and 3-direction revealed that cells grown within engineered ECMs of type III collagen content of 0.25 mg/ml induced strain values that were approximately twice that exerted by cells grown within ECMs containing 0.75 mg/ml type III collagen (FIG. 5). Furthermore, in general, points of maximum principal strain for all three directions occurred at distances further from the center of the cell (FIG. 6) when grown in ECMs at the low versus high type III collagen content. Specifically, maximum principal strains were observed at distances of 40-50 μm from the center of the cell for ECMs containing 0.25 mg/ml type III collagen. However, cells within ECMs prepared with a type III collagen content of 0.75 mg/ml generated maximum principal strains at distances of only 15-25 μm from the center of the cell. Although the addition of type III collagen enabled cells to induce large principal strains within their ECMs, the distance at which maximum principal strain occurred was considerably less than that found for ECMs prepared at low levels of type I collagen alone (FIG. 6). More specifically, ECMs prepared at 1 mg/ml type I collagen yielded, on average, points of maximum principal strain for 1-, 2-, and 3-directions at distances of 48 μm, 45 μm, and 52 μm from the center of the cell, respectively. It was also noted that the locations of the maximum principal strain were often associated with and occurred along major cell projections, especially for cells grown at the high collagen type I/III ratios. Furthermore, fibril deformation patterns were dependent upon the collagen type I/III ratio. Remodeling of ECMs containing type III collagen was characterized by fibril condensation around the cell periphery. On the other hand, ECMs prepared from type I collagen alone showed regional areas of fibril alignment. The difference in ECM remodeling, as indicated by both qualitative fibril deformation and quantified strains suggested differences in mechanical properties between fibrils formed from homotypic type I and heterotypic type I/III fibrils.

Based collectively on the observed effects of type III collagen on ECM microstructure/mechanical properties as well as differences in the 3D cell morphology and cell-induced ECM remodeling within these matrices, it was hypothesized that varying the collagen type I/III ratio within the ECM microenvironment modulated the contractile state of resident cells. To test this hypothesis, cells were seeded within the 3D ECM microenvironments and the organization of cytoskeletal actin was visualized using confocal microscopy 6 hours post polymerization. Results showed prominent actin stress fiber formation for cells within ECMs containing collagen III. Well-organized actin bundles (stress fibers) were even observed within ECMs containing the highest collagen III concentration, 0.75 mg/ml, despite the high total collagen content and fibril density. Cells containing a few scattered actin filaments were observed in ECMs prepared from type I collagen alone, but only at low collagen concentrations of 1.5 mg/ml and below. Cells with diffuse actin staining patterns were noted within ECMs prepared at collagen I levels greater than 1.5 mg/ml. Diffuse actin staining patterns were observed for cells grown in engineered ECMs representing type I collagen ECMs prepared at concentrations of 1 mg/ml and 3 mg/ml. A few organized actin bundles were noted in engineered ECMs created from 1 mg/ml type I collagen and a large number of organized actin bundles running parallel along the major cytoplasmic projections or long axis of cells grown within engineered type I collagen ECMs formed in the presence of 0.75 mg/ml type III collagen. Collectively, results showed that stress fiber formation, which is indicative of the contractile state of the cell, was positively related to cell-induced local ECM remodeling and strain and inversely related to ECM stiffness.

### Example 28

### Collagen Type I/III Ratio within 3D Engineered ECMs but not 2D ECM Surface Coatings Modulates Cellular Proliferation

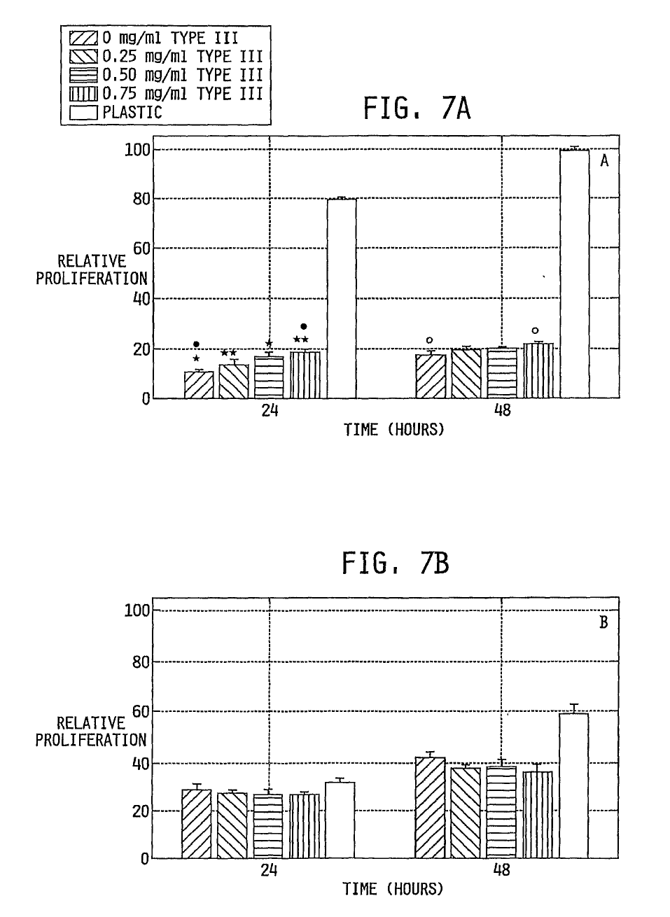

To determine the effect of the collagen type I/III ratio on the fundamental proliferative behavior of cells, NHDFs were seeded within the different ECM formulations. For comparison purposes, parallel studies were conducted in which fibroblasts were seeded onto tissue culture plastic. The number of living cells present at 24 and 48 hours following cell seeding was quantified indirectly using the metabolic indicator dye alamarBlue and confirmed qualitatively. Consistent with previous studies (Pizzo et al., J Appl Physiol 98: 1909-1921, 2005), cells grown within a 3D ECM microenvironment proliferated at decreased rates compared to those grown in a 2D format on tissue culture plastic (FIG. 7A). Fibroblast proliferation was enhanced in ECMs with increased type III collagen content (FIG. 7A). Since the type I collagen content was kept constant, increasing the amount of type III collagen also increased the overall collagen content. Although the total number of cells within all ECM formulations increased between 24 and 48 hours, the total number of fibroblasts was greatest for ECMs prepared with the highest type III collagen concentration for both time points. When type III collagen was added at levels below 0.25 mg/ml, in the range of 0.02 mg/ml to 0.10 mg/ml, the proliferative capacity of the resident cells was lower than that obtained for 1.5 mg/ml type I collagen alone.

Since the addition of type III collagen affected not only microstructural-mechanical properties but also the macromolecular composition of the engineered ECMs, it was uncertain if changes in NHDF proliferation were a result of differences in biophysical or biochemical signals (cues) inherent in the 3D ECM microenvironments. To isolate the biochemical and biophysical variables, traditional experimental methods involving creation of 2D ECM surface coatings consisting of varied collagen I/III ratios to evaluate cell-ECM interactions were applied. NHDF were seeded onto the ECM-coated surfaces and proliferation monitored. No significant difference was observed in cell proliferation due to type III collagen content at either the 24- or 48-hour time points (FIG. 7B). All coatings showed a significant increase (p<0.05) in cell number between the 24- and 48-hour time points. And at the 48-hour time point, cells seeded on plastic showed significantly greater proliferation than those seeded on any of the ECM coated surfaces p<0.05).

### Example 29

### Comparison of Structure-Function of Engineered ECM Formulations

Various engineered ECM formulations were compared to analyze three-dimensional microstructure-mechanical properties, including fibril area fraction, fibril diameter, and stiffness of the engineered ECM (Table 11). The various engineered ECM formulations were also compared in regards to ECM contraction, morphology, and cell proliferation (Table 11).

TABLE 11
Comparison of Structure-Function
of Engineered ECM Formulations

| Engineered ECM Formulation | | | |
|---|---|---|---|
| | | | 1.5 mg/ml |
| | | | Type I + |
| 1.0 mg/ml | 1.5 mg/ml | 3 mg/ml | 0.75 mg/ml |
| Type I | Type I | Type I | Type III |

3D ECM Microstructure-Mechanical Properties

| | | | | |
|---|---|---|---|---|
| Fibril Area | + | ++ | +++ | +++ |
| Fraction | | | | |
| (Density) | | | | |
| Fibril | ++ | ++ | ++ | + |
| Diameter | | | | |
| Stiffness | + | ++ | +++ | + |

Cellular Response:
ECM Contraction/Morphology/Proliferation

| | | | | |
|---|---|---|---|---|
| ECM | +++ | ++ | + | ++/+++ |
| Contraction | | | | |
| Distance | +++ | +++ | ++ | + |
| Number of | + | ++ | +++ | + |
| projections | | | | |
| Length of | Medium- | Medium- | Medium- | Short |
| projections | Long | Long | Long | |
| Morphology | Long- | Long- | Stellate | Short- |
| | Spindle | Spindle | | Spindle |
| Cytoskeletal | Stress- | Stress- | Diffuse | Stress- |
| Actin | fibers | fibers | | fibers |
| Proliferation | ++ | ++ | + | +++ |

### Example 30

Recent studies have demonstrated that human adipose-derived stem cells (ASC) derived from adult human adipose tissue secrete bioactive levels of multiple angiogenic and antiapoptotic growth factors including granulocyte-macrophage colony stimulating factor (GM-CSF), VEGF, hepatocyte growth factor (HGF), bFGF, and transforming growth factor-β (TGF-β), and are able to enhance blood flow and minimize death of ischemic muscle tissue [Rehman et al., 2004, Circulation 109: 1292-8]. These results are important because they indicate that autologous delivery of ASC, which are readily available from liposuction under local anesthesia, may be a novel and uniquely feasible therapeutic option to enhance angiogenesis and tissue rescue in ischemia. However, quantitative analysis of cell delivery has documented that the majority of peripheral blood mononuclear cells or ASC injected via intramyocardial, intracoronary, and interstitial retrograde coronary venous (IRV) in an ischemic swine model are not retained in the heart immediately following delivery and that the processes of delivery were highly inconsistent. In addition, examination of ASC surviving 1 week following intramuscular injection showed reduction of cell numbers to 25% of the injected cells over this period, suggesting limited cell survival [Rehman et al., 2004, Circulation 109: 1292-8]; this is further corroborated in the myocardial system by survival of approximately 20% or less of initially retained mesenchymal stem cells over 4 weeks post-injection.

The survival, proliferation, and differentiation properties of human APC and EPC cells implanted within three dimensional matrices will be investigated using both standard cell culture media or by suspension in any of the formulations of “ready-to-assemble” components of self-assembling 3D matrix microenvironments, in which the microstructure, composition, and mechanical properties are quantified and systematically varied. The delivery efficiency and subsequent engraftment (cell survival and differentiation) of human ASC or endothelial progenitor cells derived from human cord blood (EPC) implanted within an animal model of hindlimb muscle ischemia will also be investigated. More particularly, cells will be delivered with or without injectable 3D matrix microenvironments in which the “instructive” or signaling properties are controlled and systematically varied.

### Methods:

A series of in vitro experiments will be conducted to determine the effect of specific biophysical features of a cell's 3D ECM microenvironment on the fundamental behavior of human adipose-derived stem cells (ASC) and highly proliferative endothelial progenitor cells derived from human cord blood (EPC). ASC will be harvested from human adipose tissue as described previously [Rehman et al., 2004, Circulation 109: 1292-8]. Cultures of endothelial progenitor cells will be obtained from umbilical veins using established procedures [Ingram et al., 2004, Blood 104:2752-2760]. 3D ECM microenvironments in which specific biophysical features including fibril density, length, and width and stiffness are systematically varied will be created from purified collagen as described previously [Pizzo et al., 2005, J. App. Phys. 98:1909-1921; Roeder et al., 2002 J. Biomech Eng. 124: 214-22213,17]. In addition, 3D microenvironments in which composition is systematically varied by including ECM molecules such as type III collagen, hyaluronic acid, VEGF, bFGF will also be investigated. These molecules were chosen based upon their known role in neovascularization and cardiac muscle development. In all cases, cells will be added as the last component of the solubilized collagen matrix and the suspension will be injected over 30 seconds through a 25 Ga needle (paralleling intramuscular injection for in vivo systems) into a well plate and polymerized at 37° C. Immediately following polymerization (less than 30 minutes), complete medium will be added to all constructs.

For these studies, cell seeding densities ranging between 1×10 to 1×10⁷ cells/ml will be evaluated. Fundamental cell behaviors including survival, morphology, proliferation, and differentiation will be determined using techniques established previously [Pizzo et al., 2005, J. App. Phys. 98:1909-1921]. In some cases, cells will be prelabeled with CellTracker dyes or transfected with GFP and analyzed in 3- or 4-dimensions using confocal microscopy in a combination reflection-fluorescence mode. Outcomes will be compared to those from control “deliveries” in which cells are injected into media within culture plates, parallel to the situation for cell injection into a tissue environment in the absence of a solubilized, self-assembling matrix.

In addition to the in vitro culturing of cells within the 3D microenvironments, the 3D cell containing matrices will be implanted via injection into either normal or ischemic muscle in vivo, using the hindlimb model of muscle ischemia that the March lab has established and published in the preliminary findings concerning adipose stem cells [Rehman et al., 2004, Circulation 109: 1292-8]. Briefly, nude mice are employed so that cells of human origin can be studied in the absence of xenogeneic barriers. The ilio-femoral artery is surgically ligated and excised as described previously, in the left hindlimb only. The right hindlimb thus serves as a non-ischemic control. The musculature of the distal legs (e.g., tibialis anterior) then can be used as a well-demarcated delivery site for 100 μl injections into normal (right) and ischemic (left) muscle, that are performed under direct visualization. Injections of precisely defined numbers of ASC or EPC will be conducted 1 day following the surgical induction of ischemia in mice, with groups of 5 animals for each condition to be evaluated. The conditions will include control injections in saline (the previous standard) or in soluble self-assembling matrices. The cells will be labeled with GFP to permit enumeration by subsequent flow cytometry following muscle dissociation, as well as microscopic evaluation of the anatomy of engraftment and differentiation in selected mice. Mice injected will be sacrificed at either 3 hours post-injection, to quantify the number of cells retained acutely following delivery; and at 2 weeks post-injection, to determine precisely the cell survival over time following the injection. Cells will be counted by flow cytometry with the addition of fluorescent particles to permit precise volumetric enumeration. A total of 60 mice will be used in this study (e.g., 2 cell types×3 ECMs×5 animals/group×2 timepoints). The key endpoints will be quantitation of cell retention, and subsequent survival and engraftment into muscle or vasculature in the normal and ischemic muscles.

### Example 31

### Effect of Hyaluronic Acid Content in 3D Matrices on Cell Behavior

### Materials and Methods

Cell culture.

Low passage neonatal human dermal fibroblasts NHDFs), growth media, and passing solutions were obtained from Cambrex Bioproducts (Walkersville, Md.). NHDF were propagated in fibroblast basal medium supplemented with human recombinant fibroblast growth factor, insulin, gentamicin, amphotericin B, and FBS according to manufacturer's recommendation. Cells were maintained in a humidified atmosphere of 5% CO₂ at 37° C. and cell passage numbers representing 15 or less were used for all experiments.

### Engineered 3D Tissue Constructs.

To investigate the effect of hyaluronic acid (HA) on ECM assembly and signaling, type I collagen matrices with varied HA concentrations were prepared. Native (acid solubilized) type I collagen prepared from calf skin (Sigma) and hyaluronic acid prepared from bovine vitreous humor (Sigma) were each dissolved in 0.01 N hydrochloric acid (HCl) to achieve desired concentrations. Dissolved collagen was sterilized by exposure to chloroform overnight at 4° C. Three-dimensional engineered ECMs were prepared similar to those described in Example 13 at a constant collagen type I concentration (2 mg/ml) and hyaluronic acid concentrations of between 0 and 1.0 mg/ml. The polymerization buffer consisted of 10× phosphate buffered saline (PBS) with an ionic strength of 0.14 M and a pH of 7.4. All 3D engineered ECMs and tissue constructs were polymerized in vitro within a humidified environment at 37° C. To determine the cellular signaling capacity of each 3D microenvironment, 3D tissue constructs were formed by first harvesting NHDFs in complete media and then adding the cells (5×10⁴ cells/ml) as the last component to the collagen solutions prior to polymerization. Immediately following polymerization complete media was added and the constructs were maintained in a humidified atmosphere of 5% CO₂ in air at 37° C.

### Qualitative and Quantitative Analysis of 3D ECM Microstructure

Two quantitative parameters describing the 3D fibril microstructural composition of the ECM, fibril area fraction (a 2D approximation of 3D fibril density) and fibril diameter, were determined based upon confocal reflection and scanning electron microscopy (SEM) images. Prior to microstructural analysis, engineered 3D ECM constructs were polymerized within four-well Lab-Tek coverglass chambers (Nalge Nunc International, Rochester, N.Y.) and placed within a humidified environment at 37° C. where they were maintained for approximately 15 hours. For measurements of fibril area fraction, the confocal microscope was used to obtain high resolution, 3D, reflection images of the component collagen fibrils within each ECM. Three images (at least 10 μm in thickness) were taken at random locations within specimens representing a given 3D ECM composition. The confocal image stacks were then read into Matlab (The Mathworks, Natick, Mass.), and 2D projections of each image were created and a threshold chosen for binarization. Using a built-in function in Matlab, the area occupied by collagen fibrils (white pixels) was calculated, converted to μm2 based upon the pixel sizes, and normalized to the total image area.

Fibril diameter measurements were made by applying Imaris 4.0 (Bitplane Inc., Saint Paul, Minn.) to both confocal reflection and SEM images of engineered ECM constructs. For SEM imaging, engineered ECM constructs were fixed in 3% glutaraldehyde in 0.1M cacodylate at pH 7.4, dehydrated with ethanol, and critical point dried. Samples were sputter-coated with gold/palladium prior to imaging. Samples were imaged in at least duplicate with a JEOL (Peabody, Mass.) JSM-840 SEM. From each image obtained, twenty fibrils were chosen at random (5 fibrils per quadrant). Five lines were drawn perpendicular to the long axis of each fibril using the measurement tool in Imaris. The average number of pixels representing the fibril diameter was then converted into μm based upon the known pixel size.

### Dynamic Mechanical Testing of 3D Engineered ECMs

Mechanical properties of the engineered ECMs were measured using a TA Instruments (New Castle, Del.) AR-2000 rheometer. Soluble ECM preparations were adjusted to specific polymerization conditions and placed on the peltier temperature-controlled lower plate at 22° C., and the 40-mm parallel-plate geometry was lowered to a 1-mm gap. The temperature was then raised to 37° C. to initiate polymerization. The peltier heated plate required about 1 minute to stabilize at 37° C. Measurements of storage modulus G′ and loss modulus G″ of the polymerizing material under controlled-strain oscillatory shear were made every 30 seconds under oscillation at 1 Hz and 0.1% strain for a proscribed time. This strain was sufficiently small to ensure that it did not affect the kinetics of polymerization. Two hours and thirty minutes after polymerization, a shear creep test was conducted with a shear stress of 1 Pa for 120 seconds. Creep data was interpreted with a standard four-element Voigt spring dashpot model. Next a frequency sweep of controlled-strain oscillatory shear was made at 0.1% strain, from 0.01 to 20 Hz. Following the frequency sweep, a continuous shear stress ramp from 0.1 to 10.0 Pa over 2 minutes was applied. Finally, the specimen was subjected to unconfined compression at a rate of 10 μm/sec.

### Qualitative and Quantitative Determination of Cell Proliferation

Quantification of NHDF proliferation and its dependency on the 3D ECM microenvironment involved preparing 3D tissue constructs within 24-well tissue-culture plates. For comparison purposes, the proliferative capacity of NHDF was also determined for an equivalent number of cells seeded directly onto the surface of tissue culture plastic. At timepoints representing 24 and 48 hours after construct polymerization and/or cell seeding, each well and tissue construct was examined microscopically to observe the viability, number, and morphology of the cells. The medium from each well then was replaced with fresh medium containing the metabolic indicator dye alamarBlue (10% v/v; BioSource International, Inc., Camarillo, Calif.). Approximately 18 hours later, dye reduction was monitored spectrofluorometrically using a FluoroCount Microplate Fluorometer (Packard Instruments, Meriden, Conn.) with excitation and emission wavelengths of 560 nm and 590 nm, respectively. Background fluorescence measurements were determined from wells containing only dye reagent in culture medium. Maximum levels of relative fluorescence were determined from alamarBlue solutions that were autoclaved to induce complete dye reduction. The mean and the standard deviation values for all fluorescence measurements were calculated and subsequently normalized with respect to the background and maximum fluorescence readings.

### Time-Lapse Imaging of Cell-ECM Interactions

Tissue constructs representing NHDFs seeded at 5×10⁴ cells/ml within 3D engineered ECMs with defined microstructural and biochemical compositions were evaluated using time-lapse confocal microscopy. Beginning 1 hour after polymerization, 2 to 3 cells were repeatedly monitored using the confocal microscope in a reflection (back-scattered light) mode to obtain image stacks of the individual cell and its surrounding matrix as described previously (Voytik-Harbin, et al., Microscopy and Microanalysis, 9:74-85, 2003). Images were collected at 30-minute intervals and a z-step of 0.5 mm to minimize exposure of the tissue constructs to radiation from the argon laser.

### Determination of Volumetric Strain

Consecutive confocal reflection images representing temporal deformation induced by a resident cell on its surrounding ECM microstructure provided the basis for the quantification of local displacements and strains in 3D. Within each image, subvolumes of 32×32×20 pixels in the x, y, and z directions, respectively, were established. Each subvolume represented a group of voxels centered around a given point at which displacement values were sought. Each image subvolume provided a unique 3D voxel intensity pattern that allowed correlation pattern matching between consecutive images using an incremental digital volume correlation (IDVC) algorithm developed previously by our laboratory (Roeder et al., J. Biomech. Eng. vol. 124, pp. 214-222 (2002)). The IDVC algorithm provided strain-state data, including principal strains and their associated directions, for all grid point locations. Grid points were established in 512′512-pixel images that were 32 pixels apart in both x- and y-directions, with 24-pixel spacing in the z-direction. Principal strains determined for the length (E_{L}), width (E_{W}), and height (E_{H}) directions were used to calculate volumetric strain (E_{V}) based upon the following formula:

E_{V}=E_{H}+E_{W}+E_{L}+(E_{W}·E_{H})+(E_{L}·E_{H})+(E_{L}·E_{W})+(E_{L}·E_{W}·E_{H})

### Determination of 3D Cell Morphology

Prior to imaging at either 6 or 12 hours after construct polymerization, tissue constructs were stained with the vital dye Cell Tracker Green (Molecular Probes, Eugene, Oreg.) to facilitate discrimination of the cell from the surrounding collagen ECM. Confocal image stacks were then collected in a combined reflection-epifluorescence mode for determination of cell morphology and fibril microstructural organization.

### Results

Fibril diameter distribution was measured, as determined from scanning electron microscopy images, for engineered matrices prepared from type I collagen in the presence of varied amounts of hyaluronic acid. Over the range of hyaluronic acid concentrations tested, no significant difference was observed in mean fibril diameter. Mean fibril diameter measurements were 80.8±18.3 μm, 72.2±13.0 μm, and 72.1±11.8 μm (±standard deviation) for engineered matrices prepared from 2 mg/ml type I collagen containing 0, 0.5 mg/ml, and 1.0 mg/ml hyaluronic acid, respectively. Interestingly, it did appear that the variation (standard deviation) of fibril diameter measurement decreased with increasing hyaluronic acid content. No observable or quantitative differences in fibril area fraction measurements were determined for the engineered matrices prepared with and without hyaluronic acid.

While hyaluronic acid did not dramatically effect the fibril microstructure of engineered matrices, the polymerization rate was found to decrease with increasing hyaluronic acid content as indicated by a decreased slope of the G′ versus time plot. Furthermore, as the hyaluronic acid content increased, the engineered matrices showed an increase in compliance and an increase in their compressive stiffness, respectively. These results demonstrate that the 3D fibril microstructure as well as the viscous fluid component provide critical determinants of the overall mechanical properties of the engineered ECMs.

Studies comparing the cell response to 3D ECM microenvironments prepared with various hyaluronic acid content have shown no significant difference in the proliferation properties of neonatal human dermal fibroblasts. However, analysis of cell morphology and matrix contraction (remodeling) by cells indicate that hyaluronic acid alters the mechanics of cell-ECM interactions. Analyses of the magnitude and spatial distribution of local, 3D strain induced by a resident cell within an engineered matrix microenvironment revealed that the addition of hyaluronic acid reduces the ability of fibroblasts to effectively contract and induce alignment of surrounding collagen fibrils. In other words, the extent of fibril deformation and realignment (remodeling) by cells is decreased and more uniformly distributed around the cell in the presence of increased concentrations of hyaluronic acid.

Results of these studies show that while the addition of hyaluronic acid does not dramatically affect the 3D fibril microstructure of the resultant engineered matrices, it does affect the mechanical properties, likely by changing the properties of the viscous fluid component. Furthermore, systematic variation of the viscous fluid component as a specific design criteria for 3D engineered matrices does affect the mechanisms by which resident cells mechanically manipulate (contract) or remodel their ECM microenvironment.

1. A composition for supporting cell growth, said composition comprising
a three dimensional purified collagen matrix comprised of collagen fibrils, wherein the collagen component of the matrix consists essentially of type I and type III collagen, and the fibril area fraction of the three dimensional matrix is about 7.7% to about 25%.

2. The composition of claim 1 wherein the type I collagen to type III collagen ratio is selected from a ratio ranging from about 6:1 to about 1:1.

3. The composition of claim 1 wherein the ratio of type I to type III collagen is selected from a ratio ranging from about 200:1 to about 6:1.

4. The composition of claim 1 further comprising a population of cells entrapped within the three dimensional matrix.

5. The composition of claim 4 wherein the three dimensional matrix further comprises exogenously added glucose and calcium chloride.

6. The composition of claim 1 wherein the three dimensional matrix has an elastic or linear modulus of about 0.5 kPa to about 40.0 kPa.

7. The composition of claim 1 wherein the three dimensional matrix has an elastic or linear modulus of about 0.5 kPa to about 24 kPa.

8. The composition of claim 1 wherein the three dimensional matrix has an elastic or linear modulus of about 25.6 kPa to about 40.2 kPa.

9. The composition of claim 1 wherein the three dimensional matrix is synthesized by polymerizing a hydrochloric solution of solubilized purified type I and type III collagen.

10. A composite tissue graft comprising
a first three dimensional matrix; and
a second three dimensional matrix, wherein the first and second three dimensional matrices are physically connected to one another and the composition of the first and second three dimensional matrices are not the same.

11. The composite tissue graft of claim 10 wherein the first and second three dimensional matrices differ based on a property selected from the group consisting of fibril density, tensile strength, collagen content, and the fluid composition of the matrix.

12. The composite tissue graft of claim 11 wherein the first and second three dimensional matrices differ from one another based on the relative tensile strength of the two matrices.

13. The composite tissue graft of claim 11 wherein one of said first or second three dimensional matrices further comprises a population of cells entrapped within the respective first or second three dimensional matrix.

14. The composite tissue graft of claim 13 wherein the three dimensional matrix comprising the population of cells further comprises exogenously added glucose and calcium chloride.

15. The composite tissue graft of claim 14 wherein the population of cells are stem cells.

16. The composite tissue graft of claim 11 wherein both the first and second three dimensional matrices further comprises cells entrapped within their respective matrix, the cells entrapped within the first three dimensional matrix being the same or different than those entrapped in the second three dimensional matrix.

17. The composite tissue graft of claim 16 wherein the first and second three dimensional matrices further comprises exogenously added glucose and calcium chloride.

18. The composite tissue graft of claim 10 wherein the composite comprises a sheet of a first three dimensional matrix layered onto a sheet of a second three dimensional matrix to form a laminate structure.

19. The composite tissue graft of claim 18 wherein the laminate structure comprises three layers with the sheet of the first three dimensional matrix being sandwiched between two sheets of the second three dimensional matrix.

20. The composite tissue graft of claim 19 wherein the first three dimensional matrix is further provided with a population of cells embedded within the three dimensional matrix.

21. The composite tissue graft of claim 10 wherein multiple pieces of a first three dimensional matrix are provided, each of said pieces being suspended within and surrounded by the second three dimensional matrix.

22. The composite tissue graft of claim 21 wherein said multiple pieces of the first three dimensional matrix further comprise a population of cells entrapped within the first three dimensional matrix.

23. The composite tissue graft of claim 22 wherein the population of cells are stem cells.

24. The composite tissue graft of claim 23 wherein the second three dimensional matrix further comprises a population of cells entrapped within the second three dimensional matrix.

25. The composite tissue graft of claim 23 wherein the composite tissue graft further comprises exogenously added glucose and calcium chloride.

26. The composite tissue graft of claim 10 wherein the first three dimensional matrix has a fibril area fraction of about 7% to about 18% and a tensile strength of 0.48 to about 24.0 kPa, and the second three dimensional matrix has a fibril area fraction of about 19% to about 26% and a tensile strength of about 25 to about 40 kPa.

27. The composite tissue graft of claim 25 wherein the first three dimensional matrix has a fibril area fraction of about 7% to about 18% and a tensile strength of 0.48 to about 24.0 kPa, and the second three dimensional matrix has a fibril area fraction of about 19% to about 26% and a tensile strength of about 25 to about 40 kPa.

28. The composite graft of claim 10 wherein both the first and second three dimensional matrices are three dimensional purified collagen matrices.

29. The composite graft of claim 10 wherein one of said first and second three dimensional matrices is a three dimensional purified collagen matrix and the other is a three dimensional extracellular matrix.

30. The composite graft of claim 10 wherein both the first and second three dimensional matrices are three dimensional extracellular matrices.

31. A method for enhancing the repair of tissues in a warm blooded vertebrate, said method comprising
providing a first solubilized collagen composition;
providing a second solubilized collagen composition;
polymerizing the first solubilized collagen composition to form a plurality of first three dimensional matrix pieces;
suspending the plurality of first three dimensional matrix pieces in the second solubilized collagen composition to form a first matrix suspension;
inducing the polymerization of the second solubilized collagen composition; and
allowing the polymerization of the second solubilized collagen composition to form a composite tissue graft construct comprising the plurality of first three dimensional matrix pieces interspersed within, and surrounded by a second three dimensional matrix, wherein the composite tissue graft construct is inserted into the warm blooded vertebrate, wherein the presence of the inserted matrix enhances tissue repair or tissue function.

32. The method of claim 31 wherein the inserting step comprises injecting the first matrix suspension into the warm blooded vertebrate and allowing the polymerization of the second solubilized collagen component to occur in vivo.

33. The method of claim 31 wherein the polymerization of the second solubilized collagen component occurs in vitro, and the composite tissue graft construct is implanted into the warm blooded vertebrate.

34. The method of claim 31 wherein cells are added to the first solubilized collagen composition prior to the step of polymerizing the first solubilized collagen composition.

35. The method of claim 34 wherein the cells are stem cells.

36. The method of claim 35 wherein the cells are added at a density of less than 5×10⁴ cells per milliliter.

37. A method for preparing a composite construct, said method comprising
providing a first solubilized collagen composition;
providing a second solubilized collagen composition;
polymerizing the first solubilized collagen composition to form a plurality of first three dimensional matrix pieces;
suspending the plurality of first three dimensional matrix pieces in the second solubilized collagen composition to form a first matrix suspension;
inducing the polymerization of the second solubilized collagen composition; and
allowing the polymerization of the second solubilized collagen composition to form a composite tissue graft construct comprising the plurality of first three dimensional matrix pieces interspersed within, and surrounded by a second three dimensional matrix.
